SEQUENCE LISTING (52 sequences)

SEQ ID NO: 1 - protein, 313 aa, Jilunia laotingensis
MKTIKLKYTDFWDHFDPKNNFITNVLSKKYHIEFSDTPDYLIFSIFGYEHLKFQNCIKILYSGENITPDFNLCDYAIGFDHITFGDRYMRLPLYVSYDIERLAKPKAIDPEKVLNRKFCSFVVSNGVSPERNRFFKLLSEYKRVDSGGRFMNNIGGPVPDKEAFIKDYKFNIAFENSIYDGYTTEKLLEPMLMDSLPIYWGNKLVGLDFNPQSFINATDFPSLEAVVEYVVKLDTNDEDYLSKLSIPWLNKNNYLNWESQFYDFFDNIFNKPKSEARYLTDYGRVRIYRQRLNLFSTIDVIKRKLDDLRNRFR

SEQ ID NO: 2 - protein, 321 aa, Bacteroides gallinaceum
MKSLKIKFVDFWPGFNPNDNFITNALKGYEIVITDTPDYLFFSIFGYSHLKYNCVKIMFVGENIVPDFNLCDYAMGFDFLNFGDRYMRLPLFLICDNFKELSVTKDFSPKRMLNRKFCSIVVSNAQVSNPIRERFFRLLSEYKQVDSGGRLWNNVGGPVADKQKFISGYKFNIAFENSAVLGYTTEKIMDAMTANTLPIYWGNPWVGRDFNKRSFVNVNSFASLEKAVEYIVDLDTNDERYLEMMQEPWVNDVSIFDWEDKLCAFLAHIVEKPFAEAQYLVDDGMQKLYKQNMKTLAFVNEKLKVPRLISAYKKLSNGKYK

SEQ ID NO: 3 - protein, 323 aa, unidentified
MNNKIKINFVDFWGDFNKCDNFITRALSKKYEVAISDNPDYLFFGTFGYRHLNYNCVKIMFIGENIAPDFNLCDYALGFDFIQFGDRYMRLPLYCTYESFASLAQTRLPSDEALIDRKFCSMVVSNNRHASPHRERFFKLLSEYKQVDSGGRLWNNVGGPVANKLEFVSQYKFNIAFENSAVHGYTTEKIMEPMAVNTLPLYWGNPLVCKDFNAASFVNVNDFASMEDAVQHIVDLDNDDSKYLQMIKAPKVLDENIINWEERLLDFLSNIIEKPIEEARYLAPFGVQKLYREELLAYDILSRRLKVKKIVAAYDKLRKKIRG

SEQ ID NO: 4 - protein, 320 aa, unidentified
MRDVTVAFVDFWPAFDDKNNFILDVLRQKYNVKTVAASDCPDILFYSYFGIENLRHTEPVRVYFTGENDVPDFNLCDYAISFHHLSFANRHLRLPLYVIYSCFNDLRNGRILQCEDAENRDFCSMVVSNVKNSDMARLDFFNLLSGYKQVASGGRFANNVGGCVTDKLSFISGYKFNIAFENSRVEGYVTEKIIEAFSSGTLPIYWGDPLVANELNDKAFIDVSKFDSLDAAVKFVAALDNNREQYLEILNQPPLLNNPFLNWEEMLLDFLTPIVNELKKYRVDTGAIGTAYRHKMSREQIYNIVTLKKPFELFKHLFHS

SEQ ID NO: 5 - protein, 325 aa, Bacteroides finegoldii
MKKKVAFVNFWETFKPKDSLFYKILCKHFDIELVDLAQAEYVFFSVWGDEHLYAPNNAVKIYFTGENRTPNFNECDYAMGFERLSFRDRYIRFPLYCIYPEYLLLEKKQIGIDKGTLNTKKGFCSFTVSNGTCQPRNEMFHLLNSYKRVDSGGRYLNNIGGRVEDKFAFDKEHKFSIVFENFSYNGYCSEKIVQAFAAQTIPIYWGDTLCEETFNTKAFINVHKYKTFNEVLERVKEIDNNDDLYLEMLSHPALISEQEGYEGKISQLEDFLLNIFSQPHETARRRTRMSYHEIAKRREMMEVYYRSKMSIISRLYHWVKGSRLW

SEQ ID NO: 6 - protein, 336 aa, Prevotella sp.
MKKIAFIGFWDNFNPETMPLTKYIREFTDVEITDVKHADYVFFSVQSEKHWFASDDSIKIFYTGENLTPDFNACDYAIGFDWMEYGDRYMRLPLYYLYKDICEQSERKHLLNPEAVAKEKTGFCSITVSNSRRNPIFQTLFEELSKYKKVDSGGRWNNNIGGPVADKQAFDASHKFSIVCENSAHPGYTTEKLVQAFAANCIPIYWGDPEVARVFNTRAFINVPDYSSVEEVADIVRRIDTDEQYYHAMLREPVFNDEEYYKARQIEKLKEFLSYIFSQPKEKACRRNREFYGLKYVKDHRTMARATSNIFFNDYRHKVAVSLYYKFRNLIKRDKP

SEQ ID NO: 7 - protein, 314 aa, Coprobacter secundus
MKTIKVKFVDFWKGFDPRNNFLMDILKQRYHIELSESPDYLIFSVFGFTNLNYERCVKIFYTGENLTPDFNICDYAIGFDYLSFGDRYMRLPLYAVYGIEKLASPKVIDKEKVLKRKFCSYVVSNNIGAPERNRFFHLLSEYKKVDSGGRWENNVGGPVPDKLDFIKDYKFNIAFENSMYDGYTTEKIMEPMLVNSLPVYWGNRLINKDFNPASFINVSDFPSQEAAVEHIVMLDNNDDMYLSILSKPWFNDENYLDWKVRFFHFFDNIFNRPIDECKYLTPYGFCRHYRNQLRSARLLKQRLRQLSNPLRWFR

SEQ ID NO: 8 - protein, 315 aa, Bacteroides nordii
MKTIKVKFVDFWENFDPQHNFIANIISKKYRIELSDTPDYLFFSVFGYENIDYHNCTKIFYSGENITPDFNICDYAIGFNFLSFGDRYIRIPFYTAYGVQQLAAPKVIVPEVVLNRKFCSFVVSNAKGAPERERFFQLLSEYKQVDSGGRYKNNVGGPVPDKTAFIKDYKFNIAFENSMCDGYTTEKIMEPMLVNSVPIYWGNKLIDRDFNPDSFINVSSYSSLEEAVEHIVRLDQNDDEYLSLLSAPWFNEENYLNWEEQLITFFDNIFEKPLSESRYIPTHGYIQTYQYRLHRMMRDKLFRKRINPLKWFSSK

SEQ ID NO: 9 - protein, 425 aa, synthetic construct
MFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYNELHYKAELVNDTTAPYKLKGNSLYALKKPSHHFKENHPNLCAVVNDESDLLKRGFASFVASNANAPMRNAFYDALNSIEPVTGGGSVRNTLGCKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 10 - protein, 331 aa, Bacteroides fragilis
MCDCLSIILLVKMKKIYLKFVDFWDGFDTISNFIVDALSIQYEVVLSNEPDYLFYSCFGTSHLEYDCIKIMFIGENIVPDFNVCDYAIGFNYIDFGDRYLRLPLYAIYDGFSNLQNKKIDVNKALDRKFCSIVVSNNKWADPIRETFFKLLSSYKKVDSGGRAWNNIGGPVDNKLDFISQYKFNIAFENSRVLGYTTEKIMEPMQVNSIPVYWGNPLVGKDFNVDSFVNAHDFDSLERLVEYIIELDSSKDKYLEMLEKPWLLDKTYLDWKQLLLNFINNIMMKSYKDAKYLVNYGHAGKYRNEQRFWGRCERKFKLQRIIEYYSQLFDRK

SEQ ID NO: 11 - protein, 425 aa, Helicobacter pylori
MFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYAHLHYKAELVNDTTAPYKLKGNSLYALKKPSHHFKENHPNLCAVVNDESDLLKRGFASFVASNANAPMRNAFYDALNSIEPVTGGGSVRNTLGCKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 12 - DNA, 942 bp, synthetic construct
  source 1..942: mol_type = other DNA
atgaaaaccatcaagctgaaatataccgatttctgggatcatttcgacccgaaaaacaactttattaccaatgtgctgagcaaaaagtaccacatcgaatttagcgatacaccggattatctgatctttagcatctttggctatgagcacctgaaattccagaactgcattaaaatcctgtatagcggtgaaaacatcacaccggactttaatctgtgtgattatgccattggctttgaccatattacctttggtgatcgttatatgcgtctgccgctgtatgttagctatgatattgaacgtctggcaaaaccgaaagcaattgatccggaaaaagtgctgaatcgcaaattttgcagctttgttgttagcaatggtgttagtccggaacgtaatcgtttctttaaactgctgagcgaatataaacgtgttgatagcggtggtcgctttatgaataacattggtggtccggttccggataaagaagcattcatcaaagactataaattcaacattgccttcgagaacagcatctatgatggttataccaccgaaaaactgctggaaccgatgctgatggatagcctgccgatttattggggtaataaactggttggtctggattttaacccgcagagctttattaacgcaaccgattttccgagcctggaagcagttgttgaatatgttgttaaactggataccaacgatgaggactatctgagcaaactgagcattccgtggctgaacaaaaataactatctgaattgggagagccagttctatgatttcttcgataacatctttaacaaaccgaaaagcgaagcacgttatctgaccgattatggtcgtgttcgtatttatcgtcagcgtctgaacctgtttagcaccattgatgtgattaaacgcaaactggatgatctgcgtaatcgctttcgctaa

SEQ ID NO: 13 - DNA, 966 bp, synthetic construct
  source 1..966: mol_type = other DNA
atgaaaagcctgaagatcaagttcgtggatttttggcctggttttaatccgaacgataactttattaccaatgcgctgaaaggctatgagattgtgattaccgatacaccggattacctgtttttcagcatttttggttatagccacctgaagtacaactgcgtgaaaattatgtttgtgggcgaaaatatcgtgccggattttaatctgtgtgattatgccatgggcttcgactttctgaattttggtgatcgttatatgcgtctgccgctgtttctgatttgcgataatttcaaagaactgagcgtgaccaaagattttagcccgaaacgtatgctgaatcgcaaattttgtagcattgttgttagcaatgcccaggttagcaatccgattcgtgaacgtttctttcgtctgctgagcgaatataaacaggttgatagcggtggtcgtctgtggaataatgttggtggtccggttgcagataaacagaaatttatctccggctataaattcaacattgcctttgaaaatagcgcagtgctgggttataccaccgaaaagattatggatgcaatgaccgcaaataccctgccgatttattggggtaatccgtgggttggtcgtgatttcaataaacgtagctttgtgaacgtgaatagctttgcaagcctggaaaaagccgttgaatatattgttgatctggataccaacgatgagcgctatctggaaatgatgcaagaaccgtgggttaatgatgtgtccatttttgactgggaagataaactgtgtgcatttctggcacatattgtcgaaaaaccgtttgcagaagcacagtatctggttgatgatggtatgcagaaactgtacaagcagaatatgaaaaccctggcctttgtgaatgagaaactgaaagttccgcgtctgattagcgcatataagaaactgagcaacggcaagtacaaataa

SEQ ID NO: 14 - DNA, 972 bp, synthetic construct
  source 1..972: mol_type = other DNA
atgaacaacaagatcaagatcaacttcgtggatttctggggtgatttcaacaaatgcgataactttattacccgtgcgctgagcaaaaagtatgaagttgcaattagcgataacccggattacctgtttttcggcacctttggttatcgtcatctgaattataactgcgtgaaaatcatgttcatcggcgaaaatattgcaccggattttaacctgtgtgattatgcactgggctttgatttcattcagtttggtgatcgttatatgcgtctgccgctgtattgtacctatgaaagctttgcaagcctggcacagacccgtctgccgagtgatgaagcactgattgatcgtaaattttgcagcatggtggtgagcaataatcgtcatgcaagtccgcatcgtgaacgtttctttaaactgctgagcgaatataaacaggttgatagcggtggtcgtctgtggaataatgttggtggtccggttgcaaataaactggaatttgttagccagtacaaattcaacattgcctttgaaaatagcgcagtgcatggttataccaccgagaagattatggaaccgatggcagttaataccctgccgttatattggggtaatccgctggtttgcaaagactttaatgcagccagctttgtgaacgtgaatgattttgcaagtatggaagatgccgttcagcatattgttgatctggataacgatgacagcaaatacctgcagatgattaaagcaccgaaagtgctggatgaaaacatcattaattgggaagaacgcctgctggattttctgagcaatattatcgaaaaaccgatcgaggaagcacgttatctggcaccgtttggtgttcagaaactgtatcgtgaagaactgctggcatatgatattctgagccgtcgtctgaaagtgaaaaagattgttgcagcctatgataaactgcgcaaaaagattcgtggctaa

SEQ ID NO: 15 - DNA, 963 bp, synthetic construct
  source 1..963: mol_type = other DNA
atgcgtgatgttaccgttgcctttgttgatttttggcctgcctttgatgacaaaaacaactttattctggatgtgctgcgccagaaatataacgttaaaaccgttgcagcaagcgattgtccggatattctgttttatagctatttcggcattgaaaacctgcgtcataccgaaccggtgcgtgtgtattttacaggtgaaaatgatgtgccggatttcaacctgtgtgattatgcaattagctttcaccatctgagctttgcaaatcgtcatctgcgtctgccgctgtatgtgatttatagctgttttaatgatctgcgcaatggtcgtattctgcagtgtgaagatgcagaaaatcgtgatttttgcagcatggttgtgagcaatgtgaaaaatagcgatatggcacgtctggatttctttaatctgctgagcggttataaacaggttgcaagcggtggtcgttttgcaaataatgttggtggttgtgttaccgacaaactgagctttattagcggctacaaattcaacattgcctttgaaaattcacgcgtggaaggttatgtgaccgagaaaattatcgaagcatttagcagcggcaccctgccgatttattggggtgatccgctggttgcaaatgaactgaatgataaagccttcatcgacgtgagcaaatttgatagcctggatgcagcagttaaatttgttgcagcactggataataaccgcgaacagtatctggaaattctgaatcagcctccgctgctgaataatccgtttctgaattgggaagaaatgctgctggattttctgaccccgattgttaacgaactgaaaaagtatcgtgttgataccggtgcaattggtacagcatatcgtcataaaatgagccgtgagcagatttataacattgtgaccctgaaaaagccgttcgaactgttcaaacacctgtttcacagctaa

SEQ ID NO: 16 - DNA, 978 bp, synthetic construct
  source 1..978: mol_type = other DNA
atgaaaaagaaagtggccttcgtgaacttttgggaaacctttaaaccgaaagatagcctgttctacaaaatcctgtgcaaacactttgatatcgaactggttgatctggcacaggcagaatatgttttctttagcgtttggggtgatgaacatctgtatgcaccgaataatgccgtgaaaatctatttcacaggtgaaaatcgtaccccgaactttaacgaatgtgattatgcaatgggttttgaacgtctgagctttcgtgatcgttatattcgttttccgctgtattgcatctatccggaatatctgctgctggaaaagaagcagattggtattgataaaggcaccctgaataccaaaaagggcttttgtagctttaccgttagcaatggcacctgtcagcctcgtaatgaaatgtttcatctgctgaatagctataaacgtgttgatagcggtggtcgctatctgaataacattggtggtcgtgttgaggacaaattcgcctttgataaagaacacaaatttagcatcgtgttcgaaaacttcagctataatggttactgcagcgagaaaattgttcaggcatttgcagcacagaccattccgatttattggggagataccctgtgtgaagaaaccttcaatacaaaagcgtttatcaacgtgcacaaatacaagacctttaacgaggttctggaacgcgtgaaagaaatcgataataacgatgatctgtatctggaaatgctgagccatccggcactgattagcgaacaagagggttatgaaggtaaaattagccagctggaagatttcctgctgaacatttttagccagccgcatgaaaccgcacgtcgtcgtacccgtatgagctatcatgaaattgcaaaacgtcgcgaaatgatggaagtgtattatcgtagcaaaatgagcattatcagccgtctgtatcattgggttaaaggtagtcgtctgtggtaa

SEQ ID NO: 17 - DNA, 1011 bp, synthetic construct
  source 1..1011: mol_type = other DNA
atgaagaagattgcctttatcggcttctgggataactttaatccggaaaccatgccgctgaccaaatatatccgtgaatttaccgatgtggaaatcaccgatgttaaacatgcagattatgtgtttttcagcgtgcagagcgaaaaacattggtttgcaagtgatgacagcatcaagatcttttacacaggcgaaaatctgacaccggattttaatgcatgtgattatgccattggcttcgactggatggaatatggtgatcgttatatgcgtctgccgctgtattatctgtataaagatatttgcgaacagagcgagcgtaaacatctgctgaatccggaagcagttgcaaaagagaaaaccggcttttgtagcattaccgttagcaatagccgtcgcaatccgatttttcagaccctgtttgaagaactgagcaaatacaaaaaggttgatagcggtggtcgctggaataataacattggtggtccggttgcagataaacaggcatttgatgcaagccacaaatttagcattgtgtgcgaaaatagcgcacatccgggttataccaccgaaaaactggttcaggcctttgcagcaaattgcattccgatttattggggtgatccggaagttgcacgtgtgtttaatacccgtgcctttattaacgtgccggattatagcagcgttgaagaagttgcggatattgttcgtcgtattgataccgatgaacagtattatcatgcaatgctgcgtgaaccggtgtttaatgatgaagaatattacaaagcccgtcagatcgagaaactgaaagaatttctgagctatatcttcagccagccgaaagaaaaagcatgtcgtcgtaatcgtgaattctatggcctgaaatatgtgaaagatcatcgtaccatggcacgtgcgaccagcaatattttctttaatgattatcgccacaaagtggccgttagcctgtattacaaatttcgcaatctgatcaaacgcgacaaaccgtaa

SEQ ID NO: 18 - DNA, 945 bp, synthetic construct
  source 1..945: mol_type = other DNA
atgaaaaccatcaaggtgaagttcgtggatttttggaaaggttttgatccgcgtaacaacttcctgatggatattctgaaacagcgctatcatattgaactgagcgaaagtccggattatctgatctttagcgtttttggctttaccaacctgaattatgaacgctgcgtgaaaatcttttacaccggtgagaatctgacaccggattttaacatttgcgattatgccatcggctttgattatctgtcatttggtgatcgttatatgcgtctgccgctgtatgcagtttatggtattgaaaaactggcaagcccgaaagtgatcgacaaagaaaaagttctgaaacgcaaattctgcagctatgtggtgagcaataacattggtgcaccggaacgtaatcgtttctttcatctgctgagcgagtataaaaaggttgatagcggtggtcgttgggaaaataatgttggtggtccggttccggataaactggattttatcaaagactacaaattcaacattgcctttgaaaacagcatgtatgatggttacaccaccgagaaaatcatggaaccgatgctggttaatagcctgccggtttattggggtaatcgtctgattaacaaagactttaatccggcaagctttatcaacgttagcgattttccgagccaagaagcagcagttgaacatattgttatgctggataacaacgacgatatgtatctgagcattctgagcaaaccgtggtttaacgatgaaaactatctggattggaaagtgcgctttttccacttcttcgataacatttttaaccgtccgatcgacgagtgtaaatatctgaccccgtatggtttttgtcgccattatcgtaatcagctgcgtagcgcacgtctgctgaaacaacgtctgcgtcagctgagcaatccgctgcgttggtttcgttaa

SEQ ID NO: 19 - DNA, 948 bp, synthetic construct
  source 1..948: mol_type = other DNA
atgaaaaccatcaaggtgaagttcgtggatttttgggaaaattttgatccgcagcataactttatcgccaacatcatcagcaaaaagtatcgcattgaactgagcgatacaccggattacctgtttttcagcgtttttggctatgagaacatcgattatcacaactgcaccaagatcttttatagtggcgaaaatatcacaccggacttcaacatttgtgattatgccatcggtttcaactttctgagctttggtgatcgctatattcgcattccgttttataccgcatatggtgttcagcagctggcagcaccgaaagttattgttccggaagttgttctgaatcgcaaattttgcagctttgttgtgagcaatgcaaaaggtgcaccggaacgtgaacgtttctttcagctgctgtcagaatataaacaggttgatagcggtggtcgctacaaaaataacgttggtggtccggttccggataaaaccgcatttatcaaagactataaattcaacattgcctttgaaaacagcatgtgcgacggttataccaccgagaaaatcatggaaccgatgctggttaatagcgttccgatttattggggcaacaaactgattgatcgtgattttaatccggacagctttatcaacgtttcaagctatagcagcctggaagaagcagttgaacatattgttcgtctggatcagaacgatgatgaatatctgagcctgctgagcgcaccgtggtttaatgaagaaaattatctgaactgggaagaacagctgatcacctttttcgataacatctttgaaaaaccgctgagcgaaagccgttatattccgacacatggttatattcagacctatcagtatcgtctgcatcgtatgatgcgtgataaactgtttcgcaaacgtattaatccgctgaaatggtttagcagcaaataa

SEQ ID NO: 20 - DNA, 1278 bp, synthetic construct
  source 1..1278: mol_type = other DNA
atgttccaaccgctgctggacgcgttcatcgaatctgcctctattgaaaaaatggcctcgaaatcgccgccgccgccgctgaaaatcgcagtggctaattggtggggtgatgaagaaatcaaagaatttaaaaaatctgtgctgtatttcattctgtctcagcgttacgcaatcaccctgcatcaaaacccgaatgaatttagtgacctggtcttctccaacccgctgggtgcagcacgtaaaattctgagctatcagaataccaaacgcgtgttttacacgggtgaaaacgaatctccgaactttaacctgtttgattatgccatcggctttgatgaactggacttcaatgatcgttatctgcgcatgccgctgtattacaatgagctgcactataaagctgaactggttaatgataccacggcgccgtataaactgaagggtaacagtctgtacgccctgaaaaaaccgtcccatcactttaaagaaaaccacccgaatctgtgcgcggtggttaacgacgaaagcgatctgctgaaacgtggctttgcatcattcgttgcttcgaacgcgaatgccccgatgcgcaacgcgttttatgatgccctgaacagcattgaaccggttaccggcggtggctcggtccgtaatacgctgggttgcaaagtgggcaacaaaagcgaatttctgtctcagtacaaattcaacctgtgtttcgaaaatagtcaaggttatggctacgttaccgaaaaaatcctggatgcgtatttctcccatacgattccgatctactggggtagcccgtctgtcgccaaagattttaacccgaaatctttcgtcaatgtgcacgacttcaacaacttcgacgaagcaatcgattacatcaaatacctgcatacccacccgaatgcttatctggatatgctgtacgaaaacccgctgaatacgctggacggcaaagcctatttttaccaggatctgagtttcaagaaaattctggatttctttaaaaccattctggaaaacgatacgatctaccataaattcagtaccagctttatgtgggaatacgacctgcacaaaccgctggttagcatcgatgacctgcgtgttaactatgatgacctgcgtgtcaattacgatcgcctgctgcagaacgcatcaccgctgctggaactgtcgcaaaataccacgtttaaaatctatcgcaaagcgtatcaaaaatcactgccgctgctgcgtgctgtccgtaaactggtcaaaaaactgggtctgtaa

SEQ ID NO: 21 - DNA, 996 bp, synthetic construct
  source 1..996: mol_type = other DNA
atgtgtgattgcctgagcattattctgctggtgaaaatgaaaaagatctatctgaagttcgtggatttctgggatggctttgataccattagcaactttattgttgatgccctgagcatccagtatgaagttgttctgagcaatgaaccggattacctgttttatagctgttttggcaccagccatctggaatatgattgcatcaaaatcatgttcatcggcgaaaatatcgtgccggattttaatgtttgcgattatgccatcggcttcaactatattgattttggtgatcgttatctgcgtctgccgctgtatgcaatttatgatggttttagcaatctgcagaacaaaaagatcgatgtgaataaagccctggatcgcaaattttgcagcattgttgtttccaacaacaaatgggcagatccgattcgtgaaacctttttcaaactgctgagcagctataagaaagttgatagcggtggtcgtgcatggaataacattggtggtccggttgataataaactggatttcatcagccagtacaaatttaacattgcctttgaaaatagccgtgtgctgggttataccaccgagaaaatcatggaaccgatgcaggttaatagcattccggtttattggggtaatccgctggttggcaaagatttcaatgttgatagctttgtgaacgcccacgattttgatagcctggaacgtctggttgaatatatcattgaactggatagcagcaaggacaagtatctggaaatgctggaaaaaccgtggctgctggataaaacatatctggattggaaacagctgctgctgaactttattaacaacattatgatgaaaagctataaagatgccaaatatctggtgaactatggccatgcaggtaaatatcgtaatgaacagcgtttttggggtcgttgtgaacgtaaattcaagctgcagcgtatcatcgagtattacagccagctgtttgatcgcaagtaa

SEQ ID NO: 22 - DNA, 1278 bp, synthetic construct
  source 1..1278: mol_type = other DNA
atgttccaaccgctgctggacgcgttcatcgaatctgcctctattgaaaaaatggcctcgaaatcgccgccgccgccgctgaaaatcgcagtggctaattggtggggtgatgaagaaatcaaagaatttaaaaaatctgtgctgtatttcattctgtctcagcgttacgcaatcaccctgcatcaaaacccgaatgaatttagtgacctggtcttctccaacccgctgggtgcagcacgtaaaattctgagctatcagaataccaaacgcgtgttttacacgggtgaaaacgaatctccgaactttaacctgtttgattatgccatcggctttgatgaactggacttcaatgatcgttatctgcgcatgccgctgtattacgcacatctgcactataaagctgaactggttaatgataccacggcgccgtataaactgaagggtaacagtctgtacgccctgaaaaaaccgtcccatcactttaaagaaaaccacccgaatctgtgcgcggtggttaacgacgaaagcgatctgctgaaacgtggctttgcatcattcgttgcttcgaacgcgaatgccccgatgcgcaacgcgttttatgatgccctgaacagcattgaaccggttaccggcggtggctcggtccgtaatacgctgggttgcaaagtgggcaacaaaagcgaatttctgtctcagtacaaattcaacctgtgtttcgaaaatagtcaaggttatggctacgttaccgaaaaaatcctggatgcgtatttctcccatacgattccgatctactggggtagcccgtctgtcgccaaagattttaacccgaaatctttcgtcaatgtgcacgacttcaacaacttcgacgaagcaatcgattacatcaaatacctgcatacccacccgaatgcttatctggatatgctgtacgaaaacccgctgaatacgctggacggcaaagcctatttttaccaggatctgagtttcaagaaaattctggatttctttaaaaccattctggaaaacgatacgatctaccataaattcagtaccagctttatgtgggaatacgacctgcacaaaccgctggttagcatcgatgacctgcgtgttaactatgatgacctgcgtgtcaattacgatcgcctgctgcagaacgcatcaccgctgctggaactgtcgcaaaataccacgtttaaaatctatcgcaaagcgtatcaaaaatcactgccgctgctgcgtgctgtccgtaaactggtcaaaaaactgggtctgtaa

SEQ ID NO: 23 - DNA, 6706 bp, synthetic construct
  source 1..6706: mol_type = other DNA
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaatgatgtttttacgtcaggaagactttgccacggtagtgcgctccactccgcttgtctctctcgactttattgtcgagaacagtcgcggcgagtttctgcttggcaaaagaaccaaccgcccggcgcagggttactggtttgtgccgggagggcgcgtgcagaaagacgaaacgctggaagccgcatttgagcggctgacgatggcggaactggggctgcgtttgccgataacagcaggccagttttacggtgtctggcagcacttttatgacgataacttctctggcacggatttcaccactcactatgtggtgctcggttttcgcttcagagtatcggaagaagagctgttactgccggatgagcagcatgacgattaccgctggctgacgtcggacgcgctgctcgccagtgataatgttcatgctaacagccgcgcctattttctcgctgagaagcgtaccggagtacccggattatgaaaatactggtctacggcattaactactcgccggagttaaccggcatcggcaaatacaccggcgagatggtggaatggctggcggcacaaggtcatgaggtgcgggtcattaccgcaccgccttactacccgcaatggcaggtgggcgagaactattccgcctggcgctacaaacgagaagagggggccgccacggtgtggcgctgcccgctgtatgtgccaaaacagccgagcaccctgaaacgcctgttgcatctgggcagttttgccgtcagcagtttctttccgctgatggcgcaacgtcgctggaagccggatcgcattattggcgtggtgccaacgctgttttgcgcgccgggaatgcgcctgctggcgaaactctctggtgcgcgtaccgtgctgcatattcaggattacgaagtggacgccatgctggggctgggccttgccggaaaaggcaaaggcggcaaagtggcacagctggcaacggcgttcgaacgtagcggactgcataacgtcgataacgtctccacgatttcgcgttcgatgatgaataaagccatcgaaaaaggcgtggcggcggaaaacgtcatcttcttccccaactggtcggaaattgcccgttttcagcatgttgcagatgccgatgttgatgcccttcgtaaccagcttgacctgccggataacaaaaaaatcattctttactccggcaatattggtgaaaagcaggggctggaaaacgttattgaagctgccgatcgtctgcgcgatgaaccgctgatttttgccattgtcgggcagggcggcggcaaagcgcggctggaaaaaatggcgcagcagcgtggactgcgcaacatgcaatttttcccgctgcaatcgtatgacgctttacccgcactgctgaagatgggcgattgccatctggtggtgcaaaaacgcggcgcggcagatgccgtattgccgtcgaaactgaccaatattctggcagtaggcggtaacgcggtgattactgctgaagcctacacagaactggggcagctttgcgaaacctttccgggcattgcggtttgcgttgaaccggaatcggtcgaggcgctggtggcggggatccgtcaggcgctcctgctgcccaaacacaacacggtggcacgtgaatatgccgaacgcacgctcgataaagagaacgtgttacgtcaatttataaatgatattcggggataattatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 24 - protein, 332 aa, Neisseria meningitidis
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 25 - protein, 273 aa, Helicobacter pylori
MRVFAISLNQKVCDTFGLVFRDTTTLLNSINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYIRLMHLLYDASVKSEPLSHKNHEIQERVGIIKAYSEGVGTQGYVITPKIAKVFLKCSRKWVVPVDTIMDATFIHGVKNLVLQPFVIADDEQISTIARKEEPYSPKIALMRELHFKYLKYWQFV

SEQ ID NO: 26 - protein, 417 aa, Escherichia coli [sequence omitted]

SEQ ID NO: 27 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 28 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 29 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 30 - DNA, 141 bp, synthetic construct
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 31 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 32 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 33 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 34 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 35 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 36 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 37 - DNA, 189 bp, synthetic construct
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 38 - DNA, 239 bp, synthetic construct
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 39 - DNA, 300 bp, Escherichia coli
  source 1..300: mol_type = genomic DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 40 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 41 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 42 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 43 - DNA, 350 bp, synthetic construct
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 44 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 45 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 46 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 47 - DNA, 189 bp, synthetic construct; identical to SEQ ID NO: 37
  source 1..189: mol_type = other DNA

SEQ ID NO: 48 - DNA, 107 bp, Escherichia coli
  source 1..107: mol_type = genomic DNA
tgtgagttagctcactcattaggcaccccaggctttacactttatgcttccggctcgtatgttgtgtggaattgtgagcggataacaatttcacacaggaaacagct

SEQ ID NO: 49 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 50 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 51 - protein, 476 aa, Helicobacter pylori
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYDDLRVNYDDLRVNYDDLRVNYDDLRVNYDDLRVNYDDLRVNYDDLRVNYDRLLQNASPLLELSQNTTFKIYRKAYQKSLPLLRTIRRWVKK

SEQ ID NO: 52 - DNA, 1431 bp, synthetic construct
  source 1..1431: mol_type = other DNA
atgtttcagccgctgctggatgcatttattgaaagcgcaagcattgagaaaatggtgagcaaaagccctccgcctccgctgaaaattgcagttgcaaattggtggggtgatgaagaaatcaaagagtttaaaaagagcgtcctgtactttattctgagccagcgttatgcaattaccctgcatcagaatccgaatgaaagcagcgatctggtttttagcaatccgctgggtgcagcacgtaaaattctgagctatcagaataccaaacgcgtgttttataccggtgaaaatgaaagcccgaacttcaacctgtttgattatgccattggtttcgatgagctggattttaatgatcgttatctgcgtatgccgctgtattatgcacatctgcattatgaagcagaactggttaatgataccaccgcaccgtataaactgaaagataatagcctgtatgcactgaaaaagccgagccatcattttaaagaaaatcatccgaatctgtgtgccgtggtgaatgatgaaagcgatctgctgaaacgtggttttgcaagctttgttgcaagcaatgcaaatgcaccgatgcgtaatgcattttatgatgccctgaatagcattgaaccggttaccggtggtggtagcgttcgtaataccctgggttataaagttggcaacaaaagcgaattcctgagccagtataaattcaatctgtgctttgaaaacagccaaggctatggttatgtgaccgagaaaattctggatgcctatttcagccataccattccgatttattggggtagcccgagcgttgcaaaagatttcaatccgaaaagctttgtgaacgtgcacgatttcaacaactttgatgaagccatcgactacatcaaatatctgcatacccatccgaatgcctatctggatatgctgtatgaaaatccgctgaatacactggatggtaaagcctatttctatcaggatctgagcttcaaaaagatcctggactttttcaaaaccatcctggaaaacgataccatctatcacaataacccgttcatcttttatcgcgatctgcacgaaccgctgattagtattgatgatctgcgtgtgaattatgatgacctgcgcgttaactacgatgatttacgcgttaattatgatgatcttcgggtcaactatgatgacttaagagtgaactacgacgatctgagagtcaattatgatgatttgagagttaattatgatgacttgcgtgtgaattatgatgatttgcgggtgaactacgacgacctgcgtgttaactatgatcgtctgctgcagaatgcaagccctctgctggaactgagccagaatacaacctttaaaatctatcgcaaggcctaccagaaaagcctgcctctgctgcgtaccattcgtcgttgggttaaaaagtaa

(19) United States
(12) Patent Application Publication
(10) Pub. No.: US 2026/0317732 A1
(43) Pub. Date: Oct. 15, 2026

(54) NEW FUCOSYLTRANSFERASES FOR IN VIVO SYNTHESIS OF COMPLEX FUCOSYLATED HUMAN MILK OLIGOSACCHARIDES MIXTURES COMPRISING LNDFH-III

(71) Applicant: DSM IP Assets B.V., Maastricht (NL)

(72) Inventor: Manos PAPADAKIS, Hørsholm (DK)

(21) Appl. No.: 19/142,101

(22) PCT Filed: Dec. 21, 2023
(86) PCT No.: PCT/EP2023/087290

§ 371 Date: Jun. 20, 2025

(30) Foreign Application Priority Data
Dec. 22, 2022 (DK) PA202201202

(51) Int. Cl. C12N 9/10; C12P 19/04; C12P 19/18

(52) U.S. Cl. CPC C12N 9/1051; C12P 19/04; C12P 19/18

(57) ABSTRACT

The present disclosure relates to the production of complex fucosylated Human Milk Oligosaccharides (HMOs) and in particular to the production of complex fucosylated HMOs with five or more monosaccharide units, such as LNFP-III, LNFP-VI and LNDFH-III as well as mixtures thereof. The present disclosure also relates to genetically engineered cells and α-1,3-fucosyltransferases suitable for use in said production, as well as to methods for producing said fucosylated HMOs.

### CROSS-REFERENCE TO RELATED APPLICATIONS

This application is a national stage entry pursuant to 35 U.S.C. § 371 of International Patent Application No. PCT/EP2023/087290, filed on Dec. 21, 2023, which claims priority to Denmark Patent Application No. PA202201202, filed on Dec. 22, 2022, the entire contents of all of which are hereby incorporated by reference in their entirety.

### SEQUENCE LISTING

The computer-readable Sequence Listing submitted on Aug. 5, 2026, and identified as follows: 76,477 bytes ST.26 XML document file named “Sequence Listing.xml,” created Aug. 4, 2026, is incorporated herein by reference in its entirety.

### FIELD

The present disclosure relates to the production of complex fucosylated Human Milk Oligosaccharides (HMOs) and in particular to the production of complex fucosylated HMOs with five or more monosaccharide units, such as LNFP-III, LNFP-VI and LNDFH-III as well as mixtures thereof. The present disclosure also relates to genetically engineered cells suitable for use in said production.

### BACKGROUND

The design and construction of bacterial cell factories to produce fucosylated Human Milk Oligosaccharides (HMOs), especially for producing more complex fucosylated Human Milk Oligosaccharides (HMOs), is of paramount importance to provide innovative and scalable solutions for the more complex products of tomorrow.

Production of complex fucosylated HMOs either needs a multiple assortment of enzymes, or multi-specific enzymes, such as bi-specific glycosyltransferases capable of producing complex di-fucosylated HMOs. Multi-specific enzymes are preferred due to the lower genetical burden of introducing them to the host cell.

Production of complex fucosylated HMOs has e.g., been described in WO2019/008133, wherein the α1,3-fucosyltransferase FucT109 is suggested to fucosylate both the glucose (Glc) and N-acetylglucosamine (GlcNAc) moiety of Lacto-N-neotetraose (LNnT), thus potentially generating a mixture containing LNnT, LNFP-III and LNFP-VI (LNnFP-V). There is however no disclosure of production of LNDFH-III.

Dumon et al., 2004 (Biotechnol. Prog. 2004, 20, 412-419) further describes the α1,3-fucosyltransferases, FutA and FutB, which are suggested to produce a mixture of LNnT, (LNFP-III, in the case of FutB), LNFP-VI and LNDFH-III.

In summary, production of fucosylated HMOs, especially of more complex fucosylated HMOs, may be challenging due to the lack of α-1,3-fucosyltransferases with the desired substrate specificity, as well as low production yields of the desired fucosylated HMOs as compared to other HMO products present after fermentation, such as HMO precursor products, which may require laborious separation procedures.

### SUMMARY

The need for bi-specific glycosyltransferases for the production of complex di-fucosylated HMOs with an LNnT backbone, and in particular for the production of LNDFH-III, is in the present invention solved by the identification of a selection of α-1,3-fucosyltransferases which exhibit low or no specificity for the galactose moiety in LNnT as a substrate for fucosylation reactions, but which are highly substrate specific for the N-acetylglucosamine (GlcNAc) and glucose (Glu) moieties in LNnT, thus producing the complex fucosylated HMO LNDFH-III, or mixtures of HMOs that comprise LNDFH-III, and which have a high total content of fucosylated HMOs. The α-1,3-fucosyltransferases presented herein are therefore useful in the production of LNDFH-III. Hence, provided herein are enzymes, mixtures, compositions, uses, genetically engineered cells and methods for the production of LNDFH-Ill or mixtures of HMOs that comprise LNDFH-III, and which have a high total content of fucosylated HMOs.

A first aspect, relates to a genetically engineered cell capable of producing LNDFH-III, comprising a recombinant nucleic acid sequence encoding an α-1,3-fucosyltransferase selected from the group consisting of,
a. Osc1 comprising or consisting of the amino acid sequence of SEQ ID NO: 1, or a functional homologue thereof with an amino acid sequence that is at least 80% identical to SEQ ID NO: 1,
b. Bgall1 comprising or consisting of the amino acid sequence of SEQ ID NO: 2, or a functional homologue thereof with an amino acid sequence that is at least 80% identical to SEQ ID NO: 2,
c. Bbac1 comprising or consisting of the amino acid sequence of SEQ ID NO: 3, or a functional homologue thereof with an amino acid sequence that is at least 80% identical to SEQ ID NO: 3,
d. Murba1 comprising or consisting of the amino acid sequence of SEQ ID NO: 4, or a functional homologue thereof with an amino acid sequence that is at least 80% identical to SEQ ID NO: 4,
e. Bacfin1 comprising or consisting of the amino acid sequence of SEQ ID NO: 5, or a functional homologue thereof with an amino acid sequence that is at least 80% identical to SEQ ID NO: 5,
f. Prev1 comprising or consisting of the amino acid sequence of SEQ ID NO: 6, or a functional homologue thereof with an amino acid sequence that is at least 80% identical to SEQ ID NO: 6,
g. Csec1 comprising or consisting of the amino acid sequence of SEQ ID NO: 7, or a functional homologue thereof with an amino acid sequence that is at least 80% identical to SEQ ID NO: 7,
h. CafC comprising or consisting of the amino acid sequence of SEQ ID NO: 8, or a functional homologue thereof with an amino acid sequence that is at least 80% identical to SEQ ID NO: 8 and
i. FutA variants comprising substitutions at positions 128, 129 of SEQ ID NO: 11,
   wherein the variant has at least 80% identity, but less than 100% to SEQ ID NO: 11, and wherein the cell further comprises one or more recombinant nucleic acid sequence(s) encoding a β-1,4-galactosyltransferase and optionally a β-1,3-N-acetylglucosaminyltransferase.

A second aspect, relates to a method for producing one or more fucosylated HMOs, wherein one of the HMOs is LNDFH-III, said method comprising providing and culturing a genetically engineered cell capable of producing LNDFH-III, comprising a recombinant nucleic acid sequence encoding an α-1,3-fucosyltransferase selected from the group consisting of a) Osc1 comprising or consisting of the amino acid sequence of SEQ ID NO: 1, or a functional homologue thereof with an amino acid sequence that is at least 80% identical to SEQ ID NO: 1, b) Bgall1 comprising or consisting of the amino acid sequence of SEQ ID NO: 2, or a functional homologue thereof with an amino acid sequence that is at least 80% identical to SEQ ID NO: 2, c) Bbac1 comprising or consisting of the amino acid sequence of SEQ ID NO: 3, or a functional homologue thereof with an amino acid sequence that is at least 80% identical to SEQ ID NO: 3, d) Murba1 comprising or consisting of the amino acid sequence of SEQ ID NO: 4, or a functional homologue thereof with an amino acid sequence that is at least 80% identical to SEQ ID NO: 4, e) Bacfin1 comprising or consisting of the amino acid sequence of SEQ ID NO: 5, or a functional homologue thereof with an amino acid sequence that is at least 80% identical to SEQ ID NO: 5, f) Prev1 comprising or consisting of the amino acid sequence of SEQ ID NO: 6, or a functional homologue thereof with an amino acid sequence that is at least 80% identical to SEQ ID NO: 6, g) Csec1 comprising or consisting of the amino acid sequence of SEQ ID NO: 7, or a functional homologue thereof with an amino acid sequence that is at least 80% identical to SEQ ID NO: 7, h) CafC comprising or consisting of the amino acid sequence of SEQ ID NO: 8, or a functional homologue thereof with an amino acid sequence that is at least 80% identical to SEQ ID NO: 8, i) FucT109 comprising or consisting of the amino acid sequence of SEQ ID NO: 10, or a functional homologue thereof with an amino acid sequence that is at least 80% identical to SEQ ID NO: 10, and j) FutA variants comprising substitutions at a position corresponding to position 128 and 129 of SEQ ID NO: 11, wherein the variants have at least 80% sequence identity to SEQ ID NO: 11.

Preferably, the genetically engineered used in the method for producing LNDFH-III further comprises one or more recombinant nucleic acid sequence(s) encoding a β-1,4-galactosyltransferase and optionally a β-1,3-N-acetylglucosaminyltransferase.

A third aspect, relates to use of an α-1,3-fucosyltransferase in the production of one or more fucosylated HMOs, wherein the enzyme is selected from the group consisting of Osc1, Bgall1, Bbac1, Murba1, Bacfin1, Prev1, Csec1, and CafC comprising or consisting of an amino acid sequence according to SEQ ID NO: 1, 2, 3, 4, 5, 6, 7 or 8, or a functional homologue thereof with an amino acid sequence that is at least 80% identical to SEQ ID NO: 1, 2, 3, 4, 5, 6, 7 or 8, or wherein the enzyme is a FutA variant comprising substitutions at positions 128, 129 of SEQ ID NO: 11, wherein the variant has at least 80% identity, but less than 100% to SEQ ID NO: 11.

A fourth aspect relates to a mixture of HMOs produced with a method according to the present invention, consisting essentially of
a. LNDFH-III and 3FL, or
b. LNDFH-III, LNFP-III and 3FL, or
c. LNDFH-III, LNFP-VI and 3FL, or
d. LNDFH-III, LNFP-III, LNFP-VI and LNnT, or
e. LNDFH-III, LNFP-III, 3FL and LNnT, or
f. LNDFH-III, LNFP-III, LNFP-VI, 3FL and LNnT.

A fifth aspect relates to a composition of HMOs consisting essentially of 20-70 molar % of LNDFH-III, 0-35 molar % LNFP-III, 0-35 molar % LNDFP-VI, 0-65 molar % 3FL, 0-40% LNnT, and below 1% pLNnH, in total adding up to 100% molar content.

The invention further relates to compositions comprising the mixtures of a)-f), including synbiotic mixtures and use of said compositions in an infant formula, a dietary supplement, or medical nutrition.

### BRIEF DESCRIPTION OF FIGURES

FIG. 1. Overview of the synthesis of complex fucosylated HMO with an LNnT-backbone.

FIG. 2: Shows the experimental setup of the regeneration and viability assessment of lyophilized probiotics under pH 3.0 acidic conditions.

FIG. 3: Shows the regeneration and viability of lyophilized Lactobacillus rhamnosus (DSM 33156), incubated for 3 h at pH 3.0 and plated in up to 4 dilutions 1:1000 (E-3), 1:10,000 (E-4), 1:100,000 (E-5) and 1:1,000,000 (E-6). A) is the control without HMOs; B) is Lactobacillus rhamnosus (DSM 33156) in combination with an HMO mixture containing 50% LNDFH-III and 50% 3FL (mix 1); C) is Lactobacillus rhamnosus (DSM 33156) in combination with an HMO mixture containing 70% LNDFH-III and 20% 3FL and 10% LNFP-III (mix2); D) is Lactobacillus rhamnosus (DSM 33156) in combination with an HMO mixture containing 60% LNDFH-III, 10% 3FL, 20% LNFP-III and 10% LNnT (mix3); and E) is Lactobacillus rhamnosus (DSM 33156) in combination with an HMO mixture containing 45% LNDFH-III, 10% 3FL, 30% LNFP-III and 15% LNnT (mix 4).

### DETAILED DESCRIPTION

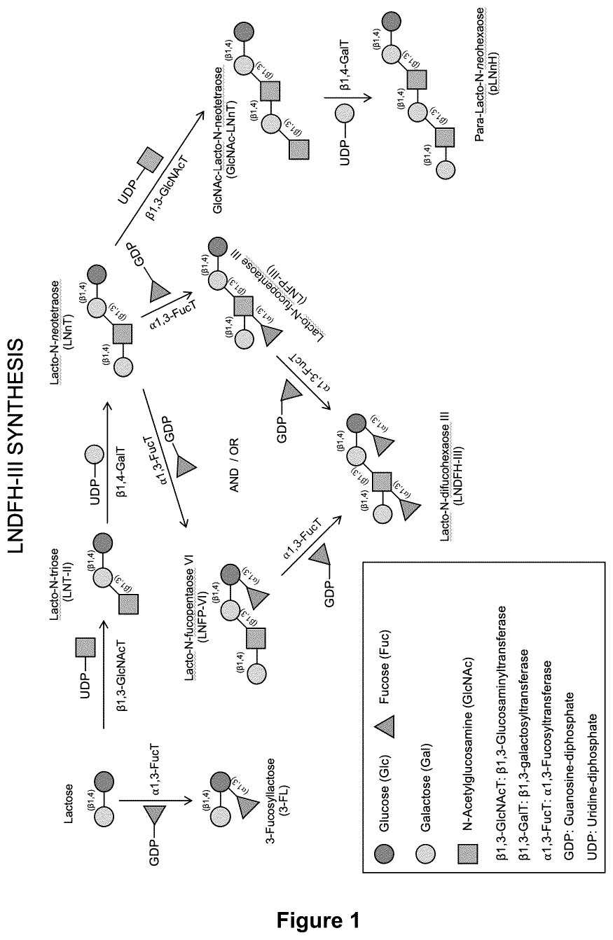

The present invention approaches the biotechnological challenges of in vivo HMO production of, in particular, complex fucosylated HMOs with an LNnT-backbone, which comprise at least five monosaccharide units, of which at least one monosaccharide unit is a fucosyl unit, in particular the di-fucosylated complex HMO with at least six monosaccharide units. Examples of such complex fucosylated HMOs are LNFP-III, LNFP-VI and LNDFH-III. The present invention offers specific strain engineering solutions for producing specific complex fucosylated HMOs, in particular, LNFP-III, LNFP-VI and/or LNDFH-III, by exploiting the dual substrate specificity of the α-1,3-fucosyltransferases disclosed herein towards the N-acetylglucosamine (GlcNAc) and Glucose (Glc) moieties in LNnT (see FIG. 1), making the enzyme capable of adding two fucosyl units in an α-1,3 binding to the LNnT backbone to form LNDFH-III.

The complex fucosylated HMOs with the LNnT backbone can be produced from lactose as the initial substrate, but in so far, the cell is capable of internalizing for example LNT-II or LNnT these may also serve as initial substrates.

Production of LNFP-III, LNFP-VI and/or LNDFH-III

The advantage of using the α-1,3-fucosyltransferases of the present disclosure is their ability to specifically recognize and fucosylate both the GlcNAc and Glucose moieties in LNT, to generate LNFP-III and/or LNFP-VI, which is in turn further fucosylated at the GlcNAc or Glucose moiety to produce the di-fucosylated HMOs, LNDFH-III, (see FIG. 1). In essence, the present disclosure describes several newly identified enzymes with dual α-1,3-fucosyltransferase activity that are capable of producing the complex fucosylated HMOs LNFP-III, LNFP-VI and/or LNDFH-II.

Furthermore, the α-1,3-fucosyltransferases described herein once introduced into a suitable cell produce different ratios of the different complex fucosylated HMO. Thus, if LNnT is available in sufficient amounts inside the genetically engineered cell, different levels of LNFP-III, LNFP-VI and/or LNDFH-III are produced.

The traits of the α-1,3-fucosyltransferases described herein are therefore well-suited for high-level industrial production of LNDFH-III and mixtures thereof comprising additional fucosylated species, without production of high levels of by-product HMOs, such as LNT-II and LNnT.

Accordingly, an α-1,3-fucosyltransferase described herein is well-suited for producing LNDFH-III and mixtures comprising LNDFH-III, LNFP-III, LNFP-VI, LNnT and/or 3FL. Some of the α-1,3-fucosyltransferase described herein, Bgall1, Bacfin1, Murba1 and FutA_mut2, primarily produce mixtures comprising LNDFH-III, LNFP-III, LNnT and 3FL, where the LNnT levels preferably are below 20%, such as below 15%, such as below 10% of the molar content of the total HMOs produced.

In particular, the α-1,3-fucosyltransferases which produce less than 10% of LNFP-III, such as less than 5% and less than 10% of LNFP-V, such as less than 5% of the molar content of the total HMOs produced are suitable for production of LNDFH-III as this simplifies the purification of LNDFH-III from the culture broth since it is easier to separate LNFDH-III and 3FL based on their size difference, than separation of LNDFH-III from LNFP-III and/or LNFP-VI. Examples of such α-1,3-fucosyltransferases described herein are Bgall1, Osc1 and Bbac1.

Some of the α-1,3-fucosyltransferase described herein, Bbac1, Prev1, FucT109 and Csec1, produce a mixture of HMOs which contain all three complex HMOs, LNFP-III, LNFP-VI and LNDFH-III, as well as LNnT and potentially also 3FL. In particular FucT109 appears to be able to produce a mixture of LNFP-III, LNFP-VI, LNDFH-III and LNnT, where all four HMOs are in the range from 15% to 30% of the molar content of the total HMOs produced.

The genetically engineered cells of the present disclosure, which express any one or more of the α-1,3-fucosyltransferases disclosed herein, with high substrate specificity for the GlcNAc and Glucose moieties in LNnT, for the first time enable the production of high titers of LNDFH-III which exceed 25%, such as exceeds 28%, such as exceeds 35%, such as such as exceeds 40%, such as exceeds 45%. such as exceeds 50% of the total amount of HMO produced. From the mixtures of HMOs produced by the genetically engineered cells LNDFH-Ill as well as LNFP-III and/or LNFP-VI can potentially be purified.

Thereby, the present disclosure enables a more efficient biotechnological production of more complex fucosylated HMOs, selected from the group consisting of LNFP-III, LNFP-VI and LNDFH-III, either in purified form, or alternatively as mixtures with the fucosylated HMOs being the most predominant, e.g., exceeding 65% of the total HMOs produced, preferably the fucosylated HMOs constitute at least 85%, such as at least 90%, such as at least 95% of the total amount of HMO produced.

In the following sections, individual elements of the invention, and in particular of the genetically engineered cell are described. It is understood that these elements can be combined across the individual sections.

### Oligosaccharides

In the present context, the term “oligosaccharide” means a sugar polymer containing at least three monosaccharide units, i.e., a tri-, tetra-, penta-, hexa- or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. N-acetylneuraminic acid). Preferably, the oligosaccharide is an HMO.

### Human Milk Oligosaccharide (HMO)

Preferred oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an α-L-fucopyranosyl and/or an α-N-acetyl-neuraminyl (fucosyl) moiety. HMO structures are e.g., disclosed by Xi Chen in Chapter 4 of Advances in Carbohydrate Chemistry and Biochemistry 2015 vol 72.

The present disclosure focuses on fucosylated HMO's. Examples of fucosylated HMOs include, 2′-fucosyllactose (2′FL), lacto-N-fucopentaose I (LNFP-I), lacto-N-difucohexaose I (LNDFH-I), 3-fucosyllactose (3FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-fucopentaose VI (LNFP-VI), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II), fucosyl-lacto-N-neohexaose (FLNnH), 3-fucosyl-3′-fucosyllactose (FSL), fucosyl-LST-a (FLST-a), fucosyl-LST b (FLST b), fucosyl-LST-c (FLST-c), fucosyl-LST d (FLST-d) and fucosyl-lacto-N-hexaose (SLNH).

In the context described herein, complex fucosylated HMOs are fucosylated HMOs that comprises at least 5 monosaccharide units of which at least one monosaccharide unit is a fucosyl unit, non-limiting examples of complex fucosylated HMOs are the fucosylated HMOs consisting of 5 monosaccharide units e.g., LNFP-I, LNFP-II, LNFP-III, LNFP-V and LNFP-VI and complex fucosylated HMO with 6 monosaccharide units such as but not limited to the di-fucosylated HMOs LNDFH-I, LNDFH-II and LNDFH-III or the sialyl-fucosyl HMOs FLST-a, FLST-b, FLST-c and FLST-d. Preferably, a complex fucosylated HMO is one that requires at least three different glycosyltransferase activities to be produced from lactose as the initial substrate, e.g., the formation of LNFP-III or LNFP-VI requires an α-1,3-fucosyltransferase, a β-1,3-N-acetyl-glucosaminyl-transferase and a β-1,4-galactosyltransferase (see FIG. 1), and the formation of LNDFH-II requires at least one α-1,3-fucosyltransferase, a β-1,3-N-acetyl-glucosaminyl-transferase and a β-1,4-galactosyltransferase, wherein the at least one α-1,3-fucosyltransferase, may be a single α-1,3-fucosyltransferase with dual activity on both the Glc and GlcNAc moieties of LNnT or it may be two different α-1,3-fucosyltransferases, one α-1,3-fucosyltransferase specific for the Glc moiety in LNFP-III and one α-1,3-fucosyltransferase specific for the GlcNAc moiety in LNFP-VI. Enzymes described herein preferably has dual activity on both the Glc and GlcNAc moieties of LNnT, thus being capable of producing LNDFH-III from LNnT (see FIG. 1).

In particular, the fucosylated HMO(s) produced is/are selected from complex fucosylated HMOs comprising at least five monosaccharide units of which at least one monosaccharide unit is a fucosyl unit. Moreover, in embodiments, the fucosylated HMOs is/are selected from complex fucosylated HMOs with an LNnT backbone structure, preferably, selected from the group consisting of LNFP-III, LNFP-VI and LNDFH-III. Examples of fucosylated HMOs with an LNnT backbone structure are lacto-N-fucopentaose III (LNFP-II), lacto-N-fucopentaose VI (LNFP-V), Lacto-N-difucohexaose III (LNDFH-III), sialyl-lacto-N-fucopentaose III (S-LNFP-III), Mono-Fucosyl-lacto-N-hexaose III (F-LNH-III), Difucosyl-Lacto-N-hexaose III (DF-LNH-III), Trifucosyl-lacto-N-neohexaose (TF-LNnH), Fucosyl-sialyl-lacto-N-neohexaose I (FS-LNnH) and Disialyl-fucosyl-lacto-N-neohexaose II (DS-F-LNnH-II).

In embodiments of the present disclosure, the α-1,3-fucosyltransferases described herein predominantly fucosylates both the N-acetylglucoseamine (GlcNAc) and Glucose (Glc) moieties of LNnT while also being capable of fucosylating the Glucose (Glc) moiety of lactose. In further embodiments, the α-1,3-fucosyltransferase described herein only fucosylates the N-acetylglucoseamine (GlcNAc) and Glucose (Glc) moiety of LNnT. In that regard, the one or more fucosylated HMOs is/preferably LNDFH-III and 3FL, or LNDFH-III, LNFP-III and 3FL, or LNDFH-III, LNFP-VI and 3FL, or LNDFH-III, LNFP-III, LNFP-VI and 3FL.

In human milk, about 60% of the content of HMOs are fucosylated HMOs, thus production of mixtures comprising a high content of fucosylated HMOs is highly desirable for the production of more natural mixtures of HMOs. In embodiments of the present disclosure at least 60 molar %, such as at least 70 molar %, 80 molar %, 85 molar %, 87 molar %, 89 molar %, 90 molar %, 91 molar %, 92 molar %, 93 molar %, 94 molar %, 95 molar %, 96 molar %, 97 molar %, 98 molar %, 99 molar or at least 99.5 molar %, of the produced HMOs are fucosylated HMOs.

### An Acceptor Oligosaccharide

A genetically engineered cell according to the present invention comprises a recombinant nucleic acid sequence encoding an α-1,3-fucosyltransferase capable of transferring fucose from an activated sugar to the GlcNAc and/or Glc moiety of an acceptor oligosaccharide, in an α-1,3 linkage on GlcNAc moiety or α-1,3 linkage on the Glc moiety.

As described herein, an acceptor oligosaccharide is an oligosaccharide that can act as a substrate for a glycosyltransferase capable of transferring a glycosyl moiety from a glycosyl donor to the acceptor oligosaccharide. The glycosyl donor is preferably a nucleotide-activated sugar as described in the section on “Glycosyl-donor-nucleotide-activated sugar pathways”. Preferably, the acceptor oligosaccharide is a precursor for making a more complex HMO and can also be termed the precursor molecule.

The acceptor oligosaccharide can be either an intermediate product of the present fermentation process, an end-product of a separate fermentation process employing a separate genetically engineered cell, or an enzymatically or chemically produced molecule.

In the present context, said acceptor oligosaccharide for the α-1,3-fucosyltransferase is preferably lacto-N-neotetraose (LNnT), which is produced from the precursor molecule lacto-N-triose II (LNT-II) (e.g., acceptor for the β-1,3-galactosyltransferase), which is produced from the initial precursor molecule lactose (e.g., acceptor for the β-1,3-N-acetyl-glucosaminyl-transferase) (see FIG. 1). In addition, the acceptor oligosaccharide for the α-1,3-fucosyltransferase may also be lacto-N-fucopentaose III (LNFP-III) or lacto-N-fucopentaose VI (LNFP-VI), which are produced from the precursor molecule LNnT (e.g., acceptor for the α-1,3-fucosyltransferase). The initial precursor molecule is preferably fed to the genetically engineered cell, which is capable of producing e.g., LNT-II, LNT, LNFP-III, LNFP-VI and/or LNDFH-III from the precursor. Most often the initial precursor is lactose and the genetically engineered cell is capable of producing the intermediate precursors (acceptor oligosaccharides, e.g. LNT-II and LNnT) inside the cell. The initial precursor may however also be LNT-II or LNT if the cell is capable of importing at least one of these compounds.

### Glycosyltransferases

The genetically engineered cell according to the present invention comprises at least one recombinant nucleic acid sequence encoding at least one glycosyltransferase, e.g., a fucosyltransferase, capable of transferring a fucosyl residue from a fucosyl donor to an acceptor oligosaccharide to synthesize one or more fucosylated human milk oligosaccharide product, i.e., a fucosyltransferase.

The genetically engineered cell according to the present invention may comprise one or more further recombinant nucleic acids encoding one or more recombinant and/or heterologous glycosyltransferases capable of transferring a glycosyl residue from a glycosyl donor to an acceptor oligosaccharide. Preferably, the additional glycosyltransferase(s) enables the genetically engineered cell to synthesize LNnT from a precursor molecule, such as lactose or LNT-II. In embodiments, the genetically engineered cell described herein, comprises one or more further recombinant nucleic acid encoding one or more recombinant and/or heterologous glycosyltransferase.

The additional glycosyltransferase is preferably selected from the group consisting of, galactosyltransferases, glucosaminyltransferases, fucosyltransferases N-acetylglucosaminyl transferases and sialyltransferases.

The fucosyltransferase in the genetically engineered cell described herein is an α-1,3-fucosyltransferase. Preferably, the α-1,3-fucosyltransferase is capable of transferring a fucose unit onto the GlcNAc and/or Glc moiety of LNnT, LNFP-III and/or LNFP-VI.

In the present disclosure, the functional enzyme (α-1,3-fucosyltransferase) capable of transferring a fucosyl moiety from a fucosyl donor to an acceptor oligosaccharide is selected from the group consisting of a) Osc1 comprising or consisting of the amino acid sequence of SEQ ID NO: 1, or a functional homologue thereof with an amino acid sequence that is at least 80% identical to SEQ ID NO: 1, b) Bgall1 comprising or consisting of the amino acid sequence of SEQ ID NO: 2, or a functional homologue thereof with an amino acid sequence that is at least 80% identical to SEQ ID NO: 2, c) Bbac1 comprising or consisting of the amino acid sequence of SEQ ID NO: 3, or a functional homologue thereof with an amino acid sequence that is at least 80% identical to SEQ ID NO: 3, d) Murba1 comprising or consisting of the amino acid sequence of SEQ ID NO: 4, or a functional homologue thereof with an amino acid sequence that is at least 80% identical to SEQ ID NO: 4, e) Bacfin1 comprising or consisting of the amino acid sequence of SEQ ID NO: 5, or a functional homologue thereof with an amino acid sequence that is at least 80% identical to SEQ ID NO: 5, f) Prev1 comprising or consisting of the amino acid sequence of SEQ ID NO: 6, or a functional homologue thereof with an amino acid sequence that is at least 80% identical to SEQ ID NO: 6, g) Csec1 comprising or consisting of the amino acid sequence of SEQ ID NO: 7, or a functional homologue thereof with an amino acid sequence that is at least 80% identical to SEQ ID NO: 7, h) CafC comprising or consisting of the amino acid sequence of SEQ ID NO: 8, or a functional homologue thereof with an amino acid sequence that is at least 80% identical to SEQ ID NO: 8, i) FucT109 comprising or consisting of the amino acid sequence of SEQ ID NO: 10, or a functional homologue thereof with an amino acid sequence that is at least 80% identical to SEQ ID NO: 10 and j) FutA variants comprising substitutions at a position corresponding to position 128 and 129 of SEQ ID NO: 11, wherein the variants have at least 80% sequence identity to SEQ ID NO: 11 and k) FutA_mut2 comprising or consisting of the amino acid sequence of SEQ ID NO: 9, or a functional homologue thereof with an amino acid sequence that is at least 80% identical to SEQ ID NO: 9. These enzymes can e.g., be used to produce LNFP-III, LNFP-VI and/or LNDFH-III.

Without being bound by theory, an α-1,3-fucosyltransferase that predominantly produces mixtures of LNDFH-III and LNFP-III and/or LNFP-VI, is advantageous as such an α-1,3-fucosyltransferase would in theory produce mixtures of HMO comprising one or more complex fucosylated HMOs, such as LNFP-III and LNDFH-III, or LNDFH-III and LNFP-VI, or LNDFH-III, LNFP-III and LNFP-VI, when the initial substrate is lactose and wherein the availability of LNnT is not limited. A high amount of complex fucosylated HMOs in the mixture of HMOs produced would result in an easier purification of the produced complex fucosylated HMOs, as the purification of LNDFH-III, LNFP-III and/or LNFP-VI from a mixture of HMOs predominantly comprising the intended product would be simpler, as it is easier to separate the complex fucosylated HMOs from smaller HMOs than separating different complex fucosylated HMOs of the same or similar size from each other, e.g., LNDFH-III from LNFP-III, or LNDFH-III from LNFP-VI. Hence in the production of LNDFH-III a lower initial amount of both LNFP-III or LNFP-VI is considered beneficial for the purification of LNDFH-III, while a lower initial amount of LNFP-VI is beneficial in the production of mixtures of LNDFH-Ill and LNFP-III, and a lower initial amount of LNFP-III is beneficial in the production of mixtures of LNDFH-III and LNFP-VI. A high amount of LNDFH-III, LNFP-III and LNFP-VI when a mixture comprising same is intended, is beneficial as it also simplifies the further purification of the mixture. In example, the α-1,3-fucosyltransferase Osc-1 produces essentially LNDFH-III as the sole complex fucosylated HMO, whereas the α-1,3-fucosyltransferase Bacfin1, Bgall1, Murba1 and FutA_mut2 produces essentially LNDFH-III and LNFP-III as the sole complex fucosylated HMOs, while the α-1,3-fucosyltransferases CafC, produces essentially LNDFH-III and LNFP-VI as the sole complex fucosylated HMO, and the α-1,3-fucosyltransferases FucT109, Prev1 and Csec1 produces essentially a mixture of LNDFH-III, LNFP-III and LNFP-VI.

In preferred embodiments, the use of an α-1,3-fucosyltransferase according to the present invention results in that at least 60 molar %, such as at least 70 molar %, 75 molar %, 80 molar %, 85 molar %, 90 molar %, 93 molar %, 95 molar %, 96 molar %, 97 molar % or such as at least 98 molar % of the molar content of the total HMOs produced by a cell according to the present invention is fucosylated HMOs.

In preferred embodiments, the use of an α-1,3-fucosyltransferase according to the present invention results in that at least 20 molar %, such as at least 23 molar %, such as at least 25 molar %, 27 molar %, 30 molar %, 33 molar %, 35 molar %, 38 molar %, 40 molar %, 42 molar %, 45 molar %, 50 molar %, 55 molar % or such as at least 60 molar % of the molar content of the total HMOs produced by a cell according to the present invention is LNDFH-III.

In further embodiments, the use of an α-1,3-fucosyltransferase according to the present invention results in that at least 50 molar %, such as at least 54 molar %, 60 molar %, 70 molar %, 75 molar %, 80 molar %, 85 molar %, 90 molar %, or such as at least 94 molar %, or such as between 50 molar % and 90 molar % or such as between 70 molar % and 94 molar % of the molar content of the total HMOs produced by a cell according to the present invention is a mixture of LNDFH-III and LNFP-III.

In further embodiments, the use of an α-1,3-fucosyltransferase according to the present invention results in that at least 60 molar %, such as at least 65 molar %, 70 molar %, 75 molar %, 80 molar %, 85 molar %, or such as at least 89 molar %, or such as between 60 molar % and 90 molar % of the molar content of the total HMOs produced by a cell according to the present invention is a mixture of LNDFH-III and LNFP-VI.

In further embodiments, the use of an α-1,3-fucosyltransferase according to the present invention results in that at least 55 molar %, such as at least 59 molar %, 65 molar %, 68 molar %, 70 molar %, 75 molar %, 70 molar %, or such as at least 85 molar %, or such as between 59 molar % and 86 molar % of the molar content of the total HMOs produced by a cell according to the present invention is a mixture of LNDFH-VI, LNFP-III and LNFP-VI.

In embodiments the α-1,3-fucosyltransferase is a FutA variant comprising two substitutions at a position corresponding to position 128 and 129 of SEQ ID NO: 11, wherein the variants have at least 80% sequence identity to SEQ ID NO: 11, such as at least 85% identity, such as at least 90% identity such as at least 95% identity such as 99.5% identity to SEQ ID NO: 11.

In embodiments the α-1,3-fucosyltransferase is a FutA variant which has at least 80% identity to SEQ ID NO: 11, wherein the amino acid corresponding to position 128 of SEQ ID NO: 11 is an asparagine or glutamine and/or the amino acid corresponding position 129 of SEQ ID NO: 11 is a glutamic acid or aspartic acid. Preferably, the FutA variant has at least 85% identity, such as at least 90% identity such as at least 95% identity such as 99.5% identity to SEQ ID NO: 11.

In one embodiment the FutA_mut2 variant comprises the following substitutions A128N or A128Q and H129E or H129D as compared to SEQ ID NO: 11, wherein the variant has at least 80% identity to SEQ ID NO: 11, such as at least 85% identity, such as at least 90% identity, such as at least 95% identity, or such as 99.5% identity to SEQ ID NO: 11.

In one embodiment the FutA_mut2 variant comprises the following substitutions A128N and H129E as compared to SEQ ID NO: 11, wherein the variant has at least 80% identity to SEQ ID NO: 11, such as at least 85% identity, such as at least 90% identity such as at least 95% identity such as 99.5% identity to SEQ ID NO: 11

In another embodiment the FutA_mut2 variant comprises the following the substitutions A128N, H129E, D148G and Y221C as compared to NCBI ref. No. WP_000487428.1, wherein the variant has at least 80% identity to WP_000487428.1, such as at least 85% identity, such as at least 90% identity such as at least 95% identity such as 99.1% identity to WP_000487428.1.

In embodiments the FutA variant is FutA_mut2 comprising or consisting of the amino acid sequence of SEQ ID NO: 9.

In embodiments, the expression of an α-1,3-fucosyltransferase described herein in a genetically engineered cell is further combined with expression of one or more further recombinant nucleic acids encoding one or more recombinant and/or heterologous glycosyltransferases.

In embodiments the cell further comprises one or more recombinant nucleic acid sequence encoding a β-1,4-galactosyltransferase.

In preferred embodiments, the expression of an α-1,3-fucosyltransferase of the invention in a genetically engineered cell is combined with expression of a β-1,4-galactosyltransferase such as galT from Helicobacter pylori. In a further embodiment, a third enzyme is expressed, such as a β-1,3-N-acetyl-glucosaminyl-transferase, e.g., LgtA from Neisseria meningitidis.

In embodiments the cell further comprises one or more recombinant nucleic acid sequence encoding a α-2,3-sialyltransferase. Exemplified further glycosyltransferases in addition to the α-1,3-fucosyltransferases, Osc1, Bgall1, Murba1, BAcfin1, Bbac1, Prev1, Csec1, CafC, FutA_mut2 and FucT109 are preferably selected from the glycosyltransferases described below (tables 1, 2, 3 and 9).

α-1,3-fucosyltransferase

The term “α-1,3-fucosyltransferase” refers to a glycosyltransferase that catalyzes the transfer of fucosyl from a donor substrate, such as GDP-fucose, to an acceptor molecule in an α-1,3-linkage (see FIG. 1). Preferably, an α-1,3-fucosyltransferase used in the present invention does not originate in the species of the genetically engineered cell, i.e., the gene encoding the α-1,3-fucosyltransferase is of heterologous origin and is selected from an α-1,3-fucosyltransferase identified in table 1. In the context described herein, the acceptor molecule for the α-1,3-fucosyltransferase is preferably an acceptor oligosaccharide of at least four monosaccharide units with a GlcNAc moiety, e.g., LNnT. Heterologous α-1,3-fucosyltransferases that are capable of transferring a fucosyl moiety onto LNnT are known in the art, specifically FutA has been shown to produce a mixture of LNFP-VI and LNDFH-III (Dumon et al 2004 Biotechnol. Prog. 20:412-419).

The α-1,3-fucosyltransferase can be selected from an amino acid sequence with at least 80%, such as at least 85%, such as at least 90%, such as at least 95%, or such as at least 99% sequence identity to the amino acid sequence of any one of the α-1,3-fucosyltransferases listed in table 1.

TABLE 1
List of α-1,3(4)-fucosyltransferase enzymes capable
of producing LNDFH-III and mixtures comprising LNDFH-III.

| | | SEQ | |
|---|---|---|---|
| Name | GenBank ID¹ | ID NO | Origin |
| Osc1 | MBC8592593.1 | 1 | Oscillospiraceae bacterium |
| | | | N12 |
| Bgall1 | WP_204430034.1 | 2 | Bacteroides gallinaceum |
| Bbac1 | MBR5851405.1 | 3 | Bacteroidaceae bacterium |
| Murba1 | MBP3303338.1 | 4 | Muribaculaceae bacterium |
| Bacfin1 | WP_195630400.1 | 5 | Bacteroides finegoldii |
| Prev1 | MBP3220331.1 | 6 | Prevotella sp. |
| Csec1 | KHM48694.1 | 7 | Coprobacter secundus |
| CafC | WP_007483358.1 | 8 | Bacteroides nordii |
| FutA_{—} | — | 9 | synthetic construct |
| mut2 | | | |
| FucT109 | CAH09151.1 | 10 | Bacteroides fragilis |
| | | | NCTC 9343 |
| FutA | WP_000487428.1 | 11 | Helicobacter pylori |

¹The GenBank IDs reflect the full-length enzymes, in the present invention truncated, elongated or mutated versions may have been used, these are represented by the sequences indicated by the SEQ ID NOs.

Example 1 discloses the identification of the heterologous α-1,3-fucosyltransferases Osc1, Bgall1, Murba1, Bacfin1, Prev1, Csec1, Cafc, FutA_mut2, FucT109 and FutA (SEQ ID NO: 1, 2, 3, 4, 5, 6, 7, 8, 9, 10 and 11, respectively), which are each capable of producing mixtures of HMOs comprising LNDFH-III when introduced into an LNnT producing cell. Specifically, the enzymes Osc1, Bgall1, Murba1, Bacfin1, Prev1, Csec1, CafC, FutA_mut2, FucT109 and FutA can transfer a fucosyl unit onto the Glc moiety of LNnT and/or LNFP-III in an α-1,3 linkage to form LNFP-VI and/or LNDFH-III, respectively, and/or onto the GlcNAc moiety of LNnT in an α-1,3 linkage to form LNFP-III and/or LNDFH-III, respectively (see FIG. 1).

Furthermore, the experiments performed in Example 1 show that the enzymes Murba1, Bgall1, Bacfin1 and FutA_mut2 do not produce any LNFP-VI, or at least not any detectable amount of LNFP-VI, or at least below 1% LNFP-VI of the total amount of HMO produced, as a final product. In addition, the experiments performed in Example 1 show that the enzymes FutA and CafC do not produce any LNFP-III, or at least not any detectable amount of LNFP-III.

A fucosyl transferase described herein is capable of transferring a fucosyl moiety from a fucosyl donor to an acceptor oligosaccharide in an α-1,3 linkage. Such an enzyme is also known as an α-1,3-fucosyltransferase. In preferred embodiments the α-1,3-fucosyltransferase possesses dual α-1,3-fucosyltransferase activity, meaning it is capable of fucosylating an oligosaccharide at a GlcNAc moiety and a Glc moiety.

In embodiments, the α-1,3-fucosyltransferase is selected from the group consisting of a) Osc1 comprising or consisting of the amino acid sequence of SEQ ID NO: 1, or a functional homologue thereof with an amino acid sequence that is at least 80% identical to SEQ ID NO: 1,b) Bgall1 comprising or consisting of the amino acid sequence of SEQ ID NO: 2, or a functional homologue thereof with an amino acid sequence that is at least 80% identical to SEQ ID NO: 2, c) Bbac1 comprising or consisting of the amino acid sequence of SEQ ID NO: 3, or a functional homologue thereof with an amino acid sequence that is at least 80% identical to SEQ ID NO: 3, d) Murba1 comprising or consisting of the amino acid sequence of SEQ ID NO: 4, or a functional homologue thereof with an amino acid sequence that is at least 80% identical to SEQ ID NO: 4, e) Bacfin1 comprising or consisting of the amino acid sequence of SEQ ID NO: 5, or a functional homologue thereof with an amino acid sequence that is at least 80% identical to SEQ ID NO: 5, f) Prev1 comprising or consisting of the amino acid sequence of SEQ ID NO: 6, or a functional homologue thereof with an amino acid sequence that is at least 80% identical to SEQ ID NO: 6, g) Csec1 comprising or consisting of the amino acid sequence of SEQ ID NO: 7, or a functional homologue thereof with an amino acid sequence that is at least 80% identical to SEQ ID NO: 7, h) CafC comprising or consisting of the amino acid sequence of SEQ ID NO: 8, or a functional homologue thereof with an amino acid sequence that is at least 80% identical to SEQ ID NO: 8, i) FutA_mut2 comprising or consisting of the amino acid sequence of SEQ ID NO: 9, or a functional homologue thereof with an amino acid sequence that is at least 80% identical to SEQ ID NO: 9, j) FutA variants comprising substitutions at a position corresponding to position 128 and 129 of SEQ ID NO: 11, wherein the variants have at least 80% sequence identity to SEQ ID NO: 11 and k) FucT109 comprising or consisting of the amino acid sequence of SEQ ID NO: 10, or a functional homologue thereof with an amino acid sequence that is at least 80% identical to SEQ ID NO: 10.

In preferred embodiments the α-1,3-fucosyltransferase is selected from the group consisting of a) Osc1 comprising or consisting of the amino acid sequence of SEQ ID NO: 1, or a functional homologue thereof with an amino acid sequence that is at least 80% identical to SEQ ID NO: 1, b) Bgall1 comprising or consisting of the amino acid sequence of SEQ ID NO: 2, or a functional homologue thereof with an amino acid sequence that is at least 80% identical to SEQ ID NO: 2, c) Bbac1 comprising or consisting of the amino acid sequence of SEQ ID NO: 3, or a functional homologue thereof with an amino acid sequence that is at least 80% identical to SEQ ID NO: 3. These enzymes can e.g., be used to produce LNDFH-Ill with low levels of LNFP-III and LNFP-VI, such as less than 10% of the total HMO of each. These enzymes can further be used to produce HMO mixtures LNDFH-III and 3FL, where these two HMOs constitute at least 80%, such as at least 90% of the total HMO produced.

In other preferred embodiments the α-1,3-fucosyltransferase capable of transferring a fucosyl moiety from a fucosyl donor to an acceptor oligosaccharide is selected from the group consisting of a) Bgall1 comprising or consisting of the amino acid sequence of SEQ ID NO: 2, or a functional homologue thereof with an amino acid sequence that is at least 80% identical to SEQ ID NO: 2, b) Bacfin1 comprising or consisting of the amino acid sequence of SEQ ID NO: 5, or a functional homologue thereof with an amino acid sequence that is at least 80% identical to SEQ ID NO: 5, c) Murba1 comprising or consisting of the amino acid sequence of SEQ ID NO: 4, or a functional homologue thereof with an amino acid sequence that is at least 80% identical to SEQ ID NO: 4 and d) FutA variants comprising substitutions at a position corresponding to position 128 and 129 of SEQ ID NO: 11, wherein the variants have at least 80% sequence identity to SEQ ID NO: 11. These enzymes can e.g., be used to produce HMO mixtures comprising LNFP-III, and LNDFH-III, with LNFP-VI constituting less the less than 1% of the total HMO produced.

In other preferred embodiments the α-1,3-fucosyltransferase capable of transferring a fucosyl moiety from a fucosyl donor to an acceptor oligosaccharide is selected from the group consisting of a) Bbac1 comprising or consisting of the amino acid sequence of SEQ ID NO: 3, or a functional homologue thereof with an amino acid sequence that is at least 80% identical to SEQ ID NO: 3, b) Prev1 comprising or consisting of the amino acid sequence of SEQ ID NO: 6, or a functional homologue thereof with an amino acid sequence that is at least 80% identical to SEQ ID NO: 6, c) FucT109 comprising or consisting of the amino acid sequence of SEQ ID NO: 10, or a functional homologue thereof with an amino acid sequence that is at least 80% identical to SEQ ID NO: 10. d) Csec1 comprising or consisting of the amino acid sequence of SEQ ID NO: 7, or a functional homologue thereof with an amino acid sequence that is at least 80% identical to SEQ ID NO: 7. These enzymes can e.g., be used to produce HMO mixtures comprising LNDFH-III and LNFP-III and LNFP-VI.

In one embodiment, the α-1,3-fucosyltransferase is Osc1 from Oscillospiraceae bacterium N12 comprising or consisting of the amino acid sequence of SEQ ID NO: 1, or an amino acid sequence with at least 80%, such as at least 85%, such as at least 90%, such as at least 95%, or such as at least 99% sequence identity to SEQ ID NO: 1.

In one embodiment, the α-1,3-fucosyltransferase is Bgall1 from Bacteroides gallinaceum comprising or consisting of the amino acid sequence of SEQ ID NO: 2, or an amino acid sequence with at least 80%, such as at least 85%, such as at least 90%, such as at least 95%, or such as at least 99% sequence identity to SEQ ID NO: 2.

In one embodiment, the α-1,3-fucosyltransferase is Bbac1 from Bacteroidaceae bacterium comprising or consisting of the amino acid sequence of SEQ ID NO: 3, or an amino acid sequence with at least 80%, such as at least 85%, such as at least 90%, such as at least 95%, or such as at least 99% sequence identity to SEQ ID NO: 3.

In one embodiment, the α-1,3-fucosyltransferase is Murba1 from Muribaculaceae bacterium comprising or consisting of the amino acid sequence of SEQ ID NO: 4, or an amino acid sequence with at least 80%, such as at least 85%, such as at least 90%, such as at least 95%, or such as at least 99% sequence identity to SEQ ID NO: 4.

In one embodiment, the α-1,3-fucosyltransferase is Bacfin1 from Bacteroides finegoldii comprising or consisting of the amino acid sequence of SEQ ID NO: 5, or an amino acid sequence with at least 80%, such as at least 85%, such as at least 90%, such as at least 95%, or such as at least 99% sequence identity to SEQ ID NO: 5.

In one embodiment, the α-1,3-fucosyltransferase is Prev1 from Prevotella sp. comprising or consisting of the amino acid sequence of SEQ ID NO: 6, or an amino acid sequence with at least 80%, such as at least 85%, such as at least 90%, such as at least 95%, or such as at least 99% sequence identity to SEQ ID NO: 6.

In one embodiment, the α-1,3-fucosyltransferase is Csec1 from Coprobacter secundus comprising or consisting of the amino acid sequence of SEQ ID NO: 7, or an amino acid sequence with at least 80%, such as at least 85%, such as at least 90%, such as at least 95%, or such as at least 99% sequence identity to SEQ ID NO: 7.

In one embodiment, the α-1,3-fucosyltransferase is CafC from Bacteroides nordii comprising or consisting of the amino acid sequence of SEQ ID NO: 8, or an amino acid sequence with at least 80%, such as at least 85%, such as at least 90%, such as at least 95%, or such as at least 99% sequence identity to SEQ ID NO: 8.

In one embodiment, the α-1,3-fucosyltransferase is FucT109 from Bacteroides fragilis NCTC 9343 comprising or consisting of the amino acid sequence of SEQ ID NO: 10, or an amino acid sequence with at least 80%, such as at least 85%, such as at least 90%, such as at least 95%, or such as at least 99% sequence identity to SEQ ID NO: 10.

In one embodiment, the α-1,3-fucosyltransferase is FutA from Helicobacter pylori comprising or consisting of the amino acid sequence of SEQ ID NO: 11, or an amino acid sequence with at least 80%, such as at least 85%, such as at least 90%, such as at least 95%, or such as at least 99% sequence identity to SEQ ID NO: 11.

In one embodiment, the α-1,3-fucosyltransferase is a FutA variants comprising substitutions at a position corresponding to position 128 and 129 of SEQ ID NO: 11, wherein the variants have at least 80%, such as at least 85%, such as at least 90%, such as at least 95%, or such as 99.5% sequence identity to SEQ ID NO: 11.

In one embodiment, the α-1,3-fucosyltransferase is the FutA variant, FutA_mut2, comprising or consisting of the amino acid sequence of SEQ ID NO: 9.

In one embodiment, the enzyme Osc1 is introduced into a genetically engineered cell which further comprises a β-1,4-galactosyltransferase and preferably also a β-1,3-N-acetyl-glucosaminyltransferase.

In one embodiment, the enzyme Murba1 is introduced into a genetically engineered cell which further comprises a β-1,4-galactosyltransferase and preferably also a β-1,3-N-acetyl-glucosaminyl-transferase.

In one embodiment, the enzyme Csec1 is introduced into a genetically engineered cell which further comprises a β-1,4-galactosyltransferase and preferably also a β-1,3-N-acetyl-glucosaminyl-transferase.

In one embodiment, the enzyme Bbac1 is introduced into a genetically engineered cell which further comprises a β-1,4-galactosyltransferase and preferably also a β-1,3-N-acetyl-glucosaminyl-transferase.

In one embodiment, the enzyme Bacfin1 is introduced into a genetically engineered cell which further comprises a β-1,4-galactosyltransferase and preferably also a β-1,3-N-acetyl-glucosaminyl-transferase.

In one embodiment, the enzyme Prev1 is introduced into a genetically engineered cell which further comprises a β-1,4-galactosyltransferase and preferably also a β-1,3-N-acetyl-glucosaminyl-transferase.

In one embodiment, the enzyme Bgall1 is introduced into a genetically engineered cell which further comprises a β-1,4-galactosyltransferase and preferably also a β-1,3-N-acetyl-glucosaminyl-transferase.

In one embodiment, the enzyme CafC is introduced into a genetically engineered cell which further comprises a β-1,4-galactosyltransferase and preferably also a β-1,3-N-acetyl-glucosaminyl-transferase. In one embodiment, the enzyme FutA_mut2 is introduced into a genetically engineered cell which further comprises a β-1,4-galactosyltransferase and preferably also a β-1,3-N-acetyl-glucosaminyl-transferase.

In one embodiment, the enzyme FucT109 is introduced into a genetically engineered cell which further comprises a β-1,4-galactosyltransferase and preferably also a β-1,3-N-acetyl-glucosaminyl-transferase.

In one embodiment, the enzyme FutA is introduced into a genetically engineered cell which further comprises a β-1,4-galactosyltransferase and preferably also a β-1,3-N-acetyl-glucosaminyl-transferase.

β-1,3-N-acetyl-glucosaminyl-transferase

A β-1,3-N-acetyl-glucosaminyl-transferase is any protein which comprises the ability of transferring the N-acetyl-glucosamine of UDP-N-acetyl-glucosamine to lactose or another acceptor molecule, in a beta-1,3-linkage (see FIG. 1). Preferably the β-1,3-N-acetyl-glucosaminyl-transferase used herein does not originate in the species of the genetically engineered cell, i.e., the gene encoding the β-1,3-N-acetyl-glucosaminyl-transferase is of heterologous origin.

Accordingly, in embodiments, the genetically engineered cell further comprises one or more recombinant nucleic acid sequence(s) encoding a β-1,3-N-acetyl-glucosaminyltransferase.

Non-limiting examples of β-1,3-N-acetyl-glucosaminyltransferases are given in table 2. β-1,3-N-acetyl-glucosaminyltransferase variants may also be useful, preferably such variants are at least 80%, such as at least 85%, such as at least 90%, such as at least 95% identical to the amino acid sequence of any one of the β-1,3-N-acetyl-glucosaminyltransferase in table 2.

TABLE 2
List of β-1,3-N-acetyl-glucosaminyltransferase

| Protein Name | GenBank ID | origin |
|---|---|---|
| IgtA | WP_033911473.1 | Neisseria meningitidis |
| | WP_002248149 | |
| | AAF42258.1 | |
| | ELK60643.1 | |
| IgtA | AAK70338.1 | Neisseria gonorrhoeae |
| | ACF31229.1 | |
| IgtA | AAK02595.1 | Pasteurella multocida |
| IgtA | EEZ72046.1 | Neisseria cinerea |
| HD0466 | WP_010944479.1 | Haemophilus ducreyi |
| PmnagT | WP_014390683.1 | Pasteurella multocida |

In embodiments, the genetically engineered cell comprises a recombinant nucleic acid sequence encoding a β-1,3-N-acetyl-glucosaminyltransferase. In one embodiment, the recombinant nucleic acid sequence encoding a β-1,3-N-acetylglucosaminyltransferase comprises or consists of the amino acid sequence of SEQ ID NO: 24 (LgtA from N. meningitidis) or a functional homologue thereof with an amino acid sequence with at least 80%, such as at least 85%, such as at least 90%, such as at least 95%, or such as at least 99% sequence identity to SEQ ID NO: 24.

For the production of LNnT from lactose as substrate, the LNT-II precursor is formed using a β-1,3-N-acetylglucosaminyltransferase. In one embodiment the genetically engineered cell comprises a β-1,3-N-acetylglucosaminyltransferase gene, or a functional homologue or fragment thereof, to produce the intermediate LNT-II from lactose.

Some of the examples below use the heterologous β-1,3-N-acetyl-glucosaminyl-transferase named LgtA from Neisseria meningitidis or a variant thereof.

β-1,4-galactosyltransferase

A β-1,4-galactosyltransferase is any protein that comprises the ability of transferring the galactose of UDP-Galactose to a N-acetyl-glucosaminyl moiety to an acceptor molecule in a β-1,4-linkage (see FIG. 1). Preferably, a β-1,4-galactosyltransferase used herein does not originate in the species of the genetically engineered cell i.e., the gene encoding the β-1,4-galactosyltransferase is of heterologous origin. In the context described herein the acceptor molecule, is an acceptor saccharide, e.g., LNT-II, or more complex HMO structures.

The examples below use the heterologous β-1,4-galactosyltransferase GalT, or a variant thereof, to produce LNnT e.g., and in in combination with α-1,3-fucosyltransferase described herein it can produce LNFP-III, LNFP-VI and/or LNDFH-II. Accordingly, in embodiments, the genetically engineered cell comprises one or more recombinant nucleic acid sequence(s) encoding a β-1,4-galactosyltransferase.

Non-limiting examples of 3-1,4-galactosyltransferases are provided in table 2. β-1,4-galactosyltransferases variants may also be useful, preferably such variants are at least 80%, such as at least 85%, such as at least 90, such as at least 95% identical to the amino acid sequence of any one of the β-1,4-galactosyltransferases in table 3.

TABLE 3
List of β-1,4-glycosyltransferases

| Protein Name | GenBank ID | Origin |
|---|---|---|
| GalT | WP_001262061.1 | Helicobacter pylori |
| LgtB | AAF42257.1 | Neisseria meningitidis MC58 |

In embodiments described herein the β-1,3-N-acetylglucosaminyltransferase is from Neisseria meningitidis, and the β-1,3-galactosyltransferase and/or 3-1,4-galactosyltransferase is from Helicobacter pylori from Helicobacter pylori, respectively.

In one embodiment, the recombinant nucleic acid sequence encoding a β-1,4-galactosyltransferases comprises or consists of the amino acid sequence of SEQ ID NO: 25 (galT from H. pylori) or a functional homologue thereof with an amino acid sequence with at least 80%, such as at least 85%, such as at least 90%, such as at least 95%, or such as at least 99% sequence identity to SEQ ID NO: 25.

To produce LNnT form an LNT-II precursor, a β-1,4-galactosyltransferase is needed. In one embodiment, the genetically engineered cell comprises a β-1,4-galactosyltransferase gene, or a functional homologue or fragment thereof. In embodiments, the β-1,3-N-acetylglucosaminyltransferase is from Neisseria meningitidis and the β-1,4-galactosyltransferase is from Helicobacter pylori. In further embodiments, the β1,3-N-acetylglucosaminyltransferase has an amino acid sequence according to SEQ ID NO: 24, or a functional homologue thereof with an amino acid sequence that is at least 80% identical to SEQ ID NO: 24 and the β-1,4-galactosyltransferase has an amino acid sequence according to SEQ ID NO: 25, or a functional homologue thereof with an amino acid sequence that is at least 80% identical to SEQ ID NO: 25.

α-2,3-sialyltransferase

An α-2,3-sialyltransferase refers to a glycosyltransferase that catalyzes the transfer of a sialyl moiety from a donor substrate, such as CMP-N-acetylneuraminic acid, to an acceptor molecule e.g., lactose or LNnT in an α-2,3-linkage. Preferably, an α-2,3-sialyltransferase used herein does not originate in the species of the genetically engineered cell, i.e., the gene encoding the α-2,3-sialyltransferase is of heterologous origin and is selected from an α-2,3-sialyltransferase identified in table 9. Heterologous α 2,3-sialyltransferases that are capable of transferring a sialyl moiety onto lactose are known in the art, three of which are identified in table 9.

The sialyltransferase can be selected from an amino acid sequence with at least 80%, such as 80%, such as at least 90%, such as at least 95%, or such as at least 99% identity to the amino acid sequence of any one of the α-2,3-sialyltransferases listed in table 1.

TABLE 9
List of α-2,3-sialyltransferase enzymes capable of producing 3′SL.

| Enzyme Name | GenBank ID | Origin |
|---|---|---|
| Clari1 | EGK8106227.1 | Campylobacter lari |
| Neigon | AAW89748.1 | Neisseria gonorrhoeae FA 1090 |
| Poral | WP_101774487.1 | Pasteurella oralis |
| PmN | WP_005726268.1 | Pasteurella (multispecies) |
| Nst | AAC44541.1 | Neisseria meningitidis MC58 |
| PM70 | AAK03258.1 | Pasteurella multocida subsp. Pm70 |
| Pmult | WP_005753497.1 | Pasteurella multocida |
| Avi | WP_115249238.1 | Avibacterium avium |
| MhnN | WP_176810284.1 | Mannheimia (multispecies) |
| Csub1 | WP_039664428.1 | Campylobacter subantarcticus |
| Ccol | WP_075498955.1 | Campylobacter coli |
| Cstl | AAF13495.1 | Campylobacter jejuni |

In one embodiment, the enzyme Osc1 is introduced into a genetically engineered cell which further comprises an α-1,3-sialyltransferase, wherein the cell is capable of producing FSL, such as a mixture of 3′SL, 3FL and FSL.

In one embodiment, the enzyme FucT109 is introduced into a genetically engineered cell which further comprises an α-2,3-sialyltransferase, wherein the cell is capable of producing FSL, such as a mixture of 3′SL, 3FL and FSL.

In one embodiment, the enzyme Murba1 is introduced into a genetically engineered cell which further comprises an α-2,3-sialyltransferase, wherein the cell is capable of producing FSL, such as a mixture of 3′SL, 3FL and FSL.

In one embodiment, the enzyme Bgall1 is introduced into a genetically engineered cell which further comprises an α-2,3-sialyltransferase, wherein the cell is capable of producing FSL, such as a mixture of 3′SL, 3FL and FSL.

In one embodiment, the enzyme CafC is introduced into a genetically engineered cell which further comprises an α-2,3-sialyltransferase, wherein the cell is capable of producing FSL, such as a mixture of 3′SL, 3FL and FSL.

In preferred embodiments the α-2,3-sialyltransferase is Clari1 from Campylobacter lari (GenBank protein Accession No. EGK8106227.1), or Poral from Pasteurella oralis (GenBank protein Accession No. WP_101774487.1).

### Glycosyl-Donor—Nucleotide-Activated Sugar Pathways

When carrying out the method of this invention, preferably a glycosyltransferase mediated glycosylation reaction takes place in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably the following activated sugar nucleotides are involved in the glycosyl transfer: glucose-UDP-GlcNAc, UDP-galactose, UDP-glucose, UDP-N-acetylglucosamine, UDP-N-acetylgalactosamine (GlcNAc) and CMP-N-acetylneuraminic acid.

The genetically engineered cell according to the present invention can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid.

In one embodiment of the current invention, the genetically engineered cell is capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).

The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically engineered cell can utilize salvaged monosaccharides for sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cell.

### Colanic Acid Gene Cluster

For the production of fucosylated HMOs, the de novo GDP-fucose pathway is important to ensure presence of sufficient GDP-fucose. The colanic acid gene cluster of Escherichia coli encodes selected enzymes involved in the de novo synthesis of GDP-fucose (gmd, wcaG, wcaH, wcal, manB, manC), whereas one or several of the genes downstream of GDP-L-fucose such as wcaJ, which are responsible for the production of the extracellular polysaccharide colanic acid, a major oligosaccharide of the bacterial cell wall, can be deleted to prevent conversion of GDP-fucose to colanic acid.

To secure sufficient amounts of GDP-fucose the promoter of the native colanic acid gene cluster may be exchanged with a stronger promoter, generating a recombinant colanic acid gene cluster, to drive additional production of GDP-fucose. Furthermore, an extra copy of the colanic acid gene cluster or selected genes thereof can be introduced in the genetically engineered cells as described in the examples.

In embodiments, the colanic acid gene cluster may be expressed from its native genomic locus. The expression may be actively modulated. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

In relation to the present disclosure, the term “native genomic locus”, in relation to the colanic acid gene cluster, relates to the original and natural position of the gene cluster in the genome of the genetically engineered cell.

The de novo GDP-fucose pathway genes responsible for the formation of GDP-fucose comprises or consists of the following genes:
i) manA which encodes the protein mannose-6 phosphate isomerase (EC 5.3.1.8, UniProt accession nr. P00946), which facilitates the interconversion of fructose 6-phosphate (F6P) and mannose-6-phosphate;
ii) manB which encodes the protein phosphomannomutase (EC 5.4.2.8, UniProt accession nr P24175), which is involved in the biosynthesis of GDP-mannose by catalyzing conversion mannose-6-phosphate into mannose-1-phosphate;
iii) manC which encodes the protein mannose-1-phosphate guanylyltransferase guanylyltransferase (EC: 2.7.7.13, UniProt accession nr P24174), which is involved in the biosynthesis of GDP-mannose through synthesis of GDP-mannose from GTP and α-D-mannose-1-phosphate;
iv) gmd which encodes the protein GDP-mannose-4,6-dehydratase (UniProt accession nr P0AC88), which catalyzes the conversion of GDP-mannose to GDP-4-dehydro-6-deoxy-D-mannose;
v) wcaG (fcl) which encodes the protein GDP-L-fucose synthase (EC 1.1.1.271, UniProt accession nr P32055) which catalyses the two-step NADP-dependent conversion of GDP-4-dehydro-6-deoxy-D-mannose to GDP-fucose.

Accordingly, it is preferred that the genetically engineered cell, when producing one or more fucosylated heterologous products, overexpresses either the entire colonic acid gene cluster and/or one or more genes of the de novo GDP-fucose pathway selected from the group consisting of manA, manB, manC, gmd and wcaG.

### Lactose Permease

Lactose permease is a membrane protein which is a member of the major facilitator superfamily and can be classified as a symporter, which uses the proton gradient towards the cell to transport β-galactosides such as lactose in the same direction into the cell. In oligosaccharide-production, especially in the production of human milk oligosaccharides (HMOs), lactose is often the initial substrate being decorated to produce any HMO of interest in a bioconversion that happens in the cell interior. Thus, in the production of HMOs, there is a desire to be able to import lactose into the cell, e.g., by expression and/or overexpression of a lactose permease such as lacY of E. coli.

In embodiments, the lactose permease is as shown in SEQ ID NO: 26, or a functional homologue thereof having an amino acid sequence which is at least 80% identical, such as at least 85%, 90% or 95% identical to SEQ ID NO: 26.

In embodiments, the expression of the lactose permease is regulated by a promoter according to the present invention.

### β-Galactosidase

A host cell suitable for HMO production, e.g., E. coli, may comprise an endogenous β-galactosidase gene or an exogenous β-galactosidase gene, e.g., E. coli comprises an endogenous lacZ gene (e.g., GenBank Accession Number V00296 (GI: 41901)). For the purposes of the invention, when producing an HMO, it is preferred that the genetically engineered cell does not express a functional β-galactosidase to avoid the degradation of lactose if lactose is used as the initial substrate for producing the complex fucosylated HMO.

In embodiments the lacZ gene may be inactivated by a complete or partial deletion of the corresponding nucleic acid sequence from the bacterial genome, or the gene sequence is mutated in the way that it is not transcribed, or, if transcribed, the transcript is not translated or if translated to a protein (i.e., β-galactosidase), the protein does not have the corresponding enzymatic activity. In this way the HMO-producing bacterium accumulates an increased intracellular lactose pool which is beneficial for the production of HMOs.

### Importer Proteins

Most commonly HMO producing cells are genetically engineered to use lactose as the initial substrate since this is easily taken up by lactose permease as described above. However, it may be desired to use an initial substrate that will require the presence of fewer glycosyltransferases in the cell, since this will reduce the strain on the cell in terms of producing multiple enzymes and in addition it can reduce the by-product profile, e.g. if lactose is not used as initial substrate a cell comprising a fucosyltransferase will not produce 3FL as by-product allowing the fucose to be used to produce e.g. more LNFP-V and LNDFH-II.

Examples of suitable LNT-II and LNnT importers are described in WO2023099680 and includes for example,
Lactose permease (LacY) mutants, such as LacY mutant Y236H or LacY mutant A177V+S306T, wherein the mutations are equivalent with the corresponding position in the sequence of SEQ ID NO: 14,
ABC transporter protein complexes, such as ABC transporter from B. pseudocatenulatum JCM 1200 BBPC_1775, 1776, 1777, (NCBI accession Nrs BAR04453.1, BAR04454.1 and BAR04455.1, respectively) or ABC transporter from B. breve UCC2003 BBR_0527/IntP1, BBR_0528/IntP2, BBR_0530/IntS and BBR_0531 (NCBI accession Nrs ABE95224.1, ABE95225.1, ABE95226.1 and ABE95228.1), and/or
MFS transporters, such as but not limited to Blon_0962 (NCBI accession Nr ACJ52061.1).

Accordingly, in embodiments, a nucleic acid or a cluster of nucleic acids encoding one of these transporters may be introduced into a genetically modified cell as described herein. The expression of such transporters enables the production of complex fucosylated oligosaccharide with LNT-II as the initial substrate.

### Exporter Proteins

The oligosaccharide product, such as the HMO produced by the cell, can be accumulated both in the intra- and the extracellular matrix. The product can be transported to the supernatant in a passive way, i.e., it diffuses outside across the cell membrane. The more complex HMO products may remain in the cell, which is likely to eventually impair cellular growth, thereby affecting the possible total yield of the product from a single fermentation. The HMO transport can be facilitated by major facilitator superfamily transporter proteins that promote the effluence of sugar derivatives from the cell to the supernatant. The exporter can be present exogenously or endogenously and is overexpressed under the conditions of the fermentation to enhance the export of the oligosaccharide derivative (HMO) produced. The specificity towards the oligosaccharide product to be secreted can be altered by mutation by means of known recombinant DNA techniques.

Thus, the genetically engineered cell according to the present invention can further comprise a nucleic acid sequence encoding an exporter protein capable of exporting the fucosylated human milk oligosaccharide product or products, such as transporter protein can for example be a member of the major facilitator superfamily transport proteins.

In the resent years, several new and efficient major facilitator superfamily transporter proteins have been identified as exporters of HMOs, each having specificity for different recombinantly produced HMOs and development of recombinant cells expressing said proteins are advantageous for high scale industrial HMO manufacturing.

### The Genetically Engineered Cell

In the present context, the terms “a genetically engineered cell” and “a genetically modified cell” are used interchangeably. As used herein “a genetically engineered cell” is a host cell whose genetic material has been altered by human intervention using a genetic engineering technique, such a technique is e.g., but not limited to transformation or transfection e.g., with a heterologous and/or recombinant polynucleotide sequence, Crisper/Cas editing and/or random mutagenesis. In one embodiment the genetically engineered cell has been transformed or transfected with a recombinant nucleic acid sequence.

The genetic modifications can e.g., be selected from inclusion of glycosyltransferases, and/or metabolic pathway engineering deletion of repressors or undesired enzymes and inclusion of transporters as described in the above sections, which the skilled person will know how to combine into a genetically engineered cell capable of producing one or more fucosylated HMO's.

In one embodiment the genetically engineered cell capable of producing LNDFH-III, comprises a recombinant nucleic acid sequence encoding an α-1,3-fucosyltransferase selected from the group consisting of a) Osc1 comprising or consisting of the amino acid sequence of SEQ ID NO: 1, or a functional homologue thereof with an amino acid sequence that is at least 80% identical to SEQ ID NO: 1, b) Bgall1 comprising or consisting of the amino acid sequence of SEQ ID NO: 2, or a functional homologue thereof with an amino acid sequence that is at least 80% identical to SEQ ID NO: 2, c) Bbac1 comprising or consisting of the amino acid sequence of SEQ ID NO: 3, or a functional homologue thereof with an amino acid sequence that is at least 80% identical to SEQ ID NO: 3, d) Murba1 comprising or consisting of the amino acid sequence of SEQ ID NO: 4, or a functional homologue thereof with an amino acid sequence that is at least 80% identical to SEQ ID NO: 4, e) Bacfin1 comprising or consisting of the amino acid sequence of SEQ ID NO: 5, or a functional homologue thereof with an amino acid sequence that is at least 80% identical to SEQ ID NO: 5, f) Prev1 comprising or consisting of the amino acid sequence of SEQ ID NO: 6, or a functional homologue thereof with an amino acid sequence that is at least 80% identical to SEQ ID NO: 6, g) Csec1 comprising or consisting of the amino acid sequence of SEQ ID NO: 7, or a functional homologue thereof with an amino acid sequence that is at least 80% identical to SEQ ID NO: 7, h) CafC comprising or consisting of the amino acid sequence of SEQ ID NO: 8, or a functional homologue thereof with an amino acid sequence that is at least 80% identical to SEQ ID NO: 8, i) FutA variants comprising substitutions at a position corresponding to position 128 and 129 of SEQ ID NO: 11, wherein the variants have at least 80% sequence identity to SEQ ID NO: 11, j) futA_mut2 comprising or consisting of the amino acid sequence of SEQ ID NO: 9, or a functional homologue thereof with an amino acid sequence that is at least 80% identical to SEQ ID NO: 9, k) FucT109 comprising or consisting of the amino acid sequence of SEQ ID NO: 10, or a functional homologue thereof with an amino acid sequence that is at least 80% identical to SEQ ID NO: 10.

Preferably, the fucosyltransferases have α-1,3-fucosyltransferase activity, allowing fucosylation of an oligosaccharide at position 3 of a GlcNAc moiety and at position 3 of a Glc moiety, while showing limited or no fucosylation at position 2 of the Gal moiety. Preferably, the Glc moiety is at the reducing end of the oligosaccharide, more preferably the oligosaccharide is LNnT. In one embodiment the genetically engineered cell capable of producing LNDFH-III, comprises a recombinant nucleic acid sequence encoding an α-1,3-fucosyltransferase selected from the group consisting of a) Osc1 comprising or consisting of the amino acid sequence of SEQ ID NO: 1, or a functional homologue thereof with an amino acid sequence that is at least 80% identical to SEQ ID NO: 1, b) Bgall1 comprising or consisting of the amino acid sequence of SEQ ID NO: 2, or a functional homologue thereof with an amino acid sequence that is at least 80% identical to SEQ ID NO: 2, c) Bbac1 comprising or consisting of the amino acid sequence of SEQ ID NO: 3, or a functional homologue thereof with an amino acid sequence that is at least 80% identical to SEQ ID NO: 3. These enzymes can e.g., be used to produce LNDFH-III with low levels of LNFP-III and LNFP-VI, such as less than 10% of the total HMO of each.

Preferably the genetically engineered cell expressing these enzymes produce HMO mixtures LNDFH-III and 3FL, where these two HMOs constitute at least 80%, such as at least 90% of the total HMO produced.

In one embodiment the genetically engineered cell capable of producing LNDFH-III, comprises a recombinant nucleic acid sequence encoding an α-1,3-fucosyltransferase selected from the group consisting of a) Bgall1 comprising or consisting of the amino acid sequence of SEQ ID NO: 2, or a functional homologue thereof with an amino acid sequence that is at least 80% identical to SEQ ID NO: 2, b) Bacfin1 comprising or consisting of the amino acid sequence of SEQ ID NO: 5, or a functional homologue thereof with an amino acid sequence that is at least 80% identical to SEQ ID NO: 5, c) Murba1 comprising or consisting of the amino acid sequence of SEQ ID NO: 4, or a functional homologue thereof with an amino acid sequence that is at least 80% identical to SEQ ID NO: 4 and d) FutA variants comprising substitutions at a position corresponding to position 128 and 129 of SEQ ID NO: 11, wherein the variants have at least 80% sequence identity to SEQ ID NO: 11.

Preferably the genetically engineered cell expressing these enzymes produce HMO mixtures comprising LNFP-III, and LNDFH-III, with LNFP-VI constituting less the less than 1% of the total HMO produced.

In one embodiment the genetically engineered cell capable of producing LNDFH-III, comprises a recombinant nucleic acid sequence encoding an α-1,3-fucosyltransferase selected from the group consisting of a) Bbac1 comprising or consisting of the amino acid sequence of SEQ ID NO: 3, or a functional homologue thereof with an amino acid sequence that is at least 80% identical to SEQ ID NO: 3, b) Prev1 comprising or consisting of the amino acid sequence of SEQ ID NO: 6, or a functional homologue thereof with an amino acid sequence that is at least 80% identical to SEQ ID NO: 6, c) FucT109 comprising or consisting of the amino acid sequence of SEQ ID NO: 10, or a functional homologue thereof with an amino acid sequence that is at least 80% identical to SEQ ID NO: 10. d) Csec1 comprising or consisting of the amino acid sequence of SEQ ID NO: 7, or a functional homologue thereof with an amino acid sequence that is at least 80% identical to SEQ ID NO: 7.

Preferably the genetically engineered cell expressing these enzymes produce HMO mixtures comprising LNDFH-III and LNFP-III and LNFP-VI.

In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is Osc1 comprising or consisting of the amino acid sequence of SEQ ID NO: 1, or a functional homologue thereof with an amino acid sequence that is at least 80% identical to SEQ ID NO: 1.

In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is Bgall1 comprising or consisting of the amino acid sequence of SEQ ID NO: 2, or a functional homologue thereof with an amino acid sequence that is at least 80% identical to SEQ ID NO: 2.

In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is Bbac1 comprising or consisting of the amino acid sequence of SEQ ID NO: 3, or a functional homologue thereof with an amino acid sequence that is at least 80% identical to SEQ ID NO: 3.

In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is Murba1 comprising or consisting of the amino acid sequence of SEQ ID NO: 4, or a functional homologue thereof with an amino acid sequence that is at least 80% identical to SEQ ID NO: 4.

In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is Bacfin1 comprising or consisting of the amino acid sequence of SEQ ID NO: 5, or a functional homologue thereof with an amino acid sequence that is at least 80% identical to SEQ ID NO: 5

In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is Prev1 comprising or consisting of the amino acid sequence of SEQ ID NO: 6, or a functional homologue thereof with an amino acid sequence that is at least 80% identical to SEQ ID NO: 6-In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is Csec1 comprising or consisting of the amino acid sequence of SEQ ID NO: 7, or a functional homologue thereof with an amino acid sequence that is at least 80% identical to SEQ ID NO: 7. In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is CafC comprising or consisting of the amino acid sequence of SEQ ID NO: 8, or a functional homologue thereof with an amino acid sequence that is at least 80% identical to SEQ ID NO: 8.

In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is futA_mut2 comprising or consisting of the amino acid sequence of SEQ ID NO: 9, or a functional homologue thereof with an amino acid sequence that is at least 80% identical to SEQ ID NO: 9.

In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is FucT109 comprising or consisting of the amino acid sequence of SEQ ID NO: 10, or a functional homologue thereof with an amino acid sequence that is at least 80% identical to SEQ ID NO: 10.

In one embodiment the genetically engineered cell capable of producing LNDFH-III, comprises a recombinant nucleic acid sequence encoding an α-1,3-fucosyltransferase with dual α-1,3-fucosyltransferase activity, wherein the glycosyltransferase is FutA variants comprising substitutions at positions 128, 129 of SEQ ID NO: 11, wherein the variants have at least 80% identity, but less than 100% to SEQ ID NO: 11.

In one aspect of the invention, the genetically engineered cell comprises a recombinant nucleic acid sequence encoding an α-1,3-fucosyltransferase with α-1,3 (4)-fucosyltransferase activity, which is capable of producing at least 20 molar % LNDFH-III of the total molar HMO content produced by the cell. Preferably, the at least 25 molar % of the molar content of the total HMOs produced by said cell is LNDFH-III. In embodiments, at least at least 20 molar %, such as at least 25 molar %, 29 molar %, 30 molar %, 35 molar %, 40 molar %, 45 molar %, 50 molar %, 55 molar %, 60 molar %, or such as at least 65 molar % of the molar content of the total HMOs produced by said cell is LNDFH-III. In additional embodiments, the cell further produces one or more HMOs selected from the group consisting of 3FL, LNnT, LNFP-III and LNFP-VI.

In some embodiments, the genetically engineered cell described herein expresses Osc1 comprising or consisting of the amino acid sequence of SEQ ID NO: 1, or an amino acid sequence with at least 80%, such as at least 85%, such as at least 90%, such as at least 95%, or such as at least 99% sequence identity to SEQ ID NO: 1, and the molar % content of LNDFH-III produced by the genetically engineered cell is above 30 molar %, such as above 35 molar %, such as above 40 molar %, such as above 45 molar %, such as above 50 molar %, or such as above 55 molar % of the total HMO produced.

In some embodiments, the genetically engineered cell described herein expresses Bgall1 comprising or consisting of the amino acid sequence of SEQ ID NO: 2, or an amino acid sequence with at least 80%, such as at least 85%, such as at least 90%, such as at least 95%, or such as at least 99% sequence identity to SEQ ID NO: 2, and the molar % content of LNDFH-III produced by the genetically engineered cell is above 30 molar %, such as above 40 molar %, such as above 50 molar %, such as above 55 molar %, such as above 60 molar %, or such as above 65 molar % of the total HMO produced.

In some embodiments, the genetically engineered cell described herein expresses Bbac1 comprising or consisting of the amino acid sequence of SEQ ID NO: 3, or an amino acid sequence with at least 80%, such as at least 85%, such as at least 90%, such as at least 95%, or such as at least 99% sequence identity to SEQ ID NO: 3, and the molar % content of LNDFH-III produced by the genetically engineered cell is above 30 molar %, such as above 35 molar %, such as above 40 molar %, or such as above 45 molar % of the total HMO produced.

In some embodiments, the genetically engineered cell described herein expresses Murba1 comprising or consisting of the amino acid sequence of SEQ ID NO: 4, or an amino acid sequence with at least 80%, such as at least 85%, such as at least 90%, such as at least 95%, or such as at least 99% sequence identity to SEQ ID NO: 4, and the molar % content of LNDFH-III produced by the genetically engineered cell is above 30 molar %, such as above 35 molar %, such as above 40 molar %, or such as above 42 molar % of the total HMO produced.

In some embodiments, the genetically engineered cell described herein expresses Bacfin1 comprising or consisting of the amino acid sequence of SEQ ID NO: 5, or an amino acid sequence with at least 80%, such as at least 85%, such as at least 90%, such as at least 95%, or such as at least 99% sequence identity to SEQ ID NO: 5, and the molar % content of LNDFH-III produced by the genetically engineered cell is above 30 molar %, such as above 40 molar %, such as above 50 molar %, such as above 55 molar %, or such as above 60 molar % of the total HMO produced.

In some embodiments, the genetically engineered cell described herein expresses Prev1 comprising or consisting of the amino acid sequence of SEQ ID NO: 6, or an amino acid sequence with at least 80%, such as at least 85%, such as at least 90%, such as at least 95%, or such as at least 99% sequence identity to SEQ ID NO: 6, and the molar % content of LNDFH-III produced by the genetically engineered cell is above 25 molar %, such as above 30 molar %, such as above 35 molar %, or such as above 40 molar % of the total HMO produced.

In some embodiments, the genetically engineered cell described herein expresses Csec1 comprising or consisting of the amino acid sequence of SEQ ID NO: 7, or an amino acid sequence with at least 80%, such as at least 85%, such as at least 90%, such as at least 95%, or such as at least 99% sequence identity to SEQ ID NO: 7, and the molar % content of LNDFH-III produced by the genetically engineered cell is above 20 molar %, such as above 21 molar %, such as above 22 molar %, such as above 23 molar %, such as above 24 molar %, or such as above 25 molar % of the total HMO produced.

In some embodiments, the genetically engineered cell described herein expresses CafC comprising or consisting of the amino acid sequence of SEQ ID NO: 8, or an amino acid sequence with at least 80%, such as at least 85%, such as at least 90%, such as at least 95%, or such as at least 99% sequence identity to SEQ ID NO: 8, and the molar % content of LNDFH-III produced by the genetically engineered cell is above 24 molar %, such as above 25 molar %, such as above 26 molar %, such as above 27 molar %, such as above 28 molar %, or such as above 29 molar % of the total HMO produced.

In some embodiments, the genetically engineered cell described herein expresses a FutA variant comprising substitutions at positions 128, 129 of SEQ ID NO: 11, wherein the variant has at least 80% identity, but less than 100% to SEQ ID NO: 11, and the molar % content of LNDFH-III produced by the genetically engineered cell is above 25 molar %, such as above 30 molar %, such as above 35 molar %, or such as above 40 molar % of the total HMO produced.

In some embodiments, the genetically engineered cell described herein expresses FutA_mut2 comprising or consisting of the amino acid sequence of SEQ ID NO: 9, and the molar % content of LNDFH-III produced by the genetically engineered cell is above 25 molar %, such as above 30 molar %, such as above 35 molar %, or such as above 40 molar % of the total HMO produced.

In some embodiments, the genetically engineered cell described herein expresses FucT109 comprising or consisting of the amino acid sequence of SEQ ID NO: 10, or a functional homologue thereof with an amino acid sequence that is at least 80% identical to SEQ ID NO: 10, and the molar % content of LNDFH-III produced by the genetically engineered cell is above 15 molar %, such as above 17 molar %, such as above 19 molar %, such as above 21 molar %, or such as above 23 molar % of the total HMO produced.

More than 60% of the HMOs found in human milk are different species of fucosylated HMOs. Accordingly, in embodiments, at least 60%, such as at least 75%, 80%, 85%, 90%, 95% or at least 97% of the molar content of the total HMOs produced by said cell are fucosylated. Preferably, the HMOs produced by the cell are selected from the group consisting of 3FL, LNFP-III, LNFP-VI and LNDFH-III.

In some embodiments, the genetically engineered cell described herein expresses Osc1 comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1, and the molar % content of fucosylated HMOs produced by the genetically engineered cell is above 85 molar %, such as above 90 molar %, such as above 95 molar %, such as above 97 molar %, such as above 98 molar %, or such as above 99 molar % of the total HMO produced.

In some embodiments, the genetically engineered cell described herein expresses Osc1 comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1, only produces fucosylated HMOs.

In some embodiments, the genetically engineered cell described herein expresses Bgall1 comprising or consisting of the amino acid sequence of SEQ ID NO: 2, or an amino acid sequence with at least 80%, such as at least 85%, such as at least 90%, such as at least 95%, or such as at least 99% sequence identity to SEQ ID NO: 2, and the molar % content of fucosylated HMOs produced by the genetically engineered cell is above 85 molar %, such as above 90 molar %, such as above 95 molar %, such as above 97 molar %, such as above 98 molar %, or such as above 99 molar % of the total HMO produced.

In some embodiments, the genetically engineered cell described herein expresses Bgall1 comprising or consisting of the amino acid sequence of SEQ ID NO: 2, or an amino acid sequence with at least 80%, such as at least 85%, such as at least 90%, such as at least 95%, or such as at least 99% sequence identity to SEQ ID NO: 2, only produces fucosylated HMOs.

In some embodiments, the genetically engineered cell described herein expresses Bbac1 comprising or consisting of the amino acid sequence of SEQ ID NO: 3, or an amino acid sequence with at least 80%, such as at least 85%, such as at least 90%, such as at least 95%, or such as at least 99% sequence identity to SEQ ID NO: 3, and the molar % content of fucosylated HMOs produced by the genetically engineered cell is above 80 molar %, such as above 85 molar %, such as above 90 molar %, such as above 93 molar %, or such as above 95 molar % of the total HMO produced.

In some embodiments, the genetically engineered cell described herein expresses Murba1 comprising or consisting of the amino acid sequence of SEQ ID NO: 4, or an amino acid sequence with at least 80%, such as at least 85%, such as at least 90%, such as at least 95%, or such as at least 99% sequence identity to SEQ ID NO: 4, and the molar % content of fucosylated HMOs produced by the genetically engineered cell is above 70 molar %, such as above 75 molar %, such as above 80 molar %, such as above 82 molar %, or such as above 84 molar % of the total HMO produced.

In some embodiments, the genetically engineered cell described herein expresses Bacfin1 comprising or consisting of the amino acid sequence of SEQ ID NO: 5, or an amino acid sequence with at least 80%, such as at least 85%, such as at least 90%, such as at least 95%, or such as at least 99% sequence identity to SEQ ID NO: 5, and the molar % content of fucosylated HMOs produced by the genetically engineered cell is above 75 molar %, such as above 80 molar %, such as above 85 molar %, such as above 87 molar %, or such as above 89 molar % of the total HMO produced.

In some embodiments, the genetically engineered cell described herein expresses Prev1 comprising or consisting of the amino acid sequence of SEQ ID NO: 6, or an amino acid sequence with at least 80%, such as at least 85%, such as at least 90%, such as at least 95%, or such as at least 99% sequence identity to SEQ ID NO: 6, and the molar % content of fucosylated HMOs produced by the genetically engineered cell is above 75 molar %, such as above 80 molar %, such as above 85 molar %, such as above 87 molar %, or such as above 90 molar % of the total HMO produced.

In some embodiments, the genetically engineered cell described herein expresses Csec1 comprising or consisting of the amino acid sequence of SEQ ID NO: 7, or an amino acid sequence with at least 80%, such as at least 85%, such as at least 90%, such as at least 95%, or such as at least 99% sequence identity to SEQ ID NO: 7, and the molar % content of fucosylated HMOs produced by the genetically engineered cell is above 50 molar %, such as above 55 molar %, such as above 57 molar %, such as above 59 molar %, or such as above 61 molar % of the total HMO produced.

In some embodiments, the genetically engineered cell described herein expresses CafC comprising or consisting of the amino acid sequence of SEQ ID NO: 8, or an amino acid sequence with at least 80%, such as at least 85%, such as at least 90%, such as at least 95%, or such as at least 99% sequence identity to SEQ ID NO: 8, and the molar % content of fucosylated HMOs produced by the genetically engineered cell is above 85 molar %, such as above 90 molar %, such as above 95 molar %, such as above 97 molar %, such as above 98 molar %, or such as above 99 molar % of the total HMO produced.

In some embodiments, the genetically engineered cell described herein expresses a FutA variant comprising substitutions at positions 128, 129 of SEQ ID NO: 11, wherein the variant has at least 80% identity, but less than 100% to SEQ ID NO: 11, and the molar % content of fucosylated HMOs produced by the genetically engineered cell is above 80 molar %, such as above 85 molar %, such as above 90 molar %, such as above 93 molar %, such as above 95 molar %, or such as above 96 molar % of the total HMO produced.

In some embodiments, the genetically engineered cell described herein expresses FutA_mut2 comprising or consisting of the amino acid sequence of SEQ ID NO: 9, and the molar % content of fucosylated HMOs produced by the genetically engineered cell is above 80 molar %, such as above 85 molar %, such as above 90 molar %, such as above 93 molar %, such as above 95 molar %, or such as above 96 molar % of the total HMO produced.

In some embodiments, the genetically engineered cell described herein expresses FucT109 comprising or consisting of the amino acid sequence of SEQ ID NO: 10, or a functional homologue thereof with an amino acid sequence that is at least 80% identical to SEQ ID NO: 10, and the molar % content of fucosylated HMOs produced by the genetically engineered cell is above 60 molar %, such as above 63 molar %, such as above 65 molar %, such as above 67 molar %, or such as above 69 molar % of the total HMO produced.

In preferred embodiments, the fucosylated HMOs produced by a cell described herein are selected from LNDFH-II and the group consisting of 3FL, LNFP-III, LNFP-VI.

The genetically engineered cell described herein preferably expresses genes encoding key enzymes for the biosynthesis of fucosylated HMOs. In addition, it is advantageous if the genetically engineered cell expresses the genes needed to produce LNnT, either from lactose or LNT-II as the initial substrate (see FIG. 1), and/or alternatively the cell expresses importers for LNT-II or LNnT.

In embodiments the genetically engineered cell comprises one or more additional glycosyltransferases. The additional one or more glycosyltransferases are preferably selected from the group consisting of, galactosyltransferases, glucosaminyltransferases, fucosyltransferases and N-acetylglucosaminyl transferases.

In some embodiments the genetically engineered cell comprises one or more recombinant nucleic acid sequence(s) encoding a β-1,4-galactosyltransferase, and optionally a β-1,3-N-acetylglucosaminyltransferase. In some embodiments the β-1,3-N-acetylglucosaminyltransferase is from Neisseria meningitidis, and the β-1,4-galactosyltransferase is from Helicobacter pylori.

In some embodiments, a genetically engineered cell described herein further expresses the de novo GDP-fucose pathway genes responsible for the formation of GDP-fucose manA, manB, manC, gmd and wcaG. It may be advantageous to overexpress one or more of these genes and/or to upregulate the colanic acid gene cluster (CA), including the genes gmd, wcaG, wcaH, wcal, manC and manB from E. coli, through introduction of a nucleic acid construct encoding the CA as shown in SEQ ID NO: 23, allowing for formation of GDP-fucose, which enables the cell to produce a higher level of fucosylated oligosaccharides from one or more intermediate oligosaccharide substrates, such as lactose or LNnT, LNFP-III and/or LNFP-VI. Depending on the intended use of substrate, one or more additional glycosyltransferases and pathways for producing nucleotide-activated sugars, such as glucose-UDP-GlcNAc, CMP-N-acetylneuraminic acid, UDP-galactose, UDP-glucose, UDP-N-acetylglucosamine, UDP-N-acetylgalactosamine and/or CMP-N-acetylneuraminic acid can also be present in the genetically engineered cell.

It is further understood that the genetically engineered cell described herein may further comprise any of the modifications described above, e.g., additional glycosyltransferases, suitable importer proteins such as overexpression of lactose permease, LNT-II or LNT importers, beta-galactosidase inactivation in particular if lactose is used as the initial substrate, as well suitable exporter proteins for the complex fucosylated HMOs produced by the cell.

### HMO Mixtures Produced by the Cell

The genetically engineered cell comprising an α-1,3-fucosyltransferase described herein with dual fucosyltransferase specificity will generally produce a mixture of HMOs as a result of the multistep process inside the cell towards the final HMO product, LNDFH-III (see FIG. 1). In the production of LNDFH-III from lactose as the initial substrate, it is expected that certain amounts of 3FL (fucosylated lactose), LNT-II, LNnT, LNFP-III and LNFP-VI will be produced by the cell, with some species only being produced as synthesis intermediates, that are not present in the final mixture produced by the cell.

The molar % of individual HMO components supported by experimental data from the Examples shows exemplary HMO composition ranges, wherein the mixture of final HMOs products consists essentially of LNDFH-III and one or more HMOs selected from the group consisting of 3-FL, LNT-II, LNT, LNFP-III and LNFP-VI. Preferably, LNT-II is in very low amounts or is not present in detectable amounts.

In embodiments, the cell produces a final mixture consisting essentially of LNDFH-III and 3FL. In another embodiment, the cell produces a final mixture consisting essentially of LNDFH-III, LNFP-III and 3FL. In another embodiment, the cell produces a final mixture consisting essentially of LNDFH-III, LNFP-VI and 3FL. In another embodiment, the cell produces a final mixture consisting essentially of LNDFH-III, LNFP-III, LNFP-VI and 3FL. In another embodiment, the cell produces a final mixture consisting essentially of LNDFH-III, LNFP-III, 3FL and LNnT. In another embodiment, the cell produces a final mixture consisting essentially of LNDFH-III, LNFP-III, LNFP-VI, 3FL and LNnT.

In embodiments, the cell produces a mixture of HMOs consisting essentially of 20-70 molar % of LNDFH-III, 0-35 molar % LNFP-III, 0-35 molar % LNDFP-VI, 0-65 molar % 3FL, 0-40% LNnT, and at the most 1% pLNnH, in total adding up to 100% molar content.

In some embodiments, the genetically engineered cell described herein expresses Osc1 comprising or consisting of the amino acid sequence of SEQ ID NO: 1, or an amino acid sequence with at least 80%, such as at least 85%, such as at least 90%, such as at least 95%, or such as at least 99% sequence identity to SEQ ID NO: 1, and the produced mixture consists essentially of 30-60 molar % of LNDFH-III and 40-65 molar % 3FL, in total adding up to 100% molar content.

In some embodiments, the genetically engineered cell described herein expresses Bgall1 comprising or consisting of the amino acid sequence of SEQ ID NO: 2, or an amino acid sequence with at least 80%, such as at least 85%, such as at least 90%, such as at least 95%, or such as at least 99% sequence identity to SEQ ID NO: 2, and the produced mixture consists essentially of 50-80 molar % LNDFH-III, 1-10 molar % LNFP-III and 15-50% 3FL, in total adding up to 100% molar content.

In some embodiments, the genetically engineered cell described herein expresses Bbac1 comprising or consisting of the amino acid sequence of SEQ ID NO: 3, or an amino acid sequence with at least 80%, such as at least 85%, such as at least 90%, such as at least 95%, or such as at least 99% sequence identity to SEQ ID NO: 3, and the produced mixture consists essentially of 40-50 molar % LNDFH-III, 5-15 molar % LNFP-VI, 3-8 molar % LNFP-III and 2-12 molar % LNnT and 30-40 molar % 3FL, in total adding up to 100% molar content.

In some embodiments, the genetically engineered cell described herein expresses Murba1 comprising or consisting of the amino acid sequence of SEQ ID NO: 4, or an amino acid sequence with at least 80%, such as at least 85%, such as at least 90%, such as at least 95%, or such as at least 99% sequence identity to SEQ ID NO: 4, and the produced mixture consists essentially of 40-50 molar % LNDFH-III, 25-35 molar % LNFP-VI, 10-20 molar % LNnT and 5-15 molar % 3FL, in total adding up to 100% molar content.

In some embodiments, the genetically engineered cell described herein expresses Bacfin1 comprising or consisting of the amino acid sequence of SEQ ID NO: 5, or an amino acid sequence with at least 80%, such as at least 85%, such as at least 90%, such as at least 95%, or such as at least 99% sequence identity to SEQ ID NO: 5, and the produced mixture consists essentially of 55-65 molar % LNDFH-III, 17-27 molar % LNFP-III and 6-16 molar % LNnT and 1-11 molar % 3FL, in total adding up to 100% molar content.

In some embodiments, the genetically engineered cell described herein expresses Prev1 comprising or consisting of the amino acid sequence of SEQ ID NO: 6, or an amino acid sequence with at least 80%, such as at least 85%, such as at least 90%, such as at least 95%, or such as at least 99% sequence identity to SEQ ID NO: 6, and the produced mixture consists essentially of 36-46 molar % LNDFH-III, 25-35 molar % LNFP-VI, 10-20 molar % LNFP-III and 2-12 molar % LNnT and 2-12 molar % 3FL, in total adding up to 100% molar content.

In some embodiments, the genetically engineered cell described herein expresses Csec1 comprising or consisting of the amino acid sequence of SEQ ID NO: 7, or an amino acid sequence with at least 80%, such as at least 85%, such as at least 90%, such as at least 95%, or such as at least 99% sequence identity to SEQ ID NO: 7, and the produced mixture consists essentially of 25-35 molar % LNDFH-III, 15-25 molar % LNFP-VI, 8-18 molar % LNFP-III and 33-43 molar % LNnT, in total adding up to 100% molar content.

In some embodiments, the genetically engineered cell described herein expresses CafC comprising or consisting of the amino acid sequence of SEQ ID NO: 8, or an amino acid sequence with at least 80%, such as at least 85%, such as at least 90%, such as at least 95%, or such as at least 99% sequence identity to SEQ ID NO: 8, and the produced mixture consists essentially of 25-35 molar % LNDFH-III, 29-39 molar % LNFP-VI and 31-41% 3FL, in total adding up to 100% molar content.

In some embodiments, the genetically engineered cell described herein expresses FutA_mut2 comprising or consisting of the amino acid sequence of SEQ ID NO: 9, and the produced mixture consists essentially of 37-47 molar % LNDFH-III, 26-36 molar % LNFP-III, 19-29 molar % 3FL, and less than 5% LNnT, in total adding up to 100% molar content.

In some embodiments, the genetically engineered cell described herein expresses FucT109 comprising or consisting of the amino acid sequence of SEQ ID NO: 10, or an amino acid sequence with at least 80%, such as at least 85%, such as at least 90%, such as at least 95%, or such as at least 99% sequence identity to SEQ ID NO: 10, and the produced mixture consists essentially of 19-29 molar % LNDFH-III, 13-23 molar % LNFP-VI, 22-32 molar % LNFP-III and 24-34 molar % LNnT and less than 5 molar % 3FL, in total adding up to 100% molar content.

In some embodiments, the genetically engineered cell described herein expresses a FutA variant comprising substitutions at positions 128, 129 of SEQ ID NO: 11, wherein the variant has at least 80% identity, but less than 100% to SEQ ID NO: 11, and the produced mixture consists essentially of 37-47 molar % LNDFH-III, 26-36 molar % LNFP-III, 19-29 molar % 3FL, and less than 5% LNnT, in total adding up to 100% molar content.

### Host Cells

In embodiments, the engineered cell is a microorganism. The genetically engineered cell is preferably a microbial cell, such as a prokaryotic cell or eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be member of the Enterobacterales order, preferably of the genus Escherichia, more preferably of the species E. coli. Other examples of suitable host cell are Erwinia herbicola (Pantoea agglomerans), Citrobacter freundii, Campylobacter sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species. Also included as useful species are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a heterologous product are e.g., yeast cells, such as Komagataella, Kluyveromyces, Yarrowia, Pichia, Saccaromyces, Schizosaccharomyces or Hansenula or from a filamentous fungus of the genera Aspargillus, Fusarium or Thricoderma.

In one or more exemplary embodiments, the genetically engineered cell is selected from the group consisting of Escherichia sp., Bacillus sp., Lactobacillus sp., Corynebacterium sp. and Campylobacter sp.

In one or more exemplary embodiments, the genetically engineered cell is selected from the group consisting of Escherichia coli, Bacillus subtilis, Lactobacillus lactis, Corynebacterium glutamicum, Yarrowia lipolytica, Pichia pastoris, and Saccharomyces cerevisiae.

In one or more exemplary embodiments, the genetically engineered cell is B. subtilis.

In one or more exemplary embodiments, the genetically engineered cell is S. Cerevisiae or P pastoris.

In one or more exemplary embodiments, the genetically engineered cell is Escherichia coli.

In one or more exemplary embodiments, the invention relates to a genetically engineered cell, wherein the cell is derived from the E. coli K-12 strain or DE3.

### a Recombinant Nucleic Acid Sequence

The present invention relates to a genetically engineered cell comprising a recombinant nucleic acid sequence encoding an α-1,3-fucosyltransferase with dual α-1,3-fucosyltransferase activity, such as an enzyme selected from the group consisting of Osc1, Bgall1, Bbac1, Murba1, Bacfin1, Prev1, Csec1, CafC, FucT109 and FutA_mut2, wherein said cell produces Human Milk Oligosaccharides (HMO). In particular, at least one fucosylated HMO, and preferably with a molar % content of LNDFH-III above 25%, such as above 50% of the total HMO produced.

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e., a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence or other non-coding regulatory sequences.

The recombinant nucleic acid sequence may in addition be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

The invention also relates to a nucleic acid construct comprising a coding nucleic sequence, i.e. recombinant DNA sequence of a gene of interest, e.g., an α-1,3-fucosyltransferase gene, and a non-coding regulatory DNA sequence, e.g., a promoter DNA sequence, e.g., a recombinant promoter sequence derived from the promoter sequence of the lac operon or the glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. It refers to the functional relationship of a transcriptional regulatory sequence to a transcribed sequence. E.g., a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the invention may be a part of the vector DNA, in another embodiment, the construct it is an expression cassette/cartridge that is integrated in the genome of a host cell.

Accordingly, the term “nucleic acid construct” means an artificially constructed segment of nucleic acids, in particular a DNA segment, which is intended to be inserted into a target cell, e.g., a bacterial cell, to modify expression of a gene of the genome or expression of a gene/coding DNA sequence which may be included in the construct. Thus, in embodiments, the present invention relates to a nucleic acid construct comprising a recombinant nucleic acid sequence encoding an α-1,3-fucosyltransferase, wherein said recombinant nucleic acid sequence is selected from the group consisting of nucleic acid sequences encoding Osc1, Bgall1, Bbac1, Murba1, Bacfin1, Prev1, Csec1, CafC, FutA_mut2, FucT109, such as a nucleic acid sequence according to SEQ ID NO: 12, 13, 14, 15, 16, 17, 18, 19, 20, or 21, or functional variants thereof.

The genetically engineered cell according to the present invention may also comprise multiple copies of the recombinant nucleic acid sequence encoding an α-1,3-fucosyltransferase. Enhancing the copy number of the α-1,3-fucosyltransferase was shown in Example 1 to change the ratio of the produced HMOs. In specific it was shown that increasing the copy number of Bgall1 by introduction of two genomic copies resulted in an increase in 3FL production and a substantial reduction in LNDFH-III production.

Accordingly, the copy number variation may be used in the production to tailor specific HMOs mixtures, in this case a mixture comprising 3FL, LNFP-III, LNFP-VI and/or LNDFH-III in different ratios, depending on the need for the specific product.

Accordingly, in embodiments, the genetically engineered cell described herein comprises one, two, three or more genomic copies of the recombinant nucleic acid sequence encoding the glycosyltransferase selected from the group consisting of Osc1, Bgall1, Bbac1, Murba1, Bacfin1, Prev1, Csec1, CafC, FutA_mut2, FucT109, comprising or consisting of an amino acid sequence according to SEQ ID NO: 1, 2, 3, 4, 5, 6, 7, 8, 9 or 10, or a functional homologue thereof with an amino acid sequence that is at least 80% identical to SEQ ID NO: 1, 2, 3, 4, 5, 6, 7, 8, 9 or 10.

In one embodiment the genetically engineered cell comprises two, three or more genomic copies and/or a plasmid-borne copy of the recombinant nucleic acid sequence encoding a FutA variant comprising substitutions at positions 128, 129 of SEQ ID NO: 11, wherein the variants has at least 80% identity, but less than 100% to SEQ ID NO: 11.

In additional embodiments, the plasmid is a high copy number plasmid, preferably, a pUC57 or pBB-B9 plasmid.

One embodiment of the invention relates to a nucleic acid construct comprising a recombinant nucleic acid sequence encoding an α-1,3-fucosyltransferase, wherein said recombinant nucleic acid sequence is selected from the group consisting of a) Osc1 comprising or consisting of the nucleic acid sequences of SEQ ID NO: 12 or an nucleic acid sequence with at least 80%, such as at least 85%, such as at least 90% such as at least 95%, or such as at least 99% sequence identity to SEQ ID NO: 12; b) Bgall1 comprising or consisting of the nucleic acid sequences of SEQ ID NO: 13 or a nucleic acid sequence with at least 80%, such as at least 85%, such as at least 90% such as at least 95%, or such as at least 99% sequence identity to SEQ ID NO: 13; c) Bbac1 comprising or consisting of the nucleic acid sequences of SEQ ID NO: 14 or a nucleic acid sequence with at least 80%, such as at least 85%, such as at least 90% such as at least 95%, or such as at least 99% sequence identity to SEQ ID NO: 14; d) Murba1 comprising or consisting of the nucleic acid sequences of SEQ ID NO: 15 or a nucleic acid sequence with at least 80%, such as at least 85%, such as at least 90% such as at least 95%, or such as at least 99% sequence identity to SEQ ID NO: 15; e) Bacfin1 comprising or consisting of the nucleic acid sequences of SEQ ID NO: 16 or a nucleic acid sequence with at least 80%, such as at least 85%, such as at least 90% such as at least 95%, or such as at least 99% sequence identity to SEQ ID NO: 16; f) Prev1 comprising or consisting of the nucleic acid sequences of SEQ ID NO: 17 or a nucleic acid sequence with at least 80%, such as at least 85%, such as at least 90% such as at least 95%, or such as at least 99% sequence identity to SEQ ID NO: 17; g) Csec1 comprising or consisting of the nucleic acid sequences of SEQ ID NO: 18 or a nucleic acid sequence with at least 80%, such as at least 85%, such as at least 90% such as at least 95%, or such as at least 99% sequence identity to SEQ ID NO: 18; h) CafC comprising or consisting of the nucleic acid sequences of SEQ ID NO: 19 or a nucleic acid sequence with at least 80%, such as at least 85%, such as at least 90% such as at least 95%, or such as at least 99% sequence identity to SEQ ID NO: 19; i) FutA_mut2 comprising or consisting of the nucleic acid sequences of SEQ ID NO: 20 or a nucleic acid sequence with at least 80%, such as at least 85%, such as at least 90% such as at least 95%, or such as at least 99% sequence identity to SEQ ID NO: 20; and/or j) FucT109 comprising or consisting of the nucleic acid sequences of SEQ ID NO: 21 or a nucleic acid sequence with at least 80%, such as at least 85%, such as at least 90% such as at least 95%, or such as at least 99% sequence identity to SEQ ID NO: 21.

Preferably, the α-1,3-fucosyltransferase encoding sequence is under the control of a promoter sequence selected from promotor sequences with a nucleic acid sequence as identified in Table 4.

TABLE 4
Selected promoter sequences

| | % Activity | | | |
|---|---|---|---|---|
| | relative | | | Seq ID |
| Promoter name | to PglpF* | Strength | Reference | in appl. |
| PmglB_70UTR_SD8 | 291% | high | WO2020255054 | 27 |
| PmglB_70UTR_SD10 | 233-281% | high | WO2020255054 | 28 |
| PmglB_54UTR | 197% | high | WO2020255054 | 29 |
| Plac_70UTR | 182-220% | high | WO2019123324 | 30 |
| PmglB_70UTR_SD9 | 180-226% | high | WO2020255054 | 31 |
| PmglB_70UTR_SD4 | 153%-353% | high | WO2020255054 | 32 |
| PmglB_70UTR_SD5 | 146-152% | high | WO2020255054 | 33 |
| PglpF_SD4 | 140-161% | high | WO2019123324 | 34 |
| PmglB_70UTR_SD7 | 127-173% | high | WO2019123324 | 35 |
| PmglB_70UTR | 124-234% | high | WO2020255054 | 36 |
| PglpA_70UTR | 102-179% | high | WO2019123324 | 37 |
| PglpT_70UTR | 102-240% | high | WO2019123324 | 38 |
| PglpF | 100% | high | WO2019123324 | 39 |
| PglpF_SD10 | 88-96% | high | WO2019123324 | 40 |
| PglpF_SD5 | 82-91% | high | WO2019123324 | 41 |
| PglpF_SD8 | 81-82% | high | WO2019123324 | 42 |
| PmglB_16UTR | 78-171% | high | WO2019123324 | 43 |
| PglpF_SD9 | 73-93% | middle | WO2019123324 | 44 |
| PglpF_SD7 | 47-57% | middle | WO2019123324 | 45 |
| PglpF_SD6 | 46-47% | middle | WO2019123324 | 46 |
| PglpA_16UTR | 38-64% | middle | WO2019123324 | 47 |
| Plac_wt* | 15-28% | low | WO2019123324 | 48 |
| PglpF_SD3 | 9% | low | WO2019123324 | 49 |
| PglpF_SD1 | 5% | low | WO2019123324 | 50 |

*The promoter activity is assessed in the LacZ assay described below with the PglpF promoter run as positive reference in the same assay. To compare across assays the activity is calculated relative to the PglpF promoter, a range indicates results from multiple assays.

The promoter may be of heterologous origin, native to the genetically engineered cell or it may be a recombinant promoter, combining heterologous and/or native elements.

One way to increase the production of a product may be to regulate the production of the desired enzyme activity used to produce the product, such as the glycosyltransferases or enzymes involved in the biosynthetic pathway of the glycosyl donor.

Increasing the promoter strength driving the expression of the desired enzyme may be one way of doing this. The strength of a promoter can be assessed using a lacZ enzyme assay where β-galactosidase activity is assayed as described previously (see e.g., Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU. IN preferred embodiments, the expression of said nucleic acid sequences are under control of a strong promoter selected from the group consisting of SEQ ID NOs 27, 28, 29, 30, 31, 32, 33, 34, 35, 36, 37 and 38.

In embodiments the expression of said nucleic acid sequences described herein is under control of a PglpF (SEQ ID NO: 39) or Plac (SEQ ID NO: 48) promoter or PmglB_UTR70 (SEQ ID NO: 36) or PglpA_70UTR (SEQ ID NO: 37) or PglpT_70UTR (SEQ ID NO: 38) or variants thereof such as promoters identified in Table 4, in particular the PglpF_SD4 variant of SEQ ID NO: 34 or Plac_70UTR variant of SEQ ID NO: 30, or PmglB_70UTR variants of SEQ ID NO: 27, 28, 29, 31, 32, 33, 35 and 36. Further suitable variants of PglpF, PglpA_70UTR, PglpT_70UTR and PmglB_70UTR promoter sequences are described in or WO2019/123324 and WO2020/255054 respectively (hereby incorporated by reference).

In preferred embodiments, the recombinant nucleic acid sequences individually are under the control of one or more promoters selected from the group consisting of PglpF, Plac, PmglB_70UTR, PglpA_70UTR and PglpT_70UTR (SEQ ID NOs: 39, 48, 36, 37 and 38, respectively) and variants thereof.

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2 (2): 137-49.); methods for genomic integration of nucleic acid sequences in which recombination is mediated by the Red recombinase function of the phage λ or the RecE/RecT recombinase function of the Rac prophage (Murphy, J Bacteriol. (1998); 180 (8): 2063-7; Zhang et al., Nature Genetics (1998) 20:123-128 Muyrers et al., EMBO Rep. (2000) 1 (3): 239-243); methods based on Red/ET recombination (Wenzel et al., Chem Biol. (2005), 12 (3): 349-56.; Vetcher et al., Appl Environ Microbiol. (2005); 71 (4): 1829-35); or positive clones, i.e., clones that carry the expression cassette, can be selected e.g., by means of a marker gene, or loss or gain of gene function.

In one or more exemplary embodiments, the present disclosure relates to one or more recombinant nucleic acid sequences as illustrated in SEQ ID NOs: 12, 13, 14, 15, 16, 17, 18, 19, 20 and 21 [nucleic acid sequence encoding Osc1, Bgall1, Bbac1, Murba1, Bacfin1, Prev1, Csec1, CafC, FutA_mut2 and FucT109].

In particular, the present disclosure relates to one or more of a recombinant nucleic acid sequence and/or to a functional homologue thereof having a sequence which is at least 70% identical to SEQ ID NOs: 12, 13, 14, 15, 16, 17, 18, 19, 20 or 21 [nucleic acid encoding Osc1, Bgall1, Bbac1, Murba1, Bacfin1, Prev1, Csec1, CafC, FutA_mut2 and FucT109 respectivley], such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence of the invention) and a reference sequence (such as a prior art sequence) based on their pairwise alignment. For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “identity” (obtained using the -nobrief option) is used as the percent identity. Generally sequence identity may be calculated as follows: (Identical Residues×100)/(Length of Aligned region).

For purposes of the present invention, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16:276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “identity” (obtained using the -nobrief option) is used as the percent identity. Generally sequence identity may be calculated as follows: (Identical Deoxyribonucleotides×100)/(Length of Aligned region).

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences shown in table 1 or a recombinant nucleic acid encoding any one of the sequences of SEQ ID NO: 12, 13, 14, 15, 16, 17, 18, 19, 20 and 21, should ideally be able to participate in the production of fucosylated HMOs, in terms of increased HMO yield, export of HMO product out of the cell or import of substrate for the HMO production, such as a acceptor oligosaccharide of at least three monosaccharide units, improved purity/by-product formation, reduction in biomass formation, viability of the genetically engineered cell, robustness of the genetically engineered cell according to the disclosure, or reduction in consumables needed for the production.

### Use of a Genetically Engineered Cell or Enzyme

The disclosure also relates to any commercial use of the enzyme(s), genetically engineered cell(s) or the nucleic acid construct(s) disclosed herein, such as, but not limited to, in a method for producing one or more fucosylated human milk oligosaccharide (HMO), preferably, LNDFH-III.

Accordingly, the present disclosure also relates to the use of an α-1,3-fucosyltransferase in production of a fucosylated product comprising LNDFH-III, wherein the α-1,3-fucosyltransferase is selected from the group consisting of Osc1, Bgall1, Bbac1, Murba1, Bacfin1, Prev1, Csec1, CafC, FutA_mut2 and FucT109 comprising or consisting of the amino acid sequence of SEQ ID NO: 1, 2, 3, 4, 5, 6, 7, 8, 9 or 10, or a functional homologue thereof with an amino acid sequence that is at least 80% identical to SEQ ID NO: 1, 2, 3, 4, 5, 6, 7, 8, 9 or 10. In further embodiments, the α-1,3-fucosyltransferase for use in production of a fucosylated product is a FutA variant comprising substitutions at positions 128, 129 of SEQ ID NO: 11, wherein the variant has at least 80% identity, but less than 100% to SEQ ID NO: 11.

In further embodiments, the α-1,3-fucosyltransferase for use in production of a fucosylated product is selected from the group consisting of a) Osc1 comprising or consisting of the amino acid sequence of SEQ ID NO: 1, or a functional homologue thereof with an amino acid sequence that is at least 80% identical to SEQ ID NO: 1, b) Bgall1 comprising or consisting of the amino acid sequence of SEQ ID NO: 2, or a functional homologue thereof with an amino acid sequence that is at least 80% identical to SEQ ID NO: 2, c) Bbac1 comprising or consisting of the amino acid sequence of SEQ ID NO: 3, or a functional homologue thereof with an amino acid sequence that is at least 80% identical to SEQ ID NO: 3, wherein the fucosylated product comprises LNDFH-III with low levels of LNFP-III and LNFP-VI, such as less than 10% of the total HMO of each. Preferably the α-1,3-fucosyltransferases produce HMO mixtures LNDFH-III and 3FL, where these two HMOs constitute at least 80%, such as at least 90% of the total HMO produced.

In further embodiments, the α-1,3-fucosyltransferase for use in production of a fucosylated product is selected from the group consisting of a) Bgall1 comprising or consisting of the amino acid sequence of SEQ ID NO: 2, or a functional homologue thereof with an amino acid sequence that is at least 80% identical to SEQ ID NO: 2, b) Bacfin1 comprising or consisting of the amino acid sequence of SEQ ID NO: 5, or a functional homologue thereof with an amino acid sequence that is at least 80% identical to SEQ ID NO: 5, c) Murba1 comprising or consisting of the amino acid sequence of SEQ ID NO: 4, or a functional homologue thereof with an amino acid sequence that is at least 80% identical to SEQ ID NO: 4 and d) FutA variants comprising substitutions at a position corresponding to position 128 and 129 of SEQ ID NO: 11, wherein the variants have at least 80% sequence identity to SEQ ID NO: 11, where the fucosylated product comprises LNFP-III, and LNDFH-III, with LNFP-VI constituting less the less than 1% of the total HMO produced.

In further embodiments, the α-1,3-fucosyltransferase for use in production of a fucosylated product is selected from the group consisting of a) Bbac1 comprising or consisting of the amino acid sequence of SEQ ID NO: 3, or a functional homologue thereof with an amino acid sequence that is at least 80% identical to SEQ ID NO: 3, b) Prev1 comprising or consisting of the amino acid sequence of SEQ ID NO: 6, or a functional homologue thereof with an amino acid sequence that is at least 80% identical to SEQ ID NO: 6, c) FucT109 comprising or consisting of the amino acid sequence of SEQ ID NO: 10, or a functional homologue thereof with an amino acid sequence that is at least 80% identical to SEQ ID NO: 10, d) Csec1 comprising or consisting of the amino acid sequence of SEQ ID NO: 7, or a functional homologue thereof with an amino acid sequence that is at least 80% identical to SEQ ID NO: 7, where the fucosylated product comprises LNDFH-III and LNFP-III and LNFP-VI.

In embodiments, the α-1,3-fucosyltransferases described herein are also used in the manufacturing of a fucosylated product, wherein the fucosylated product comprises one or more fucosylated oligosaccharides including LNDFH-III.

In an exemplified embodiment, the genetically engineered cell and/or the nucleic acid construct described herein is used in the manufacturing of HMOs. Preferably, in the manufacturing of mixtures of HMOs, wherein the molar % content of LNDFH-III produced by the genetically engineered cell is above 20% of the total amount of HMO produced.

Preferably, in the manufacturing of HMOs, wherein a mixture of fucosylated HMOs comprising LNDFH-III is intended as the primary product, the molar % content of LNDFH-III produced by the genetically engineered cell is above 20% such as above 25%, such as above 30%, such as above 35%, such as above 40%, such as above 45%, such as above 50%, such as above 55%, such as above 60%, or such as above 65%, of the total amount of HMO produced.

In embodiments, the α-1,3-fucosyltransferase described herein are also used in the manufacturing of a fucosylated product, wherein the fucosylated product is one or more fucosylated oligosaccharides, such as one or more HMOs, preferably, a mixture of HMOs wherein at least 60% of the mixture consists of LNDFH-III in combination with LNFP-III, LNFP-VI and/or 3FL.

In an exemplified embodiment, the genetically engineered cell and/or the nucleic acid construct according to the invention is used in the manufacturing of one or more fucosylated HMO(s), preferably, LNDFH-III.

Production of these HMO's may require the presence of two or more glycosyltransferase activities.

### A Method for Producing Fucosylated Human Milk Oligosaccharides (HMOs)

The present invention also relates to a method for producing one or more fucosylated human milk oligosaccharide (HMO), preferably LNDFH-III, said method comprises culturing a genetically engineered cell according to the present invention.

The present disclosure relates to a method for producing one or more fucosylated human milk oligosaccharides (HMOs), said method comprising cultivating a genetically engineered cell, said cell comprising:
a recombinant nucleic acid sequence encoding an α-1,3-fucosyltransferase, wherein said fucosyltransferase is selected from the group consisting of:
   a. Osc1 comprising or consisting of the amino acid sequence of SEQ ID NO: 1, or a functional homologue thereof with an amino acid sequence that is at least 80% identical to SEQ ID NO: 1,
   b. Bgall1 comprising or consisting of the amino acid sequence of SEQ ID NO: 2, or a functional homologue thereof with an amino acid sequence that is at least 80% identical to SEQ ID NO: 2,
   c. Bbac1 comprising or consisting of the amino acid sequence of SEQ ID NO: 3, or a functional homologue thereof with an amino acid sequence that is at least 80% identical to SEQ ID NO: 3,
   d. Murba1 comprising or consisting of the amino acid sequence of SEQ ID NO: 4, or a functional homologue thereof with an amino acid sequence that is at least 80% identical to SEQ ID NO: 4,
   e. Bacfin1 comprising or consisting of the amino acid sequence of SEQ ID NO: 5, or a functional homologue thereof with an amino acid sequence that is at least 80% identical to SEQ ID NO: 5,
   f. Prev1 comprising or consisting of the amino acid sequence of SEQ ID NO: 6, or a functional homologue thereof with an amino acid sequence that is at least 80% identical to SEQ ID NO: 6, and
   g. Csec1 comprising or consisting of the amino acid sequence of SEQ ID NO: 7, or a functional homologue thereof with an amino acid sequence that is at least 80% identical to SEQ ID NO: 7.

In embodiments the genetically engineered cell is cultured in a suitable medium providing a suitable carbon source and in the presence of an initial substrate selected from lactose or LNT-II. Preferably, the initial substrate is lactose. A further embodiment is a method for producing one or more fucosylated human milk oligosaccharides (HMOs), said method comprising cultivating a genetically engineered cell comprising
a. a recombinant nucleic acid sequence encoding an α-1,3-fucosyltransferase, wherein said enzyme is selected from the group consisting of:
   i. Osc1 comprising or consisting of the amino acid sequence of SEQ ID NO: 1, or a functional homologue thereof with an amino acid sequence that is at least 80% identical to SEQ ID NO: 1,
   ii. Bgall1 comprising or consisting of the amino acid sequence of SEQ ID NO: 2, or a functional homologue thereof with an amino acid sequence that is at least 80% identical to SEQ ID NO: 2,
   iii. Bbac1 comprising or consisting of the amino acid sequence of SEQ ID NO: 3, or a functional homologue thereof with an amino acid sequence that is at least 80% identical to SEQ ID NO: 3,
   iv. Murba1 comprising or consisting of the amino acid sequence of SEQ ID NO: 4, or a functional homologue thereof with an amino acid sequence that is at least 80% identical to SEQ ID NO: 4,
   v. Bacfin1 comprising or consisting of the amino acid sequence of SEQ ID NO: 5, or a functional homologue thereof with an amino acid sequence that is at least 80% identical to SEQ ID NO: 5,
   vi. Prev1 comprising or consisting of the amino acid sequence of SEQ ID NO: 6, or a functional homologue thereof with an amino acid sequence that is at least 80% identical to SEQ ID NO: 6,
   vii. Csec1 comprising or consisting of the amino acid sequence of SEQ ID NO: 7, or a functional homologue thereof with an amino acid sequence that is at least 80% identical to SEQ ID NO: 7,
   viii. CafC comprising or consisting of the amino acid sequence of SEQ ID NO: 8, or a functional homologue thereof with an amino acid sequence that is at least 80% identical to SEQ ID NO: 8,
   ix. Fut_mut2 comprising or consisting of the amino acid sequence of SEQ ID NO: 9,
   x. FucT109 comprising or consisting of the amino acid sequence of SEQ ID NO: 10, or a functional homologue thereof with an amino acid sequence that is at least 80% identical to SEQ ID NO: 10 and
   xi. FutA variants comprising substitutions at a position corresponding to position 128 and 129 of SEQ ID NO: 11, wherein the variants have at least 80% sequence identity to SEQ ID NO: 11, and
b. a recombinant nucleic acid sequence encoding an enzyme with a β-1,4-galactosyltransferase activity, and
c. optionally, a recombinant nucleic acid sequence encoding an enzyme with β-1,3-N-acetyl-glucosaminyltransferase activity, and
d. cultivating said cell in a suitable medium in the presence of an initial substrate, and wherein at least one of the fucosylated HMOs is LNDFH-III. The fucosylated HMOs produced by the above method may further comprise a fucosylated HMO selected from group consisting of 3FL, LNFP-III and LNFP-VI and potentially also a non-fucosylated HMO such as LNnT.

A further embodiment relates to a method for producing one or more fucosylated human milk oligosaccharides (HMO), said method comprising cultivating a genetically engineered cell comprising
a. a recombinant nucleic acid sequence encoding an α-1,3-fucosyltransferase, wherein said enzyme is selected from the group consisting of:
   i) Osc1 comprising or consisting of the amino acid sequence of SEQ ID NO: 1, or a functional homologue thereof with an amino acid sequence that is at least 80% identical to SEQ ID NO: 1,
   ii) Bgall1 comprising or consisting of the amino acid sequence of SEQ ID NO: 2, or a functional homologue thereof with an amino acid sequence that is at least 80% identical to SEQ ID NO: 2,
   iii) Bbac1 comprising or consisting of the amino acid sequence of SEQ ID NO: 3, or a functional homologue thereof with an amino acid sequence that is at least 80% identical to SEQ ID NO: 3,
   iv) Murba1 comprising or consisting of the amino acid sequence of SEQ ID NO: 4, or a functional homologue thereof with an amino acid sequence that is at least 80% identical to SEQ ID NO: 4,
   v) Bacfin1 comprising or consisting of the amino acid sequence of SEQ ID NO: 5, or a functional homologue thereof with an amino acid sequence that is at least 80% identical to SEQ ID NO: 5,
   vi) Prev1 comprising or consisting of the amino acid sequence of SEQ ID NO: 6, or a functional homologue thereof with an amino acid sequence that is at least 80% identical to SEQ ID NO: 6 and
   vii) Csec1 comprising or consisting of the amino acid sequence of SEQ ID NO: 7, or a functional homologue thereof with an amino acid sequence that is at least 80% identical to SEQ ID NO: 7,
   viii) CafC comprising or consisting of the amino acid sequence of SEQ ID NO: 8, or a functional homologue thereof with an amino acid sequence that is at least 80% identical to SEQ ID NO: 8,
   ix) FutA_mut2 comprising or consisting of the amino acid sequence of SEQ ID NO: 9, and
b. a recombinant nucleic acid sequence encoding an enzyme with a β-1,4-galactosyltransferase activity;
c. optionally a recombinant nucleic acid sequence encoding an enzyme with β-1,3-N-acetyl-glucosaminyltransferase activity; and
d. cultivating said cell in a suitable medium in the presence of an initial substrate, and
   wherein at least 25%, such as at least 28%, of the molar content of the HMOs produced by the method is LNDFH-III.

In embodiments the initial substrate is selected from lactose or LNT-II. If the the initial substrate is LNT-II the cell expresses an enzyme with β-1,3-N-acetyl-glucosaminyltransferase activity. Preferably, the initial substrate is lactose and the cell expresses an enzyme with β-1,3-N-acetyl-glucosaminyltransferase activity and an enzyme with β-1,4-galactosyltransferase activity.

A further embodiment of the invention relates to a method for producing LNDFH-III and one or more additional HMOs, comprising
a. providing a genetically engineered cell capable of producing LNDFH-III, comprising a recombinant nucleic acid sequence encoding an α-1,3-fucosyltransferase with dual α-1,3-fucosyltransferase specificity, which is capable of fucosylating an oligosaccharide at a GlcNAc moiety and a Glu moiety, wherein the glycosyltransferase is selected from the group consisting of,
   i. Csec1 comprising or consisting of the amino acid sequence of SEQ ID NO: 7, or a functional homologue thereof with an amino acid sequence that is at least 80% identical to SEQ ID NO: 7,
   ii. CafC comprising or consisting of the amino acid sequence of SEQ ID NO: 8, or a functional homologue thereof with an amino acid sequence that is at least 80% identical to SEQ ID NO: 8,
   iii. FutA variants comprising substitutions at positions corresponding to position 128, 129 of SEQ ID NO: 11, wherein the variants have at least 80% identity, but less than 100% to SEQ ID NO: 11 and
   iv. FucT109, comprising or consisting of the amino acid sequence of SEQ ID NO: 10, or a functional homologue thereof with an amino acid sequence that is at least 80% identical to SEQ ID NO: 10,
      wherein said cell further comprises one or more recombinant nucleic acid sequence(s) encoding a β-1,3-N-acetylglucosaminyltransferase and/or a β-1,4-galactosyltransferase, and
b. culturing the cell in a suitable medium, and
   wherein, at least 20 molar %, such as at least 25 molar %, of the total HMO produced is LNDFH-III, and less than 45 molar % of the total HMO produced is LNFP-VI.

In embodiments, the fucosylated HMOs is LNDFH-III. In further embodiments one or more HMOs selected form the groups consisting of 3FL, LNFP-III, LNFP-VI, LNDFH-III, LNnT, LNT-II and pLNnH are produced by the method of the invention.

The methods comprising cultivating a genetically engineered cell that produces a fucosylated HMO and further comprises culturing said genetically engineered cell in in the presence of a carbon source (energy source), such as a carbon source selected from the group consisting of glucose, sucrose, fructose, xylose and glycerol.

The method particularly comprises cultivating a genetically engineered cell that produces a fucosylated HMO, wherein the LNDFH-III content produced by said cell is at least 25%, such as at least 28%, of the total HMO content produced by the cell.

The method particularly comprises cultivating a genetically engineered cell that produces a fucosylated HMO, wherein at least 60 molar %, such as at least 70 molar %, 75 molar %, 80 molar %, 85 molar %, 90 molar %, 93 molar %, 95 molar %, 96 molar %, 97 molar % or such as at least 98 molar % of the molar content of the total HMOs produced by said cell is fucosylated HMOs.

The method particularly comprises cultivating a genetically engineered cell that produces a fucosylated HMO, wherein at least 50 molar %, such as at least 54 molar %, 60 molar %, 70 molar %, 75 molar %, 80 molar %, 85 molar %, 90 molar %, or such as at least 94 molar %, or such as between 50 molar % and 90 molar % or such as between 70 molar % and 94 molar % of the molar content of the total HMOs produced by a cell according to the present invention is a mixture of LNDFH-III and 3FL. In one embodiment the the fucosylated HMOs produced are primarily LNDFH-III and 3FL with the sum of other fucosylated HMOs being below 15%, such as below 10% of the total molar content of HMO produced.

The method particularly comprises cultivating a genetically engineered cell that produces a fucosylated HMO, wherein at least 50 molar %, such as at least 54 molar %, 60 molar %, 70 molar %, 75 molar %, 80 molar %, 85 molar %, 90 molar %, or such as at least 94 molar %, or such as between 50 molar % and 90 molar % or such as between 70 molar % and 94 molar % of the molar content of the total HMOs produced by a cell according to the present invention is a mixture of LNDFH-III and LNFP-III.

The method particularly comprises cultivating a genetically engineered cell that produces a fucosylated HMO, wherein at least 60 molar %, such as at least 65 molar %, 70 molar %, 75 molar %, 80 molar %, 85 molar %, or such as at least 89 molar %, or such as between 60 molar % and 90 molar % of the molar content of the total HMOs produced by a cell according to the present invention is a mixture of LNDFH-III and LNFP-VI.

The method particularly comprises cultivating a genetically engineered cell that produces a fucosylated HMO, wherein at least 55 molar %, such as at least 59 molar %, 65 molar %, 68 molar %, 70 molar %, 75 molar %, 70 molar %, or such as at least 85 molar %, or such as between 59 molar % and 86 molar % of the molar content of the total HMOs produced by a cell according to the present invention is a mixture of LNDFH-III, LNFP-III and LNFP-VI.

In embodiments, the method according to the present disclosure produces a mixture of HMO(s), wherein the produced mixture of HMOs is essentially free of LNFP-III.

In embodiments, the method according to the present disclosure produces a mixture of HMO(s), wherein the produced mixture of HMOs is essentially free of LNFP-VI.

In embodiments, the method according to the present disclosure produces a mixture of HMO(s), wherein the produced mixture of HMOs is essentially free of LNnT.

In embodiments, the method according to the present disclosure produces a mixture of HMO(s), wherein the produced mixture of HMOs is essentially free of LNT-II.

In embodiments, the method according to the present disclosure produces a mixture of HMO(s), wherein the produced mixture of HMOs is essentially free of 3FL.

The HMO mixtures produced by the methods disclosed herein can be described by their ratios in a mixture of HMOs. The “ratio” as described herein is understood as the ratio between two amounts of HMOs, such as, but not limited to, the amount of one HMO divided by the amount of the other HMO, i.e., the ratio of LNDFH-III:LNFP-III of 2:1 indicates that there is 2 times more LNDFH-III than LNFP-III in the mixture.

In embodiments, the method according to the present disclosure produces a mixture of HMO with an LNDFH-III:LNFP-III ratio of 1:1 to 3:1. In embodiments, the method according to the present disclosure produces a mixture of HMO with an LNDFH-III:LNFP-VI ratio above 1.3, such as above 1.5, such as above 5, such as above 50, such as above 100, such as above 200. In one embodiment, the method according to the present disclosure produces a mixture of HMO(s), selected from the group consisting of i) LNDFH-III and 3FL, ii) LNDFH-III, LNFP-Ill and 3FL, iii) LNDFH-III, LNFP-VI and 3FL, iv) LNDFH-III, LNFP-III, 3FL and LNnT, v) LNDFH-III, LNFP-III, LNFP-VI and 3FL and vi) LNDFH-III, LNFP-III, LNFP-VI, 3FL and LNnT.

The method described herein comprises providing a glycosyl donor, which is synthesized separately by one or more genetically engineered cells and/or is exogenously added to the culture medium from an alternative source. Preferably, the glucosyl donor is produced by an endogenous or recombinant de novo pathway in the genetically engineered cell.

One aspect, the method described herein further comprises providing an acceptor saccharide as initial substrate for the HMO formation, the acceptor saccharide comprising at least two monosaccharide units, which is exogenously added to the culture medium and/or has been produced by a separate microbial fermentation. As an alternative to adding the initial substrate for the production of the HMO to the fermentation medium, the genetically modified cell may be further engineered to produce the initial substrate inside the cell (see for example WO2015/150328).

In one aspect, the method described herein comprises providing an acceptor saccharide comprising at least two monosaccharide units which is selected form lactose, LNT-II and LNnT, and added prior to and/or during the cultivation of the genetically modified cell. In a preferred embodiment the initial substrate for HMO formation is lactose which is fed to the culture during the fermentation of the genetically engineered cell.

The fucosylated human milk oligosaccharide (HMO) is retrieved from the culture, either from the culture medium and/or the genetically engineered cell.

### Culturing/Fermenting

Culturing, cultivating, or fermenting or fermentation (used interchangeably herein) in a controlled bioreactor typically comprises (a) a first phase of exponential cell growth in a culture medium ensured by a carbon-source, and (b) a second phase of cell growth in a culture medium run under carbon limitation, where the carbon-source is added continuously together with the acceptor oligosaccharide, such as lactose, allowing formation of the HMO product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

The terms “manufacturing” or “manufacturing scale” or “large-scale production” or “large-scale fermentation”, are used interchangeably and in the meaning of the invention defines a fermentation with a minimum volume of 100 L, such as 1000 L, such as 10.000 L, such as 100.000 L, such as 200.000 L culture broth. Usually, a “manufacturing scale” process is defined by being capable of processing large volumes yielding amounts of the HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behaviour of an expression system in a lab scale method, such as shake flasks, benchtop bioreactors or the deep well format described in the examples of the disclosure, does allow to predict the behaviour of that system in the complex environment of a bioreactor.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon-source can be selected from the group consisting of glucose, sucrose, fructose, xylose and glycerol. In one or more exemplary embodiments, the culturing media is supplemented with one or more energy and carbon sources selected form the group containing glycerol, sucrose and glucose. In additional embodiments, lactose is added during the cultivation of the genetically engineered cells as a substrate for the HMO formation.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons as described in WO2015/197082 (hereby incorporated by reference).

After carrying out the method of this invention, the fucosylated HMO produced can be collected from the cell culture or fermentation broth in a conventional manner.

### Retrieving/Harvesting

The fucosylated human milk oligosaccharide (HMO) is retrieved from the culture medium and/or the genetically engineered cell. In the present context, the term “retrieving” is used interchangeably with the term “harvesting”. Both “retrieving” and “harvesting” in the context relate to collecting the produced HMO(s) from the culture/broth following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation broth) include extraction thereof from the biomass (i.e., the production cells).

After recovery from fermentation, HMO(s) are available for further processing and purification.

The HMOs can be purified according to the procedures known in the art, e.g., such as described in WO2017/152918, WO2017/182965 or WO2015/188834, wherein the latter describes purification of fucosylated HMOs. The purified HMOs can be used as nutraceuticals, pharmaceuticals, or for any other purpose, e.g., for research.

At the end of culturing, the oligosaccharide as product can be accumulated both in the intra- and the extracellular matrix.

The method according to the present invention comprises cultivating the genetically engineered microbial cell in a culture medium which is designed to support the growth of microorganisms, and which contains one or more carbohydrate sources or just carbon-source, such as selected from the group consisting of glucose, sucrose, fructose, xylose and glycerol. In one or more exemplary embodiments, the culturing media is supplemented with one or more energy and carbon sources selected form the group containing glycerol, sucrose and glucose.

### Manufactured Product

The term “manufactured product” refers to the one or more HMOs intended as the one or more product HMO(s), or composition of a mixture of HMOs. Preferably, the product HMOs or composition is produced by a method described herein using a genetically engineered cell described herein.

Accordingly, an embodiment of the disclosure relates to a mixture of HMOs consisting essentially of
a. LNDFH-III and 3FL, or
b. LNDFH-III, LNFP-III and 3FL, or
c. LNDFH-III, LNFP-VI and 3FL, or
d. LNDFH-III, LNFP-III, 3FL and LNnT, or
e. LNDFH-III, LNFP-III, LNFP-VI, 3FL and LNnT.

One embodiment relates to a composition of HMOs consisting essentially of 20-70 molar % of LNDFH-III, 0-35 molar % LNFP-III, 0-35 molar % LNDFP-VI, 0-65 molar % 3FL, 0-40% LNnT, and at the most 1% pLNnH, in total adding up to 100% molar content.

Another embodiment relates to a composition of HMOs consisting essentially of 35-60 molar % of LNDFH-III and 40-65 molar % 3FL, in total adding up to 100% molar content.

Another embodiment relates to a composition of HMOs consisting essentially of 40-50 molar % LNDFH-III, 25-35 molar % LNFP-VI, 10-20 molar % LNnT and 5-15 molar % 3FL, in total adding up to 100% molar content.

Another embodiment relates to a composition of HMOs consisting essentially of 25-35 molar % LNDFH-III, 15-25 molar % LNFP-VI, 8-18 molar % LNFP-III and 33-43 molar % LNnT, in total adding up to 100% molar content.

Another embodiment relates to a composition of HMOs consisting essentially of 40-50 molar % LNDFH-III, 5-15 molar % LNFP-VI, 3-8 molar % LNFP-III and 2-12 molar % LNnT and 30-40 molar % 3FL, in total adding up to 100% molar content.

Another embodiment relates to a composition of HMOs consisting essentially of 55-65 molar % LNDFH-III, 17-27 molar % LNFP-III and 6-16 molar % LNnT and 1-11 molar % 3FL, in total adding up to 100% molar content. In a preferred embodiment the mixture of HMO consists of 60 molar % LNDFH-I, 20 molar % LNFP-III, 10 molar % LNnT and 10 molar % 3FL.

Another embodiment relates to a composition of HMOs consisting essentially of 36-46 molar % LNDFH-III, 25-35 molar % LNFP-VI, 10-20 molar % LNFP-III and 2-12 molar % LNnT and 2-12 molar % 3FL, in total adding up to 100% molar content.

Another embodiment relates to a composition of HMOs consisting essentially of 50-70 molar % LNDFH-III, 1-13 molar % LNFP-III and 20-50% 3FL, in total adding up to 100% molar content. In a preferred embodiment the mixture of HMO consists of 70 molar % LNDFH-I, 10 molar % LNFP-III and 20 molar % 3FL.

Another embodiment relates to a composition of HMOs consisting essentially of 25-35 molar % LNDFH-III, 29-39 molar % LNFP-VI and 31-41% 3FL, in total adding up to 100% molar content.

Another embodiment relates to a composition of HMOs consisting essentially of 36-46 molar % LNDFH-III, 10-40 molar % LNFP-III and 2-20 molar % LNnT and 2-15 molar % 3FL, in total adding up to 100% molar content. In a preferred embodiment the mixture of HMO consists of 45 molar % LNDFH-I, 30 molar % LNFP-III, 15 molar % LNnT and 10 molar % 3FL.

Another embodiment relates to a composition of HMOs consisting essentially of 37-47 molar % LNDFH-III, 26-36 molar % LNFP-III, 19-29 molar % 3FL, and less than 5% LNnT, in total adding up to 100% molar content.

Another embodiment relates to a composition of HMOs consisting essentially of 19-29 molar % LNDFH-III, 13-23 molar % LNFP-VI, 22-32 molar % LNFP-III and 24-34 molar % LNnT and less than 5 molar % 3FL, in total adding up to 100% molar content.

In further embodiments, a composition or a mixture consists essentially of fucosylated HMOs, e.i., at least 80 molar %, such as at least 90 molar 5, such as at least 95 molar %, such as at least 98 molar % of the total amount of HMO in the composition is fucosylated.

In embodiments, a composition or a mixture of fucosylated consists essentially of LNFP-III, LNFP-VI and/or 3FL, in addition to LNDFH-III.

Advantageously, the methods disclosed herein provide valuable mixtures of HMOs with high levels of fucosylated HMOs including the complex HMO LNDFH-III. Some of the genetically engineered cells described herein produce sufficient LNDFH-III, such as above 20% of the total HMO, to facilitate it purification from the mixture of HMOs produced by the cultivation. In particular genetically engineered cells that produce LNFP-III and/or LNFP-VI at a molar % of the total HMO that is below 10% is highly suitable for purification of LNDFH-III.

The manufactured product may be a powder, a composition, a suspension, or a gel comprising one or more HMOs.

### Use of Composition of or Mixtures of HMOs

Naturally occurring in breast milk, HMOs have evolved over thousands of years, with HMO research (clinical and preclinical) now suggesting that specific HMOs at the correct level of supplementation can provide unique health benefits. As fucosylated HMOs constitute more than 60% of the total HMOs in human milk, mixtures with a high content of fucosylated HMOs are more desirable.

Accordingly, LNDFH-III and mixtures of HMOs comprising LNDFH-III are highly relevant as either a nutritional supplement or as a therapeutic.

Clinical data in infants indicate that Human Milk Oligosaccharide supplements may help to develop the desired microbiota by serving as a food source for the beneficial bacteria in the intestine. In particular, Human Milk Oligosaccharide supplements may help support immunity and gut health, with a potential role in cognitive development, which may open future innovation opportunities.

An aspect of the present disclosure relates to the use of a mixture or composition disclosed herein in infant nutrition.

The present disclosure also relates to the use of a mixture or composition disclosed herein as a dietary supplement or medical nutrition or a pharmaceutical composition.

The mixtures or composition of HMOs may be used to enhance the beneficial bacteria in the gut microbiome. Beneficial bacteria are for example bacteria of the Bifidobacterium sp., lactobacillus sp. or Barnesiella sp. The enhancement of beneficial bacteria may in turn lead to increased production of short chain fatty acids (SCFAs) such as acetate, propionate and butyrate which have been shown to have many benefits in infants and young children, such as inhibition of pathogen bacteria, prevention of infection and diarrhoea, reduced risk of allergy and metabolic disorders (see for example WO2006/130205, WO 2017/129644, WO2017/129649).

The mixtures or composition of HMOs produced according to the method described herein may be used to reduce the abundance of undesirable viruses and bacteria in the gut microbiome. Examples of pathogenic bacteria and viruses that may be reduced by the HMO mixtures described herein are including Candida albicans, Clostridium difficile, Enterococcus faecium, Escherichia coli, Helicobacter pylori, Streptococcus agalactiae, Shigella dysenteriae, Staphylococcus aureus, nora virus and rota virus. Each composition described herein can also be used to treat and/or reduce the risk of a broad range of bacterial infections of a human.

The mixtures or composition of HMOs produced according to the method described herein may be used to increase the regeneration and viability of lyophilized probiotics, including probiotics of Bifidobacterium sp, lactobacillus sp. in particular increased regeneration and/or viability and/or shelf-life in an acidic environment, such as the stomach or acidic food products, is an advantage using the HMO mixtures described herein. Examples of Bifidobacterium sp which may have increased regeneration and viability are Bifidobacterium animals lactis BB12 DSM 32269, Bifidobacterium animals lactis BIF6, Bifidobacterium longum DSM 32946, Bifidobacterium longum BB536, Bifidobacterium bifidum DSMZ 32403, Bifidobacterium infantis, Bifidobacterium breve DSM 33789, Bifidobacterium infantis SP37 DSM 32687, Bifidobacterium adolescentis DSM 34065 and/or Bifidobacterium animalis ssp. animalis DSM 16284. Examples of lactobacillus sp which may have increased regeneration and viability are Lactobacillus rhamnosus GG DSM 32550, Lactobacillus rhamnosus 19070-2 DSM 26357, Lactobacillus rhamnosus GG, Lactobacillus rhamnosus LBrGG ATCC53103, Lactobacillus Probio-Tec® LGG® DSM 33156, Lactobacillus reuteri DSM 12246, Lactobacillus plantarum TIFN101, Lactobacillus gasseri Lg-36 200B FloraFit Danisco, Lactobacillus casei DSM 32382, Lactobacillus paracasei, Lactobacillus paracasei L26-CBS116412. Lactobacillus plantarum PS 128, Lactobacillus plantarum (Sacco) DSM 32383, Lactococcus lactis PAREVE, and/or Limosilactobacillus reuteri S12 DSM 33752.

In the context of the present application “Regeneration” means the process of regaining/restoring a dried bacteria's viability (i.e., “reviving” the bacterial cells by rehydration, wherein “rehydration” means restoring fluid). This process is also sometimes referred to as “reconstitution”.

In the context of the present application “viability” is the ability of a bacterial cell to live and function as a living cell. One way of determining the viability of bacterial cells is by spreading them on an agar plate with suitable growth medium and counting the number of colonies formed after incubation for a predefined time (plate counting). Alternatively, FACS analysis may be used.

In the context of the present application “Improving the regeneration” of Bifidobacterium sp and/or Lactobacillus sp bacteria means to increase the amount (number) of Bifidobacterium sp and/or Lactobacillus sp. bacteria successfully regenerating/reviving compared to the respective control (i.e., the amount/number of Bifidobacterium sp and/or Lactobacillus sp. bacteria without the addition of HMO).

An embodiment of the present disclosure is the use of a composition of HMOs consisting essentially of one of the following mixtures
a LNDFH-III and 3FL, or
b. LNDFH-III, LNFP-III and 3FL, or
c. LNDFH-III, LNFP-III, 3FL and LNnT, or
   to regenerate or revive a probiotic strain of the species Bifidobacterium sp and/or lactobacillus sp. Preferably, the probiotic strain is selected from of the species Lactobacillus rhamnosus sp., Lactobacillus paracasei sp and/or Bifidobacterium adolescentis sp., such as Lactobacillus rhamnosus DSM 33156, Lactobacillus rhamnosus ATCC53103, Lactobacillus paracasei L26-CBS116412 and/or Bifidobacterium adolescentis DSM 34065.

In the context of the present application “Improving the viability” of Bifidobacterium sp and/or Lactobacillus sp bacteria means to increase the amount (number) of viable Bifidobacterium sp and/or Lactobacillus sp. bacteria compared to the respective control (i.e., the amount/number of Bifidobacterium sp and/or Lactobacillus sp. bacteria without the addition of HMO).

In the context of the present application “acidic” means having a pH below 7.0 (for example, having a pH≤6.0, or ≤5.0, or ≤4.0, or ≤3.0, or in the range of 1.0-6.0, such as from 2.0 to 5.0). The pH measured in the stomach is in the range of about 1.5-3.5. The pH measured in a healthy vagina is in the range of about 3.8-5.0. The pH of fruit juices is in the range of about 2.0-4.5.

The mixtures or composition of HMOs produced according to the method described herein or otherwise described herein, may be used to extend the shelf life of probiotics, such as Bifidobacterium sp, and/or Lactobacillus sp.

A composition comprising a combination of prebiotics (e.g. HMOs) and probiotics are generally termed synbiotics or synbiotic compositions. Preferably, a synbiotic composition provide an additional, preferably a synergistic effect as compared to the effect of the individual prebiotics and probiotics.

An embodiment of the present invention is a synbiotic composition comprising one or more probiotics and a mixture of HMOs as described herein, in particular in the section “Manufactured product”. Preferably, the one or more probiotics is a Bifidobacterium sp and/or Lactobacillus sp such as any of the specific species mentioned above.

One embodiment of the present disclosure is a synbiotic composition comprising an HMO mixture consisting essentially of one of the following mixtures
a. LNDFH-III and 3FL, or
b. LNDFH-III, LNFP-III and 3FL, or
C. LNDFH-III, LNFP-III, 3FL and LNnT, or
   in combination with a probiotic strain selected from Bifidobacterium and/or Lactobacillus species. Preferably, of the probiotic strain is selected from one or more of the species Lactobacillus rhamnosus sp., Lactobacillus paracasei sp and/or Bifidobacterium adolescentis sp., such as Lactobacillus rhamnosus DSM 33156, Lactobacillus rhamnosus ATCC53103, Lactobacillus paracasei L26-CBS116412 and/or Bifidobacterium adolescentis DSM 34065.

In a further embodiment the synbiotic composition comprises or consists of a probiotic selected from the group consisting of Lactobacillus rhamnosus sp., Lactobacillus paracasei sp and/or Bifidobacterium adolescentis sp. and a mixture of HMOs consisting essentially of 40-70 molar % of LNDFH-III, 0-35 molar % LNFP-III, 0-35 molar % LNFP-VI, 5-55 molar % 3FL, 0-20% LNnT, and below 1% pLNnH, in total adding up to 100% molar content of HMO in the composition.

In one embodiment the synbiotic composition comprises or consists of a probiotic selected from the group consisting of Lactobacillus rhamnosus sp., Lactobacillus paracasei sp and/or Bifidobacterium adolescentis sp. and a mixture of HMOs selected from the group consisting of:
a. a mixture consisting essentially of 35-60 molar % of LNDFH-III and 40-65 molar % 3FL, in total adding up to 100% molar content,
b. a mixture consisting essentially of 50 molar % of LNDFH-III and 50 molar % 3FL,
c. a mixture consisting essentially of 40-50 molar % LNDFH-III, 25-35 molar % LNFP-VI, 10-20 molar % LNnT and 5-15 molar % 3FL, in total adding up to 100% molar content,
d. a mixture consisting essentially of 25-35 molar % LNDFH-III, 15-25 molar % LNFP-VI, 8-18 molar % LNFP-III and 33-43 molar % LNnT, in total adding up to 100% molar content,
e. a mixture consisting essentially of 40-50 molar % LNDFH-III, 5-15 molar % LNFP-VI, 3-8 molar % LNFP-III and 2-12 molar % LNnT and 30-40 molar % 3FL, in total adding up to 100% molar content,
f. a mixture consisting essentially of 55-65 molar % LNDFH-III, 17-27 molar % LNFP-III and 6-16 molar % LNnT and 1-11 molar % 3FL, in total adding up to 100% molar content,
g. a mixture consisting essentially of 60 molar % LNDFH-III, 20 molar % LNFP-III and 10 molar % LNnT and 10 molar % 3FL,
h. a mixture consisting essentially of 36-46 molar % LNDFH-III, 25-35 molar % LNFP-VI, 10-20 molar % LNFP-III and 2-12 molar % LNnT and 2-12 molar % 3FL, in total adding up to 100% molar content,
i. a mixture consisting essentially of 50-70 molar % LNDFH-III, 1-13 molar % LNFP-III and 20-50% 3FL, in total adding up to 100% molar content,
j. a mixture consisting essentially of 70 molar % LNDFH-III, 10 molar % LNFP-III and 20-50% 3FL,
k. a mixture of consisting essentially of 36-46 molar % LNDFH-III, 25-35 molar % LNFP-VI, 10-20 molar % LNFP-III and 2-12 molar % LNnT and 2-12 molar % 3FL, in total adding up to 100% molar content,
l. a mixture consisting essentially of 45 molar % LNDFH-III, 30 molar % LNFP-III, 10 molar % 3FL, and 15% LNnT,
m. a mixture consisting essentially of 25-35 molar % LNDFH-III, 29-39 molar % LNFP-VI and 31-41% 3FL, in total adding up to 100% molar content,
n. a mixture consisting essentially of 37-47 molar % LNDFH-III, 26-36 molar % LNFP-III, 19-29 molar % 3FL, and less than 5% LNnT, in total adding up to 100% molar content, and
o. a mixture consisting essentially of 19-29 molar % LNDFH-III, 13-23 molar % LNFP-VI, 22-32 molar % LNFP-III and 24-34 molar % LNnT and less than 5 molar % 3FL, in total adding up to 100% molar content.

Preferably, the probiotic selected from the following strains Lactobacillus rhamnosus Probio-Tec® LGG®-DSM 33156, Lactobacillus rhamnosus LBrGG-ATCC53103, Lactobacillus paracasei L26-CBS116412 and/or Bifidobacterium adolescentis DSM 34065.

The mixtures or composition of HMOs described herein, may be used to improve the flowability of a powder or decrease the viscosity of a liquid.

Composition and mixtures of HMOs described in the section “Manufactured product” may also form part of a composition comprising additional parts, such as active pharmaceutical ingredients, food supplements, excipients, surfactants etc.

Accordingly, embodiments described herein relate to the use of a composition comprising a mixture of HMOs produced according to the present invention as a dietary supplement or medical nutrition. In further embodiments the composition for use in an infant formula, a dietary supplement or medical nutrition comprises LNDFH-III, 3FL, LNFP-III, and LNFP-VI.

The mixtures or composition of HMOs described herein are used in a nutritional composition. Nutritional compositions are for example, an infant formula, a rehydration solution, or a dietary maintenance, medical nutrition or supplement for elderly individuals or immunocompromised individuals. Macronutrients such as edible fats, carbohydrates and proteins can also be included in such anti-infective compositions. Edible fats include, for example, coconut oil, soy oil and monoglycerides and diglycerides. Carbohydrates include, for example, glucose, edible lactose and hydrolysed cornstarch. Proteins include, for example, soy protein, whey, and skim milk. Vitamins and minerals (e. g. calcium, phosphorus, potassium, sodium, chloride, magnesium, manganese, iron, copper, zinc, selenium, iodine, and Vitamins A, E, D, C, and B complex) can also be included in such anti-infective compositions.

In embodiments, the composition comprising a mixture of HMOs produced according to the present invention is a pharmaceutical composition.

The present invention also relates to the use of a mixture or composition according to the present invention as a dietary supplement and/or medical nutrition.

In embodiments, the invention relates to the use of a mixture or composition according to the present invention in infant nutrition.

### ITEMS

Various embodiments of present disclosure are described in the following items
1. A genetically engineered cell capable of producing LNDFH-III, comprising a recombinant nucleic acid sequence encoding an α-1,3-fucosyltransferase selected from the group consisting of,
   a. Osc1 comprising or consisting of the amino acid sequence of SEQ ID NO: 1, or a functional homologue thereof with an amino acid sequence that is at least 80% identical to SEQ ID NO: 1,
   b. Bgall1 comprising or consisting of the amino acid sequence of SEQ ID NO: 2, or a functional homologue thereof with an amino acid sequence that is at least 80% identical to SEQ ID NO: 2,
   c. Bbac1 comprising or consisting of the amino acid sequence of SEQ ID NO: 3, or a functional homologue thereof with an amino acid sequence that is at least 80% identical to SEQ ID NO: 3,
   d. Murba1 comprising or consisting of the amino acid sequence of SEQ ID NO: 4, or a functional homologue thereof with an amino acid sequence that is at least 80% identical to SEQ ID NO: 4,
   e. Bacfin1 comprising or consisting of the amino acid sequence of SEQ ID NO: 5, or a functional homologue thereof with an amino acid sequence that is at least 80% identical to SEQ ID NO: 5,
   f. Prev1 comprising or consisting of the amino acid sequence of SEQ ID NO: 6, or a functional homologue thereof with an amino acid sequence that is at least 80% identical to SEQ ID NO: 6,
   g. Csec1 comprising or consisting of the amino acid sequence of SEQ ID NO: 7, or a functional homologue thereof with an amino acid sequence that is at least 80% identical to SEQ ID NO: 7,
   h. CafC comprising or consisting of the amino acid sequence of SEQ ID NO: 8, or a functional homologue thereof with an amino acid sequence that is at least 80% identical to SEQ ID NO: 8 and
   i. FutA variants comprising substitutions at a position corresponding to position 128 and 129 of SEQ ID NO: 11, wherein the variants have at least 80% sequence identity to SEQ ID NO: 11, and
      wherein the cell further comprises one or more recombinant nucleic acid sequence(s) encoding a β-1,4-galactosyltransferase and optionally a β-1,3-N-acetylglucosaminyltransferase.
2. The genetically engineered cell according to any one item 1, wherein at least 28% of the molar content of the total HMOs produced by said cell is LNDFH-III.
3. The genetically engineered cell according to item 1 or 2, wherein the cell further produces one or more HMOs selected from the group consisting of 3FL, LNnT, LNFP-III and LNFP-VI.
4. The genetically engineered cell according to to any of the preceding claims, wherein the cell further comprises one or more recombinant nucleic acid sequence(s) encoding a β-1,3-N-acetylglucosaminyltransferase and/or a β-1,4-galactosyltransferase.
5. The genetically engineered cell according to any of the preceding items, wherein the cell further comprises a substrate importer selected from a lactose importer, a lacto-N-triose-II (LNT-II) importer or a LNnT importer.
6. The genetically engineered cell according to any of the preceding items, wherein the recombinant nucleic acid sequences individually are under the control of one or more promoters selected from the group consisting of PglpF, Plac, PmglB_70UTR, PglpA_70UTR and PglpT_70UTR (SEQ ID NOs: 39, 48, 36, 37 and 38) and variants thereof.
7. The genetically engineered cell according to any of the preceding items, wherein the engineered cell is a microorganism.
8. The genetically engineered cell, wherein the engineered cell is a prokaryotic cell or eukaryotic cell.
9. The genetically engineered cell according to item 7 or 8, wherein the engineered cell is a fungal host selected from yeast cells, such as Komagataella, Kluyveromyces, Yarrowia, Pichia, Saccaromyces, Schizosaccharomyces or Hansenula or from a filamentous fungus such as Aspargillus, Fusarium or Thricoderma.
10. The genetically engineered cell according to item 7 or 8, wherein the engineered cell is a bacterial cell selected from the group consisting of Escherichia sp., Bacillus sp., Lactobacillus sp., Corynebacterium sp. and Campylobacter sp.
11. The genetically engineered cell according to item 7 or 8, wherein said engineered cell is selected from the group consisting of Escherichia coli, Bacillus subtilis, Lactobacillus lactis, Corynebacterium glutamicum, Yarrowia lipolytica, Pichia pastoris, and Saccharomyces cerevisiae.
12. A method for producing one or more fucosylated HMOs comprising providing and culturing a genetically engineered cell comprising a recombinant nucleic acid sequence encoding an α-1,3-fucosyltransferase selected from the group consisting of,
   a. Osc1 comprising or consisting of the amino acid sequence of SEQ ID NO: 1, or a functional homologue thereof with an amino acid sequence that is at least 80% identical to SEQ ID NO: 1,
   b. Bgall1 comprising or consisting of the amino acid sequence of SEQ ID NO: 2, or a functional homologue thereof with an amino acid sequence that is at least 80% identical to SEQ ID NO: 2,
   c. Bbac1 comprising or consisting of the amino acid sequence of SEQ ID NO: 3, or a functional homologue thereof with an amino acid sequence that is at least 80% identical to SEQ ID NO: 3,
   d. Murba1 comprising or consisting of the amino acid sequence of SEQ ID NO: 4, or a functional homologue thereof with an amino acid sequence that is at least 80% identical to SEQ ID NO: 4,
   e. Bacfin1 comprising or consisting of the amino acid sequence of SEQ ID NO: 5, or a functional homologue thereof with an amino acid sequence that is at least 80% identical to SEQ ID NO: 5,
   f. Prev1 comprising or consisting of the amino acid sequence of SEQ ID NO: 6, or a functional homologue thereof with an amino acid sequence that is at least 80% identical to SEQ ID NO: 6,
   g. Csec1 comprising or consisting of the amino acid sequence of SEQ ID NO: 7, or a functional homologue thereof with an amino acid sequence that is at least 80% identical to SEQ ID NO: 7,
   h. CafC comprising or consisting of the amino acid sequence of SEQ ID NO: 8, or a functional homologue thereof with an amino acid sequence that is at least 80% identical to SEQ ID NO: 8,
   i. FucT109 comprising or consisting of the amino acid sequence of SEQ ID NO: 10, or a functional homologue thereof with an amino acid sequence that is at least 80% identical to SEQ ID NO: 10, and
   j. FutA variants comprising substitutions at a position corresponding to position 128 and 129 of SEQ ID NO: 11, wherein the variants have at least 80% sequence identity to SEQ ID NO: 11, and wherein at least one of the fucosylated HMOs is LNDFH-III.
13. The method according to item 12, said method comprising providing and culturing a genetically engineered cell according to any one of items Error! Reference source not found. to 11.
14. The method according to item 12 or 13, wherein the culturing is done in a suitable cell culture medium to produce said one or more fucosylated HMOs, wherein at least one of the fucosylated HMOs is LNDFH-III.
15. The method according to item 12 to 14, wherein said one or more fucosylated HMOs are purified.
16. The method according to any one of items 12 to 15, wherein at least 25% of the molar content of the total HMOs produced by said method is LNDFH-III.
17. The method according to any of items 12 to 16, wherein at least one additional fucosylated HMOs selected from the group consisting of 3FL, LNFP-III and LNFP-VI is produced.
18. The method according to any of items 12 to 17, wherein the LNDFH-III:LNFP-VI ratio produced is above 1.3.
19. The method according to any of items 12 to 17, wherein the LNDFH-III:LNFP-III ratio is between 1:1 to 3:1
20. The method according to any of items 12 to 17, wherein less than 10% of the molar content of the total HMOs produced is LNFP-III and less than 10% of the molar content of the total HMOs produced is LNFP-VI.
21. The method according to any of items 12 to 17, wherein the fucosylated HMOs produced are primarily LNDFH-III and 3FL with the sum of other fucosylated HMOs being below 15%, such as below 10% of the total molar content of HMO produced.
22. The method according to any one of items 12 to 21, wherein the genetically engineered cell is cultivated in the presence of an acceptor substrate selected from the group consisting of lactose, LNT-II and LNnT.
23. Use of an α-1,3-fucosyltransferase in the production of one or more fucosylated HMOs, wherein the enzyme is selected from the group consisting of Osc1, Bgall1, Bbac1, Murba1, Bacfin1, Prev1, Csec1, CafC and FutA_mut2 comprising or consisting of the amino acid sequence of SEQ ID NO: 1, 2, 3, 4, 5, 6, 7, 8 or 9, or a functional homologue thereof with an amino acid sequence that is at least 80% identical to SEQ ID NO: 1, 2, 3, 4, 5, 6, 7, 8 or 9, or a FutA variant comprising substitutions at positions 128, 129 of SEQ ID NO: 11, wherein the variants has at least 80% identity, but less than 100% to SEQ ID NO: 11.
24. A mixture of HMOs produced with a method according to any of items 12 to 22, consisting essentially of
   a. LNDFH-III and 3FL, or
   b. LNDFH-III, LNFP-III and 3FL, or
   c. LNDFH-III, LNFP-VI and 3FL, or
   d. LNDFH-III, LNFP-III, LNFP-VI and LNnT, or
   e. LNDFH-III, LNFP-III, 3FL and LNnT, or
   f. LNDFH-III, LNFP-III, LNFP-VI, 3FL and LNnT.
25. A composition of HMOs consisting essentially of 20-70 molar % of LNDFH-III, 0-35 molar % LNFP-III, 0-35 molar % LNFP-VI, 0-65 molar % 3FL, 0-40% LNnT, and below 1% pLNnH, in total adding up to 100% molar content.
26. The composition according to item 25, wherein the composition comprises a mixture of HMOs selected from the group consisting of:
   a. a mixture consisting essentially of 35-60 molar % of LNDFH-III and 40-65 molar % 3FL, in total adding up to 100% molar content,
   b. a mixture consisting essentially of 50 molar % of LNDFH-III and 50 molar % 3FL,
   c. a mixture consisting essentially of 40-50 molar % LNDFH-III, 25-35 molar % LNFP-VI, 10-20 molar % LNnT and 5-15 molar % 3FL, in total adding up to 100% molar content,
   d. a mixture consisting essentially of 25-35 molar % LNDFH-III, 15-25 molar % LNFP-VI, 8-18 molar % LNFP-III and 33-43 molar % LNnT, in total adding up to 100% molar content,
   e. a mixture consisting essentially of 40-50 molar % LNDFH-III, 5-15 molar % LNFP-VI, 3-8 molar % LNFP-III and 2-12 molar % LNnT and 30-40 molar % 3FL, in total adding up to 100% molar content,
   f. a mixture consisting essentially of 55-65 molar % LNDFH-III, 17-27 molar % LNFP-III and 6-16 molar % LNnT and 1-11 molar % 3FL, in total adding up to 100% molar content,
   g. a mixture consisting essentially of 60 molar % LNDFH-III, 20 molar % LNFP-III and 10 molar % LNnT and 10 molar % 3FL,
   h. a mixture consisting essentially of 36-46 molar % LNDFH-III, 25-35 molar % LNFP-VI, 10-20 molar % LNFP-III and 2-12 molar % LNnT and 2-12 molar % 3FL, in total adding up to 100% molar content,
   i. a mixture consisting essentially of 50-70 molar % LNDFH-III, 1-13 molar % LNFP-III and 20-50% 3FL, in total adding up to 100% molar content,
   j. a mixture consisting essentially of 70 molar % LNDFH-III, 10 molar % LNFP-III and 20-50% 3FL,
   k. a mixture of consisting essentially of 36-46 molar % LNDFH-III, 25-35 molar % LNFP-VI, 10-20 molar % LNFP-III and 2-12 molar % LNnT and 2-12 molar % 3FL, in total adding up to 100% molar content,
   l. a mixture consisting essentially of 45 molar % LNDFH-III, 30 molar % LNFP-III, 10 molar % 3FL, and 15% LNnT,
   m. a mixture consisting essentially of 25-35 molar % LNDFH-III, 29-39 molar % LNFP-VI and 31-41% 3FL, in total adding up to 100% molar content,
   n. a mixture consisting essentially of 37-47 molar % LNDFH-III, 26-36 molar % LNFP-III, 19-29 molar % 3FL, and less than 5% LNnT, in total adding up to 100% molar content, and
   o. a mixture consisting essentially of 19-29 molar % LNDFH-III, 13-23 molar % LNFP-VI, 22-32 molar % LNFP-III and 24-34 molar % LNnT and less than 5 molar % 3FL, in total adding up to 100% molar content.
27. A synbiotic composition comprising a probiotic strain selected from one or more Bifidobacterium and/or Lactobacillus species and a mixture of HMOs according to Item 24 or a composition of HMOs according to items 25 or 26.
28. The synbiotic composition according to item 27, wherein the probiotic strain is selected from one or more of the species Lactobacillus rhamnosus sp., Lactobacillus paracasei sp and/or Bifidobacterium adolescentis sp.
29. The synbiotic composition according to item 27 or 28, wherein the probiotic strain is selected from one or more of the strains Lactobacillus rhamnosus DSM 33156, Lactobacillus rhamnosus ATCC53103, Lactobacillus paracasei L26-CBS116412 and/or Bifidobacterium adolescentis DSM 34065.
30. The synbiotic composition according to item 27 to 29, wherein the pH of the composition is below 5.0.
31. Use of a mixture according to item 24, or an HMO composition according to item 25 or 26, or a synbiotic compositions according to any one of item 27 to 30, in an infant formula, a dietary supplement and/or medical nutrition.

### Sequences

The current application contains a sequence listing in text format and electronical format which is hereby incorporated by reference.

An overview of the SEQ ID NOs used in the present application can be found in table 1 (α-1,3-fucosyltransferase protein sequences (SEQ ID NO:1-11 and 51) and table 4 (promoter sequences SEQ ID NO: 27-50), additional sequences described in the application is the DNA sequences encoding the α-1,3-fucosyltransferases (SEQ ID NO: 12 to 22 and 52), the DNA sequence encoding the colanic acid gene cluster from E. coli (SEQ ID NO: 23) and the β-1,3-N-acetylglucosaminyltransferase LgtA from N. meningitidis (SEQ ID NO: 24), β-1,4-galactosyltransferases galT from H. pylori (SEQ ID NO: 25), and Lactose permease, LacY (SEQ ID NO: 26).

### EXAMPLES

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY)

The embodiments described below are selected to illustrate the invention and are not limiting the invention in any way.

### Enzymes:

Screening of 50 enzymes with fucosyltransferase activity provided ten enzymes that have not previously been shown to process dual α-1,3-fucosyltransferase activity, with an α-1,3-fucosyltransferase activity on the Glucose and α-1,3-fucosyltransferase activity on N-Acetylglucosamine moiety of LNnT, resulting in production of the complex di-fucosylated HMO LNDFH-III. The GenBank ID and origin of the ten α-1,3-fucosyltransferases as well as the prior art, 3-fucosyltransferases, FutA, are provided in table 5.

TABLE 5
List of the enzymes tested in the
framework of the present invention

| Enzyme | | SEQ | |
|---|---|---|---|
| Name | GenBank ID\hat{ } | ID NO: | Origin |
| Osc1 | MBC8592593.1 | 1 | Oscillospiraceae bacterium |
| | | | N12 |
| Bgall1* | WP_204430034.1 | 2 | Bacteroides gallinaceum |
| Bbac1 | MBR5851405.1 | 3 | Bacteroidaceae bacterium |
| Murba1 | MBP3303338.1 | 4 | Muribaculaceae bacterium |
| Bacfin1 | WP_195630400.1 | 5 | Bacteroides finegoldii |
| Prev1 | MBP3220331.1 | 6 | Prevotella sp. |
| Csec1 | KHM48694.1 | 7 | Coprobacter secundus |
| CafC** | WP_007483358.1 | 8 | Bacteroides nordii |
| FutA_{—} | — | 9 | Artificial construct |
| mut2*** | | | |
| FucT109\hat{ }\hat{ } | CAH09151.1 | 10 | Bacteroides fragilis |
| | | | NCTC 9343 |
| FutA\hat{ }\hat{ }\hat{ } | | 11 | Helicobacter pylori |
| FutB\hat{ }\hat{ }\hat{ } | WP_000487430.1 | 51 | Helicobacter pylori |

\hat{ }the sequences used in the present application may be truncated at the N- or C-terminal as compared to the GenBank sequence these are represented by the SEQ ID NO.
*Bgall1 (FutM2) has been shown to produce 3FL in Chen et al., 2022 Arg Food Chem
**CafC has been suggested to produce 3FL, DFL and LNFP-III in WO2016/040531
***FutA_mut2 have been shown to produce LNFP-V in WO2020/115671
\hat{ }\hat{ }FucT109 has been suggested to produce mixtures of LNFP-III and LNFP-VI in WO 2019/008133
\hat{ }\hat{ }\hat{ }FutA and FutB has been shown to produce LNDFH-III in Dumon et al., 2004 (α-1,3-fucosyltransferase, Biotechnol. Prog. 2004, 20, 412-419).

### Strains

The strains (genetically engineered cells) constructed in the present application were based on Escherichia coli K-12 DH1 with the genotype: F⁻, λ, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186:2673-81 and Warming et al 2005 Nucleic Acids Res. 33 (4): e36) with specific selection marker genes and screening methods.

To obtain an LNnT producing strain the MDO strain was further engineered by chromosomally integrating a beta-1,3-GlcNAc transferase (LgtA from Neisseria meningitidis, homologous to NCBI Accession nr. WP_033911473.1 and shown as SEQ ID NO: 24) and a beta-1,4-galactosyltransferase (GalT from Helicobacter pylori, homologous to GenBank ID WP_001262061.1 and shown as SEQ ID NO: 25) both under the control of a PglpF promoter (SEQ ID NO: 39), this strain is named the LNnT strain.

Codon optimized DNA sequences encoding individual α-1,3-fucosyltransferases were genomically integrated into the LNnT strain.

The genotypes of the background strain (MDO), the LNnT strain and the α-1,3/4-fucosyltransferase expressing strains capable of producing LNDFH-III, and mixtures thereof are provided in Table 6.

TABLE 6
Genotypes of the strains, capable of producing
LNDFH-III, used in the present examples.

| | | α-1,3FT* |
|---|---|---|
| Strain | | cDNA |
| ref | Genotype | SEQ ID NO |
| MDO | F− λ− ΔendA1 ΔrecA1 ΔrelA1 ΔgyrA96 | — |
| | Δthi-1 glnV44 hsdR17(rK−mK−) ΔlacZ | |
| | wcaF::Plac ΔnanKETA ΔlacA ΔmelA ΔwcaJ | |
| | ΔmdoH | |
| LNnT | MDO, 2x IgtA-PglpF¹, 1x galT-PglpF² | — |
| Osc1-1 | LNnT, Osc1-PglpF, CA-PglpF³ | 12 |
| Osc1-2 | LNnT, 2xOsc1-PglpF, CA-PglpF³ | 12 |
| Bgall1-1 | LNnT, Bgall1-PglpF, CA-PglpF³ | 13 |
| Bgall1-2 | LNnT, 2x Bgall1-PglpF, CA-PglpF³ | 13 |
| Bbac1 | LNnT, Bbac1-PglpF, CA-PglpF³ | 14 |
| Murba1 | LNnT, Murba1-PglpF, CA-PglpF³ | 15 |
| Csec | LNnT, Csec-PglpF, CA-PglpF³ | 16 |
| Bacfin1 | LNnT, Bacfin1-PglpF, CA-PglpF³ | 17 |
| Prev1 | LNnT, Prev1-PglpF, CA-PglpF³ | 18 |
| CafC | LNnT, CafC-PglpF, CA-PglpF³ | 19 |
| FutA_mut2 | LNnT, FutA_mut2-PglpF, CA-PglpF³ | 20 |
| FucT109 | LNnT, FucT109-PglpF, CA-PglpF³ | 21 |
| FutA | LNnT, FutA-PglpF, CA-PglpF³ | 22 |
| FutB | LNnT, FutB-PglpF, CA-PglpF³ | 52 |

*1,3FT is an abbreviation of α-1,3-fucosyltransferase, and the DNA sequence is inserted into the genome of the host strain.
¹IgtA-PglpF - two genomically inserted copies of a gene encoding β-1,3-N-acetyl-glucosaminyltransferase (SEQ ID NO: 24) under control of a PglpF promoter.
²galT-PglpF - one genomically inserted gene encoding β-1,4-Galactosyltransferase (SEQ ID NO: 25) under control of a PglpF promoter.
³CA = extra colanic acid gene cluster (gmd-wcaG-wcaH-wcal-manC-manB, SEQ ID NO: 23) under the control of a PglpF promoter at a locus that is different than the native locus.

### Deep Well Assay

Deep Well Assays in the current examples were performed as originally described by Lv et al (Bioprocess Biosyst Eng 20 (2016) 39:1737-1747) and optimized for the purposes of the current invention. More specifically, the strains disclosed in the present example were screened in 96 deep well plates using a 4-day protocol. During the first 24 hours, precultures were grown to high densities (OD600 up to 5) and subsequently transferred to a medium that allowed induction of gene expression and product formation.

More specifically, during day 1, fresh precultures were prepared using a basal minimal medium (BMM) (pH 7.0) supplemented with magnesium sulphate (0.12 g/L), thiamine (0.004 g/L) and glucose (5.5 g/L). Basal Minimal medium had the following composition: NaOH (1 g/L), KOH (2.5 g/L), KHzPO4 (7 g/L), NH&HzPO4 (7 g/L), Citric acid (0.5 g/l), trace mineral solution (5 mL/L). The trace mineral stock solution contained; ZnSO~*7H~O 0.82 g/L, Citric acid 20 g/L, Mn$04*H&O 0.98 g/L, FeSO4*7H&O 3.925 g/L, CuSO4*5H~O 0.2 g/L. The pH of the Basal Minimal Medium was adjusted to 7.0 with 5 N NaOH and autoclaved. The precultures were incubated for 24 hours at 34° C. and 1000 rpm shaking and then further transferred to 0.75 ml of a new BMM (pH 7.5) to start the main culture. The new BMM was supplemented with magnesium sulphate (0.12 g/L), thiamine (0.02 g/L), a bolus of glucose solution (0.1-0.15 g/L) and a bolus of lactose solution (5-20 g/L) Moreover, a 20% stock solution of sucrose (40-45 g/L) or maltodextrin (19-20 g/L) was provided as carbon source, accompanied by the addition of a specific hydrolytic enzyme, sucrose hydrolase or glycoamylase, respectively, so that glucose was released at a rate suitable for carbon-limited growth and similar to that of a typical fed-batch fermentation process. The main cultures were incubated for 72 hours at 28° C. and 1000 rpm shaking. For the analysis of total broth, the 96 well plates were boiled at 100° C., subsequently centrifuged, and finally the supernatants were analysed by HPLC.

### Fermentation

The E. coli strains were cultivated in 250 mL fermenters (Ambr250 HT Bioreactor system, Sartorius) starting with 100 mL of mineral culture medium consisting of 30 g/L glucose and a mineral medium comprised of NH₄H₂PO₄, KH₂PO₄, MgSO₄×7H₂O, KOH, NaOH, citric acid, trace element solution, antifoam and thiamine. The dissolved oxygen level was kept at 20% by a cascade of first agitation and then airflow starting at 700 rpm (up to max 4500 rpm) and 1 WVM (up to max 3 VVM). The pH was kept at 6.8 by titration with 8.5% NH₄OH solution. The cultivations were started with 2% (v/v) inoculums from pre-cultures comprised of 10 g/L glucose, (NH₄)₂HPO₄, KH₂PO₄, MgSO₄×7H₂O, KOH, NaOH, citric acid, trace element solution, antifoam and thiamine. After depletion of the glucose contained in the basal minimal medium, a feed solution containing glucose, MgSO₄×7H₂O, H₃PO₄ and trace mineral solution was continuously added to the fermenter at a rate that maintained carbon-limiting conditions. The temperature was initially at 33° C. but was dropped to 30° C. with a 3-hour linear ramp initiated 12 hours after the start of the feed. Lactose was added as bolus additions of 25% lactose monohydrate solution 6 hours after feed start and then every 19 hours to keep lactose from becoming a rate limiting factor. The growth, metabolic activity and metabolic state of the cells was followed by on-line measurements of agitation, dissolved oxygen tension, reflectance, NH₄OH base addition, O₂ uptake rate and CO₂ evolution rate. Throughout the fermentations, samples were taken to determine the concentration of HMO products, lactose and other minor by-products using HPLC.

### Example 1—In Vivo LNDFH-III Synthesis

Genetically modified cells expressing individual α-1,3-fucosyltransferase enzymes (as listed I table 6) were screened for their ability to produce the fucosylated complex HMO LNDFH-III and mixtures thereof.

Ten enzymes (table 7) which to our knowledge have not previously been described to be able to synthesize LNDFH-III when introduced into a genetically modified cells that produce LNnT and GDP-Fucose, were compiled for testing. Some of the enzymes are known to produce other fucosylated HMOs, namely FucT109a, CafC, FutA_mut2, FutA and FutB, which have been suggested in the prior art to be capable of decorating the glucose moiety of lactose, LNnT or LNT or the N-acetylglucosamine moiety of LNnT.

Specifically, FucT109 also known as CafA have been shown to produce LNFP-III, LNFP-VI, 3FL and DFL (WO2019008133 and WO2016040531), CafC have been shown to produce 3FL and DFL (WO2016/040531) and FutA_mut2 have been shown to produce LNFP-V when introduced into an LNT producing strain (WO2020/115671). Bgall1 (FutM2) has been shown to produce 3FL in Chen et al., 2022 Arg Food Chem. None of these have however been shown to produce LNDFH-III.

FutA and FutB have been shown to produce LNDFH-II by Dumon et al., 2004 (Biotechnol. Prog. 2004, 20, 412-419), and are used as reference α-1,3-fucosyltransferases.

Genetically modified strains expressing the 12 individual α-1,3-fucosyltransferases (table 5) were generated as described in the “Method” section. The cells were screened in the deep well assay setup as described in the “Method” section.

Table 6 lists the genotype of the strains capable of producing LNDFH-III. The molar content of individual HMOs produced by the strains was measured by HPLC.

The results of the LNDFH-III producing cells are shown in table 7 as the fraction of the total molar HMO content (in percentage, %) produced by each strain.

TABLE 7
Content of individual HMO's as % of total
HMO molar (mM) content produced by each strain.

| | FT Copy | LNDFH- | LNFP- | LNFP- | | | |
|---|---|---|---|---|---|---|---|
| Enzyme | nr | III | VI | III | LNnT | 3FL | pLNnH |
| Bgall1-1 | 1 | 67.4 | 0.3 | 7.8 | 0.1 | 24.4 | 0.0 |
| Bgall1-2 | 2 | 52.1 | 0.0 | 2.7 | 0.0 | 45.3 | 0.0 |
| Bacfin1 | 1 | 61.4 | 0.3 | 22.1 | 10.5 | 5.7 | 0.0 |
| Osc1-1 | 1 | 57.0 | 0.3 | 0.5 | 0.1 | 42.1 | 0.0 |
| Osc1-2 | 2 | 36.8 | 0.0 | 0.0 | 0.0 | 63.2 | 0.0 |
| Murba1 | 1 | 43.9 | 0.8 | 31.1 | 14.8 | 9.2 | 0.3 |
| Bbac1 | 1 | 46.3 | 8.9 | 4.3 | 4.9 | 35.7 | 0.0 |
| Prev1 | 1 | 41.3 | 29.8 | 14.9 | 7.3 | 6.7 | 0.0 |
| FutA_mut2 | 1 | 42.0 | 0.0 | 30.8 | 3.0 | 24.3 | 0.0 |
| CafC | 1 | 29.5 | 33.7 | 0.0 | 0.7 | 36.0 | 0.0 |
| FucT109 | 1 | 23.9 | 18.0 | 26.6 | 28.7 | 2.0 | 0.8 |
| Csec1 | 1 | 29.1 | 19.2 | 12.8 | 37.7 | 0.9 | 0.2 |
| FutA | 1 | 25.0 | 64.6 | 0.0 | 9.7 | 0.6 | 0.0 |
| FutB | 1 | 0.0 | 0.5 | 6.2 | 78.9 | 1.3 | 13.2 |

HMOs which constitute less than 3% such as less than 2% or such as less than 1% of the total amount of HMOs are considered not to be present in significant amounts. From the data presented in table 7 it can be seen that the six enzymes which are novel in terms of production of fucosylated HMOs, Osc1, Murba1, Csec1, Bbac1, Bacfin1 and Prev1, and the enzymes known to produce less complex fucosylated HMOs, Bgall1, CafC and FutA_mut2, can transfer a fucosyl unit onto the Glu and GlcNAc moieties of LNnT in an α-1,3 linkage at the Glu moiety and the GlcNAc moiety to form LNDFH-III at a level above 25% of the total HMO. The enzyme FucT109, which is known from the prior art to produce LNFP-III and LNFP-VI, surprisingly also shows to be capable of producing 24% LNDFH-III of the total HMO besides production of 18% LNFP-VI, 27% LNFP-III and 29% LNnT, respectively. In fact, FucT109 appears to be the only enzyme that produces all four HMO species in levels above 15%, hence if it is desired to produce a mixture of HMOs where LNDFH-III, LNFP-III, LNFP-VI and LNnT all are presented at levels above 15% this would indeed be an interesting enzyme.

Interestingly of the prior art enzymes which have been reported to produce LNDFH-III, FutA was the only one capable of producing LNDFH-III, resulting in 25% LNDFH-III of the total amount of HMO produced. Furthermore, FutA appeared to produce significantly more LNFP-VI resulting in an LNDFH-III:LNFP-VI ratio of 0.4, contrary to all the novel LNDFH-III producers which all had an LNDFH-III:LNFP-VI ratio above 1.3. FutA also did not produce any LNPF-III at all indicating that the FutA can only fucosylated the GlcNAc moiety of the LNnT backbone with a fucosyl moiety being present on the Glc moiety, e.i., FutA can only fucosylate the GlcNAc moiety of LNFP-VI but not of LNnT.

FutB did not produce any LNDFH-III in the current assay, and in general appeared to be a very poor fucosyltransferase under the present conditions since the main products produced by the FutB strain were the non-fucosylated LNnT and pLNnH HMOs.

From table 7 is can be seen that in cells expressing Osc1 the production of LNFP-III and LNFP-VI is negligible, indicating that Osc1 is very efficient in the fucosylation of both the Glc and GlcNAc moieties on LNnT. Osc1 does however also have quite a high activity on lactose, resulting in 42% 3FL. However, since 3FL is significantly easier to separate from LNDFH-III than LNFP-III and LNFP-VI this strain would be advantageous if it is desired to produce pure LNDFH-III. Furthermore, it can be seen that increasing the copy number of Osc1 to two genetic copies results in a slight increase in the production of 3FL, while abolishing production of LNFP-III and LNFP-VI completely from the strain indicating that Osc1 may prefer lactose over LNnT as substrate.

Bgall1 is the best LNDFH-III producer with 67% LNDFH-III of the total HMO produced, however compared to Osc1 the LNFP-III levels are a bit higher here for Bgall1. As with Osc1, increasing the copy number of Bgall1 to two genomic copies slightly decreased the relative amount of LNDFH-III produced from 67% to 52% of the total HMO content, combined with a significant reduction in the relative LNFP-III amount produced, from 8% to 3% and an increase in the amount of 3FL produced from 24% to 45% respectively. So also here it appears that Bgall1 prefer lactose over LNnT as substrate.

Accordingly, the copy number variation of Bgall1 and Osc1 may be used to tailor specific HMOs mixtures, in this case a mixture comprising of LNDFH-III and 3FL in different ratios, depending on the need for the specific product.

As mentioned above low amounts or absence of alternative fucosylated species, of same or similar size, in the produced mixture is highly advantageous and preferred if it is desired to purify the produced LNDFH-III, thus Bgall1 and Osc1 are especially suited for this purpose.

The enzymes Murba1 Bacfin1 and FutA_mut2 were found to produce mixtures of LNDFH-III, LNFP-III, LNnT and 3FL. These enzymes may be particular useful if a mixture with an LNDFH-III:LNFP-III ratio of 1:1 to 1:3 is desired. Prev1, Bbac1, Csec1 and FucT109 were found to produce a highly complex mixture of HMOs comprising LNDFH-III, LNFP-III, LNFP-VI, LNT and 3FL, thus producing in total 5 different HMOs from a single cell, where Csec1 and FucT109 both produced quite low amounts of 3FL (between 1 and 2%, respectively). The enzyme CafC was found to produce a mixture of HMOs comprising essentially of fucosylated HMOs, namely, LNDFH-III, LNFP-VI and 3FL, with less than 1% LNnT produced 3FL.

### Example 2—Fermentation Using Bgall1 or Osc1 α-1,3/4-Fucosyltransferase Strain for LNDFH-III Production

To confirm the HMO profile observed in the deep well assays, and especially the content of LNDFH-III in the total HMO produced, the Osc1-1 and Bgall1-1 strains of example 1, containing a single genomic copy of Osc1 or Bgall1, the strains were fermented as described in the “Method” section above. The results are shown in table 8.

TABLE 8
Content of individual HMO's as % of
total HMO content produced by the strain

| | | LNT- | LNFP- | LNFP- | LNDFH- | |
|---|---|---|---|---|---|---|
| Strain ref. | 3FL | II | III | VI | III | LNnT |
| Bgall1-1 | 18.3 | 0.4 | 6.9 | 0.0 | 74.2 | 0.2 |
| Osc1-1 | 50.8 | 0.6 | 0.7 | 0.0 | 47.8 | 0.1 |

From the data presented table 8, it can be seen that the fraction of LNDFH-III for the Osc1-1 and Bgall1 strains were similar in the fermentation, as the results presented in example 1, with a tendency of Bgall1 forming a bit more LNDFH-III and less 3FL, whereas Osc1 forms a bit more 3FL than LNDFH-III. Furthermore both strains show the ability and suitability of Bgall1 and Osc1 for the production of LNDFH-III with only low production of alternative complex fucosylated HMOs (LNFP-III and LNFP-VI) in fermentation, in particular Osc1 has very low of both LNFP-III and LNFP-VI as well as LNnT, allowing for the opportunity to obtain very pure LNDFH-III when the broth from this strain is subjected to purification.

### Example 3—Regeneration and Viability of Lyophilized Lactobacillus Species

Probiotics may be consumed as live bacteria or as a dried (e.g. lyophilized) product. Independent of the drying method, rehydration involves an important step in the recovery of dehydrated bacteria; an inadequate rehydration/regeneration step may lead to poor cell viability and a low final survival rate. Rehydration is therefore a highly critical step in the revitalization of a lyophilized culture. For both live and rehydrated bacteria, the survival of the bacteria under acidic conditions is critical since they need to pass through the acidic environment of the stomach and may also be faced with storage (shelf-life) in acidic food products.

In the present example it was tested whether the mixture of HMOs similar to the ones produced by the strains described in example 1 and 2 can provide a benefit in the rehydration (regeneration) and viability of the probiotics. The test was performed under acidic conditions to resemble the conditions bacteria have to survive when passing through the stomach or when dosed in an acidic beverage.

Three different Lactobacillus strains were tested
Lactobacillus rhamnosus Probio-Tec® LGG®-DSM 33156
Lactobacillus rhamnosus LBrGG-ATCC53103
Lactobacillus paracasei L26-CBS116412

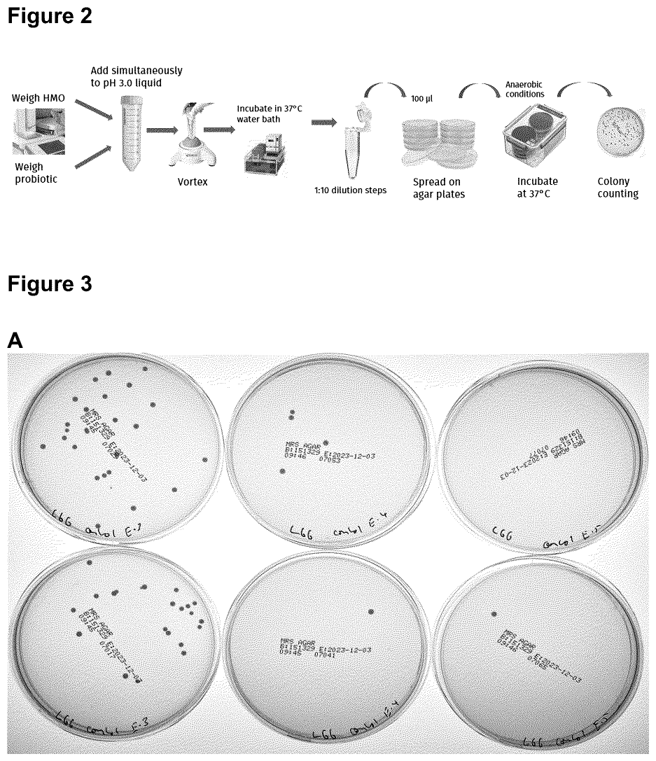

The lyophilized probiotic was added to the tube (0.4 mg/ml), alone (control) or in combination with HMO mixtures (5% w/v) as indicated in table 9. The composition was dissolved in sterile phosphate-buffered saline (PBS, pH=3), warmed to 37° C. and vigorously mixed for about 30 sec until no visible clumps remained. The tubes were incubated at 37° C. for 3 h. The samples were further diluted and 100 μl were spread in duplicates onto MRS agar plates which were incubated at 37° C. in anaerobic chambers. For the experimental setup, see FIG. 2.

TABLE 9
HMO compositions tested in the present example

| HMO | C | Mix1 | Mix2 | Mix3 | Mix4 |
|---|---|---|---|---|---|
| LNDFH-III | 0 | 50 | 70 | 60 | 45 |
| LNFP-III | 0 | 0 | 10 | 20 | 30 |
| 3FL | 0 | 50 | 20 | 10 | 10 |
| LNnT | 0 | 0 | 0 | 10 | 15 |

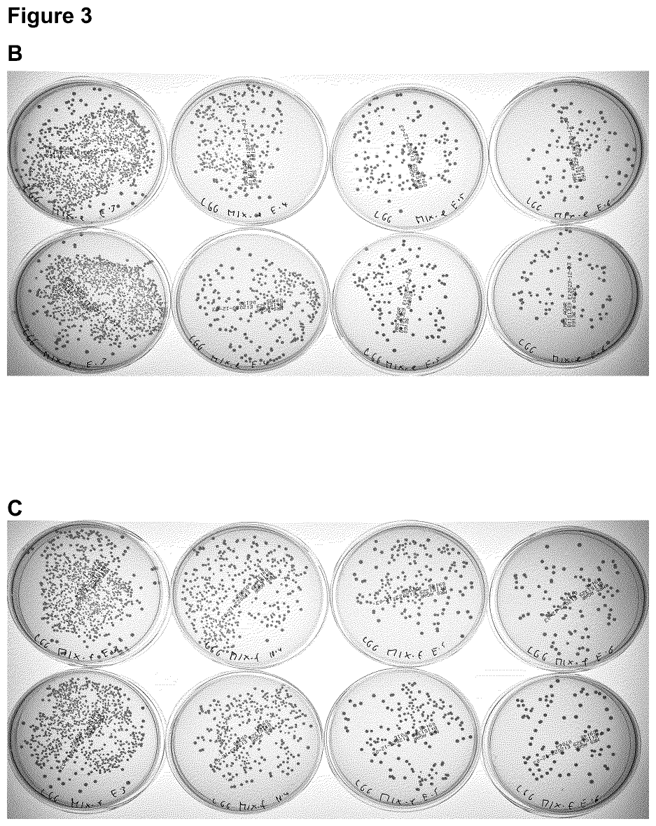

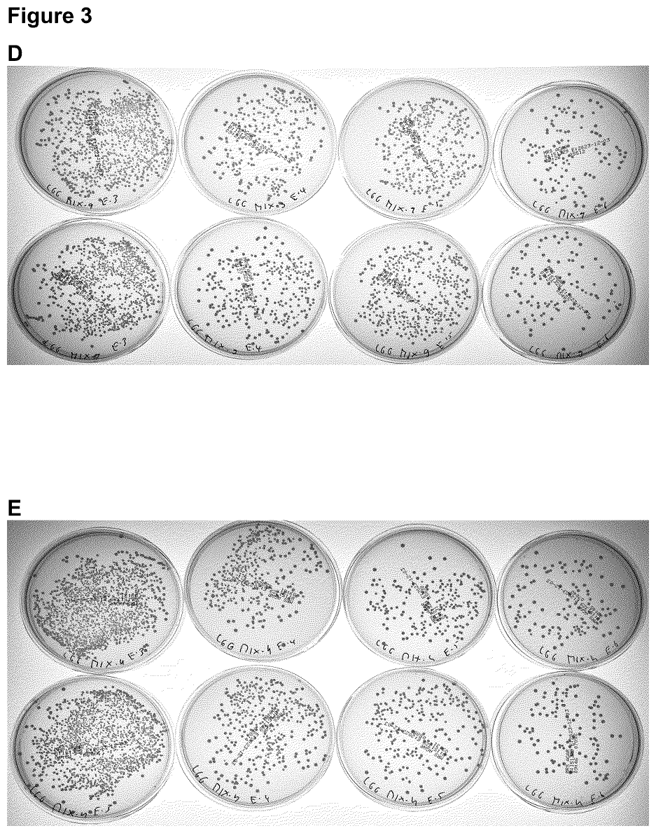

The CFU/ml was calculated based on colonies counted 48 hours after incubation (average of two plates). FIG. 3 shows pictures of the plates with the colonies of Lactobacillus rhamnosus DSM 33156 after 48 hours incubation. The plates for the other two strains are not shown, but they looked similar. The results for all three strains are summarized in table 10.

TABLE 10
Average CFU/ml for the indicated strains after 3 h acid
treatment followed by 48 h subsequent incubation at 37 °C

| | CFU/ml Average | | | | |
|---|---|---|---|---|---|
| Probiotic strain | control | Mix1 | Mix2 | Mix3 | Mix4 |
| L. rhamnosus LGG ®-DSM 33156 | 2.40 × 10⁵ | 8.05 × 10⁸ | 8.45 × 10⁸ | 1.13 × 10⁹ | 1.11 × 10⁹ |
| L. rhamnosus LBrGG | 0.0 | 9.30 × 10⁶ | 2.55 × 10⁶ | 1.62 × 10⁶ | 4.80 × 10⁶ |
| L. paracasei L26 | 2.30 × 10⁴ | 4.95 × 10⁴ | 6.55 × 10⁴ | 1.36 × 10⁵ | 7.30 × 10⁴ |

All the lyophilized Lactobacillus strains dissolved with the HMO mixtures described herein showed an enhanced regeneration and survivability compared to control without the HMO mixtures. These data clearly show that the regeneration and viability of Lactobacillus rhamnosus and L. paracasei strains after exposure to low pH conditions, such as in the stomach or in an acidic beverage, can be improved in the presence of any of the HMO mixtures.

For L. rhamnosus LGG®-DSM 33156 it can also be seen that substituting some of the 3FL in mix 1 with LNnT and LNFP-III increases the regeneration and viability even more than what was observed for mixture 1 and 2.

For L. rhamnosus LBrGG it appears that it is a benefit to have a mixture of just LNDFH-III and 3FL (mix1) although mix 4 indicate that LNFP-III can substitute some of the 3FL. It is also worth noting that L. rhamnosus LBrGG in the absence of an HMO mixture is incapable of surviving acidic treatment.

For L. paracasei L26 mixture 3, which contained all 4 HMOs and 80% of the mixture was LNDFH-III and LNFP-III supplemented with 10% of each of LNnT and 3FL, seemed to have the largest benefit on regeneration and viability.

To our knowledge it has not previously been shown that the tested mixtures provide a benefit of improving the regeneration and survivability of lactobacillus strains in an acidic environment.

### Example 4-Regeneration and Viability of Lyophilized Bifidobacterium Species

As in example 3 above the HMO mixtures in table 9 were also tested for their ability to provides a benefit of improving the regeneration and survivability of Bifidobacterium adolescentis (DSM 34065) in an acidic environment.

Lyophilized probiotic, Bifidobacterium adolescentis (DSM 34065) (0.4 mg/ml), alone or in combination with HMOs mixtures (5% w/v) as indicated in table 9, were dissolved into sterile pH 3.0 water, warmed to 37° C., and vigorously mixed for about 30 seconds until no visible clumps remained. The tubes were incubated at 37° C. for 30 minutes. Afterwards 100 μl were spread in duplicates onto MRS cysteine agar plates which were incubated for 72 h at 37° C. in anaerobic chambers. The regeneration and viability of the probiotics were determined by counting the colonies on the plates after 72 h of incubation.

The CFU/ml was calculated based on colonies counted on undiluted plates 72 hours after incubation (average of two plates). The results are shown in table 11.

TABLE 11
Average CFU/ml of Bifidobacterium adolescentis (DSM 34065) after
30 min acid treatment and 72 h subsequent incubation at 37 °C

CFU/ml Average

| Probiotic strain | control | Mix1 | Mix2 | Mix3 | Mix4 |
|---|---|---|---|---|---|
| Bifidobacterium | 85 | 1250 | 4065 | 3250 | 1280 |
| adolescentis | | | | | |
| (DSM 34065) | | | | | |

As can be seen from table 11, the mixtures are capable making some Bifidobacterium adolescentis (DSM 34065) strain survive acid treatment compared to the control without HMOs. Mixture 2, with the largest amount of LNDFH-III in combination with the two other fucosylated HMOs 3FL and LNFP-III appeared to have the largest effect on regeneration and survivability of Bifidobacterium adolescentis (DSM 34065).

1. A genetically engineered cell capable of producing LNDFH-III, comprising a recombinant nucleic acid sequence encoding an α-1,3-fucosyltransferase selected from the group consisting of,
a. Osc1 comprising the amino acid sequence of SEQ ID NO: 1, or a functional homologue thereof with an amino acid sequence that is at least 9089% identical to SEQ ID NO: 1,
b. Bgall1 comprising the amino acid sequence of SEQ ID NO: 2, or a functional homologue thereof with an amino acid sequence that is at least 9080% identical to SEQ ID NO: 2,
c. Bbac1 comprising the amino acid sequence of SEQ ID NO: 3, or a functional homologue thereof with an amino acid sequence that is at least 89.99% identical to SEQ ID NO: 3,
d. Murba1 comprising the amino acid sequence of SEQ ID NO: 4, or a functional homologue thereof with an amino acid sequence that is at least 90% identical to SEQ ID NO: 4,
e. Bacfin1 comprising the amino acid sequence of SEQ ID NO: 5, or a functional homologue thereof with an amino acid sequence that is at least 90% identical to SEQ ID NO: 5,
f. Prev1 comprising the amino acid sequence of SEQ ID NO: 6, or a functional homologue thereof with an amino acid sequence that is at least 90% identical to SEQ ID NO: 6,
g. Csec1 comprising the amino acid sequence of SEQ ID NO: 7, or a functional homologue thereof with an amino acid sequence that is at least 90% identical to SEQ ID NO: 7,
h. CafC comprising the amino acid sequence of SEQ ID NO: 8, or a functional homologue thereof with an amino acid sequence that is at least 90% identical to SEQ ID NO: 8 and
i. FutA variants comprising substitutions at a position corresponding to position 128 and 129 of SEQ ID NO: 11, wherein the variants have at least 90% sequence identity to SEQ ID NO: 11, and
wherein the cell further comprises one or more recombinant nucleic acid sequence(s) encoding a β-1,4-galactosyltransferase and optionally a β-1,3-N-acetylglucosaminyltransferase.

2. The genetically engineered cell according to claim 1, wherein at least 25% of the molar content of the total HMOs produced by the cell is LNDFH-III.

3. The genetically engineered cell according to claim 1, wherein the cell further produces one or more HMOs selected from the group consisting of 3FL, LNnT, LNFP-III and LNFP-VI.

4. The genetically engineered cell according to claim 1, wherein the cell further comprises one or more recombinant nucleic acid sequence(s) encoding a β-1,4-galactosyltransferase.

5. The genetically engineered cell according to claim 1, wherein the cell further comprises a substrate importer selected from a pre lactose importer, a lacto-N-triose-II (LNT-II) importer or a LNnT importer.

6. The genetically engineered cell according to claim 1, wherein the engineered cell is selected from the group consisting of Escherichia coli, Bacillus subtilis, Lactobacillus lactis, Corynebacterium glutamicum, Yarrowia lipolytica, Pichia pastoris, and Saccharomyces cerevisiae.

7. A method for producing one or more fucosylated HMOs comprising the following steps
a. providing and culturing a genetically engineered cell comprising a recombinant nucleic acid sequence encoding an α-1,3-fucosyltransferase selected from the group consisting of,
i. Osc1 comprising the amino acid sequence of SEQ ID NO: 1, or a functional homologue thereof with an amino acid sequence that is at least 90% identical to SEQ ID NO: 1,
ii. Bgall1 comprising the amino acid sequence of SEQ ID NO: 2, or a functional homologue thereof with an amino acid sequence that is at least 90% identical to SEQ ID NO: 2,
iii. Bbac1 comprising the amino acid sequence of SEQ ID NO: 3, or a functional homologue thereof with an amino acid sequence that is at least 90% identical to SEQ ID NO: 3,
iv. Murba1 comprising the amino acid sequence of SEQ ID NO: 4, or a functional homologue thereof with an amino acid sequence that is at least 90% identical to SEQ ID NO: 4,
v. Bacfin1 comprising the amino acid sequence of SEQ ID NO: 5, or a functional homologue thereof with an amino acid sequence that is at least 20% identical to SEQ ID NO: 5,
vi. Prev1 comprising the amino acid sequence of SEQ ID NO: 6, or a functional homologue thereof with an amino acid sequence that is at least 90% identical to SEQ ID NO: 6,
vii. Csec1 comprising the amino acid sequence of SEQ ID NO: 7, or a functional homologue thereof with an amino acid sequence that is at least 90% identical to SEQ ID NO: 7,
viii. CafC comprising the amino acid sequence of SEQ ID NO: 8, or a functional homologue thereof with an amino acid sequence that is at least 90% identical to SEQ ID NO: 8,
ix. FucT109 comprising the amino acid sequence of SEQ ID NO: 10, or a functional homologue thereof with an amino acid sequence that is at least 90% identical to SEQ ID NO: 10, and
x. FutA variants comprising substitutions at a position corresponding to position 128 and 129 of SEQ ID NO: 11, wherein the variants have at least 90% sequence identity to SEQ ID NO: 11, and
b. culturing the cell according to (a) in a suitable cell culture medium to produce the one or more fucosylated HMOs, wherein at least one of the fucosylated HMOs is LNDFH-III, and
c. optionally, purifying said one or more fucosylated HMOs.

8. The method according to claim 7, wherein at least 25% of the molar content of the total HMOs produced by the method is LNDFH-III.

9. The method according to claim 7, wherein at least one additional fucosylated HMOs is produced and is selected from the group consisting of 3FL, LNFP-III and LNFP-VI.

10. The method according to claim 7, wherein the genetically engineered cell is cultivated in the presence of an acceptor substrate selected from the group consisting of lactose, LNT-II and LNnT.

11. (canceled)

12. A mixture of HMOs produced with a method according to claim 7, consisting essentially of
a. LNDFH-III and 3FL, or
b. LNDFH-III, LNFP-III and 3FL, or
c. LNDFH-III, LNFP-VI and 3FL, or
d. LNDFH-III, LNFP-III, LNFP-VI and LNnT, or
e. LNDFH-III, LNFP-III, 3FL and LNnT, or
f. LNDFH-III, LNFP-III, LNFP-VI, 3FL and LNnT.

13. A composition of HMOs consisting essentially of 20-70 molar % of LNDFH-III, 0-35 molar % LNFP-III, 0-35 molar % LNFP-VI, 0-65 molar % 3FL, 0-40% LNnT, and below 1% pLNnH, in total adding up to 100% molar content.

14. The composition according to claim 13, wherein the composition comprises a mixture of HMOs selected from the group consisting of:
a. a mixture consisting essentially of 35-60 molar % of LNDFH-III and 40-65 molar % 3FL, in total adding up to 100% molar content,
b. a mixture consisting essentially of 50 molar % of LNDFH-III and 50 molar % 3FL,
c. a mixture consisting essentially of 40-50 molar % LNDFH-III, 25-35 molar % LNFP-VI, 10-20 molar % LNnT and 5-15 molar % 3FL, in total adding up to 100% molar content,
d. a mixture consisting essentially of 25-35 molar % LNDFH-III, 15-25 molar % LNFP-VI, 8-18 molar % LNFP-III and 33-43 molar % LNnT, in total adding up to 100% molar content,
e. a mixture consisting essentially of 40-50 molar % LNDFH-III, 5-15 molar % LNFP-VI, 3-8 molar % LNFP-III and 2-12 molar % LNnT and 30-40 molar % 3FL, in total adding up to 100% molar content,
f. a mixture consisting essentially of 55-65 molar % LNDFH-III, 17-27 molar % LNFP-III and 6-16 molar % LNnT and 1-11 molar % 3FL, in total adding up to 100% molar content,
g. a mixture consisting essentially of 60 molar % LNDFH-III, 20 molar % LNFP-III and 10 molar % LNnT and 10 molar % 3FL,
h. a mixture consisting essentially of 36-46 molar % LNDFH-III, 25-35 molar % LNFP-VI, 10-20 molar % LNFP-III and 2-12 molar % LNnT and 2-12 molar % 3FL, in total adding up to 100% molar content,
i. a mixture consisting essentially of 50-70 molar % LNDFH-III, 1-13 molar % LNFP-III and 20-50% 3FL, in total adding up to 100% molar content,
j. a mixture consisting essentially of 70 molar % LNDFH-III, 10 molar % LNFP-III and 20-50% 3FL,
k. a mixture of consisting essentially of 36-46 molar % LNDFH-III, 25-35 molar % LNFP-VI, 10-20 molar % LNFP-III and 2-12 molar % LNnT and 2-12 molar % 3FL, in total adding up to 100% molar content,
l. a mixture consisting essentially of 45 molar % LNDFH-III, 30 molar % LNFP-III, 10 molar % 3FL, and 15% LNnT,
m. a mixture consisting essentially of 25-35 molar % LNDFH-III, 29-39 molar % LNFP-VI and 31-41% 3FL, in total adding up to 100% molar content,
n. a mixture consisting essentially of 37-47 molar % LNDFH-III, 26-36 molar % LNFP-III, 19-29 molar % 3FL, and less than 5% LNnT, in total adding up to 100% molar content, and
o. a mixture consisting essentially of 19-29 molar % LNDFH-III, 13-23 molar % LNFP-VI, 22-32 molar % LNFP-III and 24-34 molar % LNnT and less than 5 molar % 3FL, in total adding up to 100% molar content.

15. A composition comprising a probiotic strain selected from one or more Bifidobacterium and/or Lactobacillus species and a mixture of HMOs according to claim 12.

16. (canceled)

17. The genetically engineered cell according to claim 1, wherein the recombinant nucleic acid sequence encoding an α-1,3-fucosyltransferase is Osc1 comprising the amino acid sequence of SEQ ID NO: 1, or a functional homologue thereof with an amino acid sequence that is at least 90% identical to SEQ ID NO: 1.

18. The genetically engineered cell according to claim 1, wherein the recombinant nucleic acid sequence encoding an α-1,3-fucosyltransferase is Bgall1 comprising the amino acid sequence of SEQ ID NO: 2, or a functional homologue thereof with an amino acid sequence that is at least 90% identical to SEQ ID NO: 2.

19. The genetically engineered cell according to claim 1, wherein the recombinant nucleic acid sequence encoding an α-1,3-fucosyltransferase is Bacfin1 comprising the amino acid sequence of SEQ ID NO: 5, or a functional homologue thereof with an amino acid sequence that is at least 90% identical to SEQ ID NO: 5

19. The genetically engineered cell according to claim 1, wherein the cell further comprises one or more recombinant nucleic acid sequences encoding a β-1,4-galactosyltransferase and a β-1,3-N-acetylglucosaminyltransferase.

20. The genetically engineered cell according to claim 6, wherein the engineered cell is selected from the group consisting of Escherichia coli.
